(19) United States
(12) Patent Application Publication
(10) Pub. No.: US 2016/0050895 A1
(43) Pub. Date: Feb. 25, 2016

(54) TRANSGENIC NON-HUMAN ORGANISMS WITH NON-FUNCTIONAL TSPO GENES

(71) Applicants: AUSTRALIAN NUCLEAR SCIENCE AND TECHNOLOGY ORGANIZATION, Lucas Heights, New South Wales (AU); THE UNIVERSITY OF SYDNEY, Sydney, New South Wales (AU)

(72) Inventors: Richard BANATI, Grays Point (AU); Guo Jun LIU, Penshurst (AU); Ryan MIDDLETON, Leichhardt (AU)

(21) Appl. No.: 14/775,650

(22) PCT Filed: Mar. 13, 2014
(86) PCT No.: PCT/AU2014/000250

§ 371 Date: Sep. 11, 2015

(30) Foreign Application Priority Data
Mar. 13, 2013 (AU) 2013900858
Sep. 25, 2013 (AU) 2013903696
Dec. 24, 2013 (AU) 2013905101

(51) Int. Cl. A01K 67/027; G01N 33/50

(52) U.S. Cl. CPC A01K 67/0276; G01N 33/5088; A01K 2227/105; A01K 2267/0312; A01K 2267/0318

(57) ABSTRACT

The present invention relates to transgenic animal models. Specifically, the present invention relates to transgenic animal models for applications associated with TSPO-related normal physiology, diseases and disorders. The present invention features a transgenic nonhuman animal comprising cells with at least one copy of a non-functional, endogenous TSPO gene. Also disclosed are compounds for investigating or modulating TSPO-related functions.

### CROSS-REFERENCE

This application claims priority from Australian provisional patent application 2013900858 filed 13 Mar. 2013. Australian provisional patent application 2013903696 filed 25 Sep. 2013 and Australian provisional patent application 2013905101 filed 24 Dec. 2013, all of which are herein incorporated by cross-reference in their entirety.

### TECHNICAL FIELD

The present invention relates to transgenic animal models. Specifically, the present invention relates to transgenic animal models for applications associated with TSPO-related normal physiology, diseases and disorders.

### BACKGROUND OF THE INVENTION

Translocator protein (TSPO), previously known as the peripheral benzodiazepine receptor (PBR), is an 18 kDa membrane protein primarily located on the outer mitochondrial membrane and is widely distributed throughout the body. Its expression level varies in different tissues and organs. Healthy adult brain parenchyma in particular has a very low level of TSPO, while the kidney, lung and heart express high levels, and even higher levels of TSPO are present in glandular and steroid-producing tissues. TSPO is relatively well conserved across species, from bacteria to insects to mammals.

The primary function of TSPO appears to be associated with steroid production, but TSPO has also been implicated in protein transport, ion transport, porphrin transport and heme biosynthesis, cellular proliferation and differentiation, regulation of mitochondrial function, cellular respiration, gluconeogenesis and involvement in oxidative processes, and apoptosis. Unbiased large-scale whole-organism screening identifies the TSPO as a protein of central importance, confirms that the function of the TSPO are well preserved across species and that observations made in one species can be translated to another species. This is independent evidence that any organism in which the TSPO is altered is of high utility in the study of many fundamentally important biological functions in health and disease.

Again, according to current concepts, the most important function of TSPO is the regulation of steroid hormone production by helping to translocate cholesterol, the precursor to pregnenolone, across the aqueous mitochondrial intermembrane space, a view emphasised by the renaming of the PBR as TSPO. A large body of literature on the regulatory influence of the TSPO on steroid-mediated homeostasis, including the reported embryonic lethal effects of a TSPO gene knock-out, has established the view that TSPO gene products are essential for life. As indicated above, TSPO's critical role as an endocrine regulator is the prevailing explanation for the observed actions of TSPO binding compounds across an exceptionally broad therapeutic spectrum ranging from the anti-inflammatory treatment of Alzheimer's disease (Barron, A. M. et al. Ligand for translocator protein reverses pathology in a mouse model of Alzheimer's disease. J Neurosci 33, 8891-8897, doi: 10.1523/JNEUROSCI. 1350-13.2013 (2013)) to anxiolysis without direct effects on the central GABA_{A} receptor protein complex (Rupprecht. R. et al. Translocator protein (18 kDa) (TSPO) as a therapeutic target for neurological and psychiatric disorders. Nat Rev Drug Discov 9, 971-988, doi:nrd3295 [pii]10.1038/nrd3295 (2010); Rupprecht, R. et al. Translocator protein (18 kD) as target for anxiolytics without benzodiazepine-like side effects. Science 325, 490-493, doi:10.1126/science. 1175055 (2009)). As such, TSPO has been implicated in numerous pathological or disease conditions.

Further to the above, these include diseases with neuroinflammation, neurodegenerative diseases, brain injury, ischemia-reperfusion injury, epilepsy and cancer, where TSPO is highly up regulated. The use of TSPO as a target for imaging is particularly useful in the brain where TSPO has relatively low expression. On a more basic level, TSPO has become a target for imaging and diagnostic tools and has been proposed as a therapeutic target for various neurological and psychiatric disorders.

Several classes of ligands have been shown to exhibit high affinity binding to peripheral benzodiazepine receptors, the most widely investigated being the benzodiazepine Ro 5-4864 (7-chloro-5-(4-chlorophenyl)-1,3-dihydro-1-methyl-2H-1,4-benzodiazepin-2-one) and the isoquinoline PK-11195 (1-(2-chlorophenyl)-N-methyl-N-(1-r-methylpropyl)-3-isoquinolinecarboxamide). Labelled with ¹¹C, ¹⁸F and ¹²³I, these ligands have been used to map peripheral benzodiazepine receptors in the human heart and brain. Enhanced uptake of [³H]PK-11195 has been reported in a variety of tumour cells including breast, ovarian, prostate, adrenal, brain and colon. Radiolabelling with suitable levels of radioactive iodine may be used firstly to diagnose these tumours (using radiolabels such as ¹²³I or ¹³¹I) and subsequently to treat them with therapeutic doses (for instance using ¹²³I, ¹²⁵I or ¹³¹I).

Compounds which exhibit high affinity to peripheral benzodiazepine receptors such as TSPO have been described as also exhibiting strong binding to the central benzodiazepine receptors (see for example Anzini M. et al., J. Med Chem. 39 4275-4284 (1996) and Trapani G. et al. J. Med. Chem. 40 3109-3118 (1997)). Hence, certain TSPO binding compounds are often not sufficiently selective to be useful for diagnosis or therapy of TSPO-related diseases and conditions.

More recently certain 2-(iodophenyl)-imidazo[1.2-a]pyridines have been described, which bind strongly to peripheral benzodiazepine receptors but do not bind strongly to central benzodiazepine receptors (see U.S. Pat. No. 6,379,649, the entire disclosure of which is incorporated herein by reference). These 2-(iodophenyl)-imidazo[1.2-a]pyridines, when having an electronegative substituent, especially when that substituent is a halogen in the pyridine nucleus, exhibit strong binding to peripheral benzodiazepine receptors and much weaker binding to central benzodiazepine receptors, which makes these compounds useful for the diagnosis and treatment, including radiotherapy, of disorders that are characterized by an abnormal density of peripheral benzodiazepine receptors.

However, and despite the wide applications of TSPO research, there are currently no non-TSPO background cells, tissues or animals for the in situ identification and verification of purported TSPO-binding compounds as bona fide TSPO-binding compounds. Instead, reliability of TSPO research conducted so far is compromised by the possibility that TSPO binding effects seen in the available in vitro competitive ligand binding systems are merely the result of non-specific and non-selective binding bearing no relevance with respect to in vivo TSPO function.

As indicated above, previous efforts to generate a much-needed, TSPO knock-out animal model have failed as they resulted in non-viable embryos, ascribing an “embryo-lethal” phenotype to the TSPO gene knock-out.

As is apparent, an animal model is needed to provide a true negative control for TSPO research. The availability of such a model system would change the way current TSPO research is conducted and would allow retrospective re-evaluation of research findings pertaining to TSPO-related diseases and conditions. Further, an animal in which at least one allele of the TSPO gene is non-functional or absent, would significantly extend the hitherto-limited possibility of studying the regulation of other TSPO-dependent biological pathways. Thus the generation of animals useful for studying TSPO-related diseases and conditions would be a major milestone within the TSPO field.

It is an object of the present invention to overcome or ameliorate at least one of the disadvantages of the prior art, or to provide a useful alternative.

### SUMMARY OF THE INVENTION

Orthologues of TSPO are found widely among bacteria and archaea. Most members of the TSPO protein family contain a specific binding site for the isoquinoline carboxamide PK11195, which has been used to pharmacologically define TSPO and distinguish it from other benzodiazepine binding receptors, such as the GABAA receptor protein complex. An evolutionarily younger C-terminal cholesterol recognition amino acid consensus (CRAC) domain is primarily found in the animal phylum.

TSPO is thought to be involved in mitochondrial energy production and transport of porphyrin intermediates. Its most important function, however, is thought to be the regulation of steroid hormone production by virtue of participating in the translocation of cholesterol, the precursor to pregnenolone, across the aqueous mitochondrial intermembrane space.

Against the prevailing published view and theoretical considerations that changes to, or deletion of, the highly conserved TSPO gene is unlikely to lead to a viable, fertile animal, the inventors have successfully generated a novel, transgenic animal in which the absence of a functional TSPO gene is not lethal.

Specifically the inventors have, surprisingly, shown that global TSPO gene knock-out mice in accordance with the present invention (also referred to as mouse strain C57BL/6-TSPO^{tm1GuWu(GuwiyungWurra)} throughout) are viable with normal cholesterol transport, pregnenolone synthesis, fertility, protoporphyrin IX metabolism and, under healthy conditions, without overt clinical impairment.

However, the absence of TSPO in homozygous TSPO knock-out mice has revealed a role for TSPO in regulating the systemic and/or cellular energy household, in regulating mitochondrial oxidative pathways, mitochondrial ATP production and energy storage in response to high-fat diet. Specifically, the inventors have surprisingly found that increased energy intake in the form of a prolonged, high fat diet leads to a significantly reduced (and less than expected) weight gain in TSPO knock-out animals according to the present invention when compared to wild-type animals. Accordingly, a role for TSPO and TSPO-mediated signalling in the protection against obesity resulting from a high fat diet has been provided.

Furthermore, the TSPO knock-out mice illustrated the surprising finding that global loss of TSPO function has no or only a minimal effect on the activation of microglia after neuronal injury. These results suggested that the current understanding of TSPO function in relation to “neuroinflammation”, too, merited revisiting as they did not adequately distinguish neuro-glial interactions from responses in inflammatory tissues that regularly show high levels of TSPO expression.

Accordingly, the global TSPO gene knock-out animal according to the present invention provides a crucial tool to evaluate the diagnostic and therapeutic selectivity of existing and new chemical compounds with affinity to the TSPO. Reducing the reliance on conjecture, it enables a broad range of fundamental experiments into the controversially discussed pathways of steroid biogenesis, steroid-dependent systemic effects, including behaviour (Rupprecht, R. et al. Translocator protein (18 kD) as target for anxiolytics without benzodiazepine-like side effects. Science 325, 490-493; Miller, W. L. & Auchus, R. J. The molecular biology, biochemistry, and physiology of human steroidogenesis and its disorders. Endocr Rev 32, 81-151; Stocco, D. M. The Role of PBR/TSPO in Steroid Biosynthesis Challenged. Endocrinology 155, 6-9), as well as mitochondrial energy production and the mechanisms of protection against diet-induced obesity (Gut, P. et al. Whole-organism screening for gluconeogenesis identifies activators of fasting metabolism. Nat Chem Biol 9, 97-104; Divakaruni, A. S. & Brand, M. D. The regulation and physiology of mitochondrial proton leak. Physiology 26, 192-205).

Accordingly, in a first aspect the present invention relates to a transgenic non-human animal comprising cells with at least one copy of a non-functional endogenous TSPO gene.

In one embodiment, the cells do not comprise a functional TSPO gene.

In another embodiment, the non-functional endogenous TSPO gene contains at least one mutation selected from the group consisting of a deletion, an insertion, a frame-shift mutation, re-arrangement or a substitution. In further embodiments, the mutation may be constitutive or conditional.

In another embodiment, the mutation comprises a deletion of all or part of exon 1, 2, 3 or 4 within said TSPO gene. In yet another embodiment, the mutation comprises a deletion of all or part of exon 2 or 3 within said TSPO gene. Typically, the mutation is a deletion of exons 2 and 3 within said TSPO gene.

In one embodiment of the present invention, the non-human animal is from a family selected from a group consisting of Drosophila, Hirudinea, Murine or Cyprinidae.

In a further embodiment, the non-human animal is a mouse.

In a second aspect the present invention relates to progeny of any of the non-human animals of the present invention. The progeny may be generated from the breeding of any of the non-human animals of the present invention with any other animal of the same species.

In particular embodiments, the progeny are generated from cross-breeding a mouse of the present invention with a pKZ1 mouse, a Brca2 homozygous mouse, Tg(CAT)(+/+) mouse, an A-T mutated heterozygous/homozygous mouse, a Csbm/m mouse, an insulin-like growth factor 1 heterozygous and homozygous mouse, a P53 heterozygous and homozygous mouse, a radiation sensitive and resistant transgenic mouse, a Schizophrenia DISC1 knock-out mouse, a Schizophrenia neuregulin 1 knock-out mouse, a genetic engineering model tumour mouse, a neuroinflammation model mouse, an Alzheimer's Disease model mouse, a Parkinson's disease model mouse or a mouse with targeted deletion of the type 2 deiodinase gene (D2KO) that is insulin resistant and susceptible to diet induced obesity.

In a third aspect the present invention relates to a method for identifying a compound for use in the treatment of a TSPO-related disease or disorder in a subject, the method comprising administering a candidate compound to any non-human animal of the invention as described herein, and assessing the effects of the candidate compound on the phenotype of the non-human animal.

In a fourth aspect the present invention relates to a method for identifying a compound for use in the treatment of a TSPO-related disease or disorder in a subject, the method comprising administering a candidate compound to any non-human animal of the invention as described herein, and assessing the effects of the candidate compound on the expression levels of TSPO-associated gene products, or any TSPO gene products that may be present in the non-human animal.

In a fifth aspect the present invention relates to a method for screening the binding specificity or selectivity of a candidate compound for use in the treatment of a TSPO-related disease or disorder in a subject, the method comprising administering a candidate compound to any non-human animal of the invention as described herein, and a wild-type non-human animal of the same species, and comparing the binding specificity or selectivity of the candidate compound to TSPO-associated gene products, or any TSPO gene products that may be present in the wild-type non-human animal, with the binding specificity or selectivity of the candidate compound to TSPO-associated gene products, or any TSPO gene products that may be present in the test non-human animal.

In one embodiment the invention relates to the non-human animal of the invention as described herein, when used for identifying a compound for use in the treatment of a TSPO-related disease or disorder in a subject.

In one embodiment, the non-human animal is used for screening the binding specificity or selectivity of a candidate compound for use in the treatment of a TSPO-related disease or disorder in a subject.

In another embodiment, the non-human animal is used for the diagnosis of a TSPO-related disease or disorder in a subject.

In a sixth aspect the present invention relates to a cell, tissue or immortalised cell line derived from an animal of the first aspect or its progeny.

In a seventh aspect the present invention relates to use of the cell, tissue or immortalised cell line of the sixth aspect as a negative control for detecting a TSPO gene product in a biological sample.

In an eighth aspect the present invention relates to use of the cell, tissue or immortalised cell line of the sixth aspect as a negative control for detecting a TSPO gene product in a biological sample from a subject that has, or is suspected to have, a TSPO-related disease or disorder.

In a ninth aspect the present invention relates to use of the cell, tissue or immortalised cell line of the sixth aspect for the diagnosis of a TSPO-related disease or disorder in a subject.

In a tenth aspect the present invention relates to a method for identifying a compound for use in the treatment of a TSPO-related disease or disorder, the method comprising exposing a cell tissue or immortalised cell line of the sixth aspect to a candidate compound, and assessing the effects of the candidate compound on the phenotype of said cell, tissue or immortalised cell line.

In an eleventh aspect the present invention relates to a method for identifying a compound for use in the treatment of a TSPO-related disease or disorder in a subject, the method comprising exposing any cell, tissue or immortalised cell line of the sixth aspect to a candidate compound, and assessing the effects of the candidate compound on the expression levels of TSPO-associated gene products, or any TSPO gene products that may be present in the cell tissue or immortalised cell line.

In a twelfth aspect of the present invention, there is provided a method for screening the binding specificity or selectivity of a candidate compound for use in the treatment of a TSPO-related disease or disorder in a subject, the method comprising exposing a wild-type cell, wild-type tissue or immortalised cell line, and any test cell or immortalised cell line of the invention as herein described, to a candidate compound, and comparing the binding specificity or selectivity of the candidate compound to TSPO-associated gene products, or any TSPO gene products that may be present in the wild-type cell, wild-type tissue or immortalised cell line, with the binding specificity or selectivity of the candidate compound to TSPO-associated gene products, or any TSPO gene products that may be present in the test cell, tissue or immortalised cell line.

In particular embodiments of the present invention, the TSPO-related disease or disorder is selected from the group consisting of cancer, neuroinflammation, Alzheimer's Disease, Parkinson's Disease, Epilepsy, brain injury, Ischemia-reperfusion injury, behaviour or neurological or psychiatric disorders including acute and chronic stress, anxiety disorders, mode disorders, and Schizophrenia, peripheral neuropathy, Multiple Sclerosis, neuropathic pain, obesity, diabetes and cachexia.

In a thirteenth aspect the present invention relates to a compound when identified by any one of the methods of the third, fourth, tenth or eleventh aspects.

In a fourteenth aspect the present invention relates to use of a compound of general formula (I)

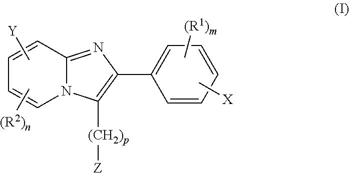

to inhibit TSPO function, wherein

X is absent, iodine or an isotope thereof:

Y is selected from F, Cl, Br, I, OH, SH, NH₂, CN and COOH;

Z is selected from N(R³)C(O)R⁴ and C(O)NR³R⁴;

R¹ and R² are independently selected from (C₁-C₆)alkyl, (C₁-C₆)alkoxy, (C₂-C₆)alkenyl, (C₂-C₆)alkynyl, (C₃-C₆)cycloalkyl, (C₆-C₁₂)aryl, (C₆-C₁₂)aryloxy, (C₆-C₁₂)aryl(C₁-C₆)alkyl, heteroaryl, heteroaryl(C₁-C₆)alkyl, heterocyclic, (C₂-C₆)alkanoyl and (C₂-C₇)acyl, each of which may be unsubstituted or substituted with from 1 to 3 substituents selected from the group consisting of halogen or an isotope thereof, OH, (C₁-C₄)alkoxy, SH, NH₂, (C₁-C₄)alkylamino, di((C₁-C₄)alkyl)amino, carboxy, (C₁-C₄)alkoxycarbonyl, (C₂-C₄)alkanoyl, oxo, amido, CN, CNS, SCN, CNO, OCN and NHOH;

R³ and R⁴ are each independently hydrogen or a group selected from (C₁-C₄)alkyl, (C₂-C₄)alkenyl, (C₂-C₄)alkynyl, (C₃-C₆)cycloalkyl, (C₆-C₁₂)aryl, (C₆-C₁₂)aryl(C₁-C₄)alkyl, heteroaryl, heteroaryl(C₁-C₄)alkyl, heterocyclic, (C₁-C₄)alkoxycarbonyl and (C₂-C₅)acyl, each of which may be unsubstituted or substituted with from 1 to 3 substituents selected from the group consisting of halogen, OH, (C₁-C₄)alkoxy, SH, NH₂, (C₁-C₄)alkylamino, di((C₁-C₄)alkyl)amino, carboxy, (C₁-C₄)alkoxycarbonyl, (C₁-C₄)alkanoyl, oxo, amido, CN, CNS, SCN, CNO, OCN and NHOH,

or R³ and R⁴ together are (C₂-C₇)alkylidene which may be optionally substituted with from 1 to 3 substituents selected from the group consisting of halogen, OH, (C₁-C₄)alkoxy. SH, NH₂, (C₁-C₄)alkylamino, di((C₁-C₄)alkyl)amino, carboxy, (C₁-C₄)alkoxycarbonyl, (C₁-C₄)alkanoyl, oxo, amido, CN, CNS, SCN, CNO, OCN and NHOH;

m and n are independently 0, 1 or 2; and

p is 1:

wherein at each occurrence
(i) alkenyl has the meaning of ethylenically mono- or di-unsaturated alkyl or ethylenically mono- or di-unsaturated cycloalkyl;
(ii) cycloalkyl has the meaning of cyclic alkyl, or alkyl substituted cyclic alkyl;
(iii) heteroaryl has the meaning of single, polynuclear, conjugated and fused residues of aromatic heterocyclic ring systems.

In a fifteenth aspect the present invention relates to use of a compound of general formula (I)

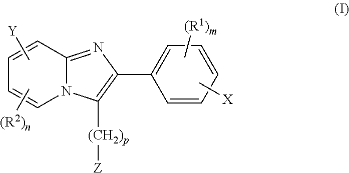

to alter the systemic and/or cellular energy household, wherein

X, Y, Z, R¹, R², R³, R⁴, in, n and p are as defined for the compound of the fourteenth aspect; and

wherein at each occurrence
(i) alkenyl has the meaning of ethylenically mono- or di-unsaturated alkyl or ethylenically mono- or di-unsaturated cycloalkyl;
(ii) cycloalkyl has the meaning of cyclic alkyl, or alkyl substituted cyclic alkyl;
(iii) heteroaryl has the meaning of single, polynuclear, conjugated and fused residues of aromatic heterocyclic ring systems.

In a sixteenth aspect the present invention relates to use of a compound of general formula (I)

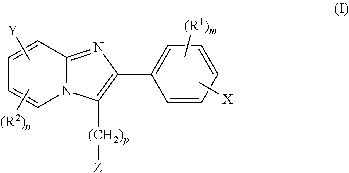

to alter a mitochondrial oxidative pathway, wherein

X, Y, Z, R¹, R², R³, R⁴, m, n and p are as defined for the compound of the fourteenth aspect; and

wherein at each occurrence
(i) alkenyl has the meaning of ethylenically mono- or di-unsaturated alkyl or ethylenically mono- or di-unsaturated cycloalkyl;
(ii) cycloalkyl has the meaning of cyclic alkyl, or alkyl substituted cyclic alkyl;
(iii) heteroaryl has the meaning of single, polynuclear, conjugated and fused residues of aromatic heterocyclic ring systems.

In a seventeenth aspect the present invention relates to use of a compound of general formula (I)

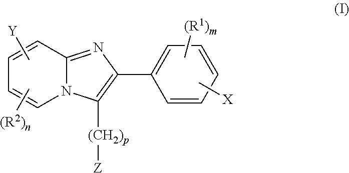

to regulate mitochondrial ATP production, wherein

X, Y, Z, R¹, R², R³, R⁴, m, n and p are as defined for the compound of the fourteenth aspect; and

wherein at each occurrence
(i) alkenyl has the meaning of ethylenically mono- or di-unsaturated alkyl or ethylenically mono- or di-unsaturated cycloalkyl;
(ii) cycloalkyl has the meaning of cyclic alkyl, or alkyl substituted cyclic alkyl;
(iii) heteroaryl has the meaning of single, polynuclear, conjugated and fused residues of aromatic heterocyclic ring systems.

In an eighteenth aspect the present invention relates to use of a compound of general formula (I)

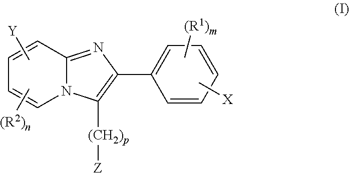

to regulate TSPO-mediated signalling, wherein

X, Y, Z, R¹, R², R³, R⁴, m, n and p are as defined for the compound of the fourteenths aspect; and

wherein at each occurrence
(i) alkenyl has the meaning of ethylenically mono- or di-unsaturated alkyl or ethylenically mono- or di-unsaturated cycloalkyl;
(ii) cycloalkyl has the meaning of cyclic alkyl, or alkyl substituted cyclic alkyl;
(iii) heteroaryl has the meaning of single, polynuclear, conjugated and fused residues of aromatic heterocyclic ring systems.

In a nineteenth aspect the present invention relates to use of a compound of general formula (I)

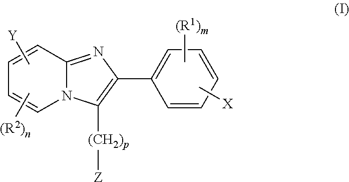

to regulate TSPO-mediated energy storage, wherein

X, Y, Z, R¹, R², R³, R⁴, m, n and p are as defined for the compound of the fourteenth aspect; and

wherein at each occurrence
(i) alkenyl has the meaning of ethylenically mono- or di-unsaturated alkyl or ethylenically mono- or di-unsaturated cycloalkyl;
(ii) cycloalkyl has the meaning of cyclic alkyl, or alkyl substituted cyclic alkyl;
(iii) heteroaryl has the meaning of single, polynuclear, conjugated and fused residues of aromatic heterocyclic ring systems.

In one embodiment of the invention, use of any one of the fourteenth to nineteenth aspects, provides protection against obesity. Typically, use of any one of the fourteenth to nineteenth aspects, provides protection against high fat diet-induced weight gain.

In a twentieth aspect the present invention relates to use of a compound of general formula (I)

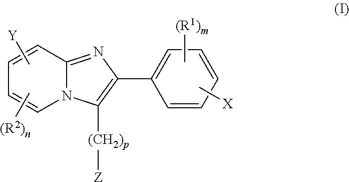

to investigate inflammatory responses associated with neuronal injury, wherein

X, Y, Z, R¹, R², R³, R⁴, m, n and p are as defined for the compound of the fourteenth aspect; and

wherein at each occurrence
(i) alkenyl has the meaning of ethylenically mono- or di-unsaturated alkyl or ethylenically mono- or di-unsaturated cycloalkyl;
(ii) cycloalkyl has the meaning of cyclic alkyl, or alkyl substituted cyclic alkyl;
(iii) heteroaryl has the meaning of single, polynuclear, conjugated and fused residues of aromatic heterocyclic ring systems.

In some embodiments of the present invention, n is 0.

In alternative embodiments Y is selected from F, Cl, Br, I, CN and OH; Z is selected from N(R³)C(O)R⁴ and C(O)NR³R⁴; R¹ and R² are independently selected from (C₁-C₃)alkyl, (C₁-C₃)alkoxy, (C₂-C₃)alkenyl, (C₅-C₆)cycloalkyl, phenyl, naphthyl, phenoxy, naphthyloxy, benzyl, pyridyl, furanyl, thienyl, piperidinyl, morpholinyl, tetrahydrofuranyl, dioxanyl, (C₂-C₄)alkanoyl and (C₂-C₄)acyl, each of which may be unsubstituted or substituted with from a substituent selected from the group consisting of halogen, OH, (C₂-C₄)alkoxy, NH₂, (C₁-C₃)alkylamino, di((C₁-C₃)alkyl)amino, carboxy, (C₁-C₃)alkoxycarbonyl, (C₂-C₄)alkanoyl, oxo and amido; R³ and R⁴ are each independently hydrogen or a group selected from (C₁-C₃)alkyl, (C₂-C₃)alkenyl, (C₅-C₆)cycloalkyl, phenyl, naphthyl, benzyl and (C₂-C₄)acyl, each of which may be unsubstituted or substituted with a substituent selected from the group consisting of halogen, OH, (C₁-C₃)alkoxy, NH₂, (C—C₃)alkylamino, di((C₁-C₃)alkyl)amino, carboxy, (C₁-C₃)alkoxycarbonyl, (C₂-C₄)alkanoyl, oxo and amido, or R³ and R⁴ together are (C₂-C₃)alkylidene which may be optionally substituted with from a substituent selected from the group consisting of halogen, OH, (C₁-C₃)alkoxy, NH₂, (C₁-C₃)alkylamino, di((C₁-C₃)alkyl)amino, carboxy, (C₁-C₃)alkoxycarbonyl, (C₂-C₄)alkanoyl, oxo and amido; and m and n are independently 0 or 1.

In some embodiments the compound of formula (I) is a 2-(4′-iodophenyl)-imidazol[1,2-a]pyridine-3-acetamide derivative of formula (IA)

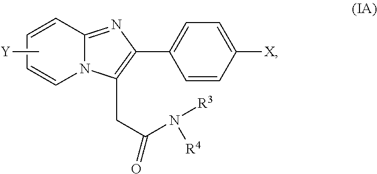

wherein: X is iodine or an isotope thereof, Y is halogen; and R³ and R⁴ are independently selected from hydrogen, (C₁-C₄)alkyl and (C₂-C₄)alkenyl, or R³ and R⁴ taken together are (C₂-C₃)alkylidine.

Typically, X is ¹²⁵I; Y is Cl; and R³ and R⁴ are CH₂CH₃, i.e. the compound is [¹²⁵I]CLINDE.

In alternative embodiments of the present invention, compound of formula (I) is a derivative of formula (IB)

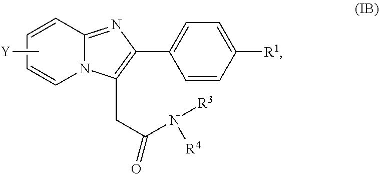

wherein: Y is halogen; R¹ is independently selected from (C₁-C₆)alkyl, (C₁-C₆)alkoxy, (C₂-C₆)alkenyl, (C₂-C₆)alkynyl, (C₃-C₆)cycloalkyl, (C₆-C₁₂)aryl, (C₆-C₁₂)aryloxy, (C₆-C₁₂)aryl(C₁-C₆)alkyl, heteroaryl, heteroaryl(C₁-C₆)alkyl, heterocyclic, (C₂-C₆)alkanoyl and (C₂-C₇)acyl, each of which may be unsubstituted or substituted with from 1 to 3 substituents selected from the group consisting of halogen or an isotope thereof, OH, (C₁-C₄)alkoxy, SH, NH₂, (C₁-C₄)alkylamino, di((C₁-C₄)alkyl)amino, carboxy, (C₁-C₄)alkoxycarbonyl, (C₂-C₄)alkanoyl, oxo, amido, CN, CNS, SCN, CNO, OCN and NHOH; and R³ and R⁴ are independently selected from hydrogen, (C₁-C₄)alkyl and (C₂-C₄)alkenyl, or R³ and R⁴ taken together are (C₂-C₃)alkylidine.

Typically, Y is Cl; R¹ is OCH₂CH₂¹⁸F; and R³ and R⁴ are CH₂CH₃, i.e. the compound is [¹⁸F]PBR111.

### DEFINITIONS

Throughout this specification, reference to “a” or “one” element does not exclude the plural, unless context determines otherwise.

Reference throughout this specification to “one embodiment”, “some embodiments” or “an embodiment” means that a particular feature, structure or characteristic described in connection with the embodiment is included in at least one embodiment of the present invention. Thus, appearances of the phrases “in one embodiment”, “in some embodiments” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

In the context of this specification the following terms are defined as follows:

The term “treatment” and the like in the context of the present specification refers to changes in normal TSPO-modulated physiology as well as the alleviation of the symptoms associated with a TSPO-related disease or disorder, as well as the regression and amelioration of TSPO-related diseases or disorders. The treatment may cure the disease or disorder, or delay morbidity. Hence, in the context of this invention the word “treatment” or derivations thereof when used in relation to a therapeutic application includes all aspects of a therapy, such as the alleviation of pain associated with the disease or disorder being treated, alleviation of the severity of the disease or disorder being treated, improvement in one or more symptoms of the disorder or disease being treated, improvement in the overall well-being of the subject being treated. Use of the word “treatment” or derivatives thereof will be understood to mean that the patient being “treated” may experience any one or more of the aforementioned benefits.

“Nucleic acid” as used herein includes an oligonucleotide, polynucleotide, nucleotide and fragments or portions thereof and to DNA or RNA of genomic or synthetic origin which can be single- or double-stranded, and represent the sense or antisense strand. Where “nucleic acid” is used to refer to a specific nucleic acid sequence, “nucleic acid” is meant to encompass polynucleotides that encode a specific transcript or RNA molecule that is functionally equivalent to the recited transcript of RNA molecule.

“Nucleotide” includes, but is not limited to, a monomer that includes a base linked to a sugar, such as a pyrimidine, purine or synthetic analogues thereof, or a base linked to an amino acid, as in a peptide nucleic acid (PNA). A nucleotide is one monomer in a polynucleotide. A nucleotide sequence includes the sequence of bases in a polynucleotide.

A “TSPO-associated gene product” as used herein is any gene product that is functionally linked to a TSPO gene product. The TSPO-associated gene product may be any gene product that is upstream or down-stream of any signal transduction pathway that includes a TSPO gene product. The TSPO-associated gene product may be a protein or an mRNA transcript and does not necessarily have to be immediately upstream or downstream of the TSPO gene product in a signal transduction pathway.

A “gene” refers to a DNA sequence that encodes proteins, and may or may not include introns, exons, regulatory sequences such as promoter or enhancer sequences and 5′ untranslated regions.

A “transcript”, as referred to herein, is an RNA molecule derived by the transcription of a coding gene or nucleic acid.

The term “knock-out” in relation to a gene refers to the alteration of the wild-type sequence of the gene such that no functional gene product is produced. The term “gene product” encompasses transcripts of the gene as well as proteins translated from said transcripts. For example, mutation, insertion or deletion of nucleotides of the wild-type gene sequence may lead to the entire silencing of the gene's expression, or to the expression of the altered gene sequence such that only a non-functional transcript is produced. A non-functional transcript cannot be translated into functional protein. If only one allele of the gene has been altered the gene knocked out is referred to as a “heterozygous knock-out”. If both alleles have been altered, the knock-out is referred to as a “homozygous knock-out”. In accordance with convention in the field, a heterozygous gene knock-out is indicated by “het” or “+/−” whereas a homozygous knock-out is indicated by “hom” or “−/−”. If both alleles of the gene remain unaltered (i.e. having the wild-type gene sequence) it is indicated by “wt” or “+/+”.

The terms “protein”, “polypeptide” and “peptide” mean polymers made up of amino acids linked together by peptide bonds. Unless the context indicates or requires otherwise, the terms “protein”. “polypeptide” and “peptide” are used interchangeably herein. Accordingly, for the purposes of the present invention a “polypeptide” may constitute a full-length protein or a portion of a full-length protein. A “protein”, “peptide” or “polypeptide” of the invention also encompasses naturally occurring and synthetic variants and fragments thereof.

As used herein, the term “alkyl” includes within its meaning straight and branched chain alkyl groups. Examples of such groups are methyl, ethyl, propyl, isopropyl, butyl, isobutyl, sec-butyl, ter-butyl, amyl, isoamyl, sec-amyl, 1,2-dimethylpropyl, 1,1-dimethyl-propyl, hexyl, 4-methylpentyl, 1-methylpentyl, 2-methylpentyl, 3-methylpentyl, 1,1-dimethylbutyl, 2,2-dimethylbutyl, 3,3dimethylbutyl, 1,2-dimethylbutyl, 1,3-dimethylbutyl, 1,2,2-trimethylpropyl and 1,1,2-trimethylpropyl.

As used herein, the term “cycloalkyl” refers to cyclic alkyl groups, or alkyl substituted cyclic alkyl groups. Examples of such groups include cyclopropyl, methylcyclopropyl, cyclobutyl, methylcyclobutyl, cyclopentyl, methylcyclopentyl, cyclohexyl and the like.

As used herein, the term “alkoxy” refers to a group of the formula alkyl-O—, wherein the alkyl group is as defined above.

As used herein, the term “alkenyl” includes within its meaning ethylenically mono- or di-unsaturated alkyl or cycloalkyl groups as previously defined. Examples of such alkenyl groups are vinyl allyl, 1-methylvinyl, butenyl, iso-butenyl, 3-methyl-2-butenyl, 1-pentenyl, cyclopentenyl, 1-methyl-cyclopentyl, 1-hexenyl, 3-hexenyl, cyclohexenyl, 1,3-butadienyl, 1,4-pentadienyl, 1,3-cyclopentadienyl, 1,3-hexadienyl, 1,4-hexadienyl, 1,3-cyclohexadienyl and 1,4-cyclohexadienyl.

As used herein, the term “alkynyl” includes within its meaning acetylenically unsaturated alkyl groups as previously defined. Examples of such alkynyl groups are ethynyl propynyl, n-butynyl, n-pentynyl, 3-methyl-1-butynyl, n-hexynyl and methyl-pentynyl.

As used herein, the term “alkylidene” refers to optionally unsaturated divalent alkyl radicals. Examples of such radicals are —CH₂CH₂—, —CH═CH—, —CH₂CH₂CH₂—, —CH₂CH═CH—, —(CH₂)₄—, —CH₂CH₂CH═CH—, —CH₂CH═CHCH₂— and —(CH₂)— where r is 5-7. The term also refers to optionally unsaturated divalent alkyl radicals in which one or more of the bonds of the radical from part of a cyclic system.

As used herein, the term “aryl” refers to single, polynuclear, conjugated and fused residues of aromatic hydrocarbons. Examples of such groups are phenyl, biphenyl, naphthyl, tetrahydronaphthyl, indenyl and azulenyl. Any available position of the aromatic residue can be used for attachment to the remainder of the molecule of formula (I).

As used herein, the term “aryloxy” refers to a group of the formula aryl-O—, wherein the aryl group is as defined above.

As used herein, the term “heteroaryl” refers to single, polynuclear, conjugated and fused residues of aromatic heterocyclic ring systems. Examples of such groups are pyridyl, 4-phenylpyridyl, 3-phenylpyridyl, thienyl, furyl, pyrryl, indolyl, pyridazinyl, pyrazolyl, pyralzinyl, thiazolyl, pyrimidinyl, quinolinyl, isoquinolinyl, benzofuranyl, benzothienyl, purinyl, quinazolinyl, phenazinyl, acridinyl, benzoxazolyl, benzothiazolyl and the like. Any available position of the heteroaromatic residue can be used for attachment to the remainder of the molecule of formula (I).

As used herein, the term “heterocyclic” refers to any 3- to 12-membered monocyclic, bicyclic or polycyclic ring containing, for 3- and 4-membered rings, one heteroatom; for 5-membered rings, one or two heteroatoms; for 6- and 7-membered rings, one to three heteroatoms; for 8- and 9-membered rings, from one to four heteroatoms; for 10- and 11-membered rings, from one to five heteroatoms; for 12-membered rings, from one to six heteroatoms; the heteroatom(s) being independently selected from oxygen, nitrogen and sulphur. The term “heterocyclic” includes any group in which a heterocyclic ring is fused to a benzene ring. Examples of heterocyclics are pyrryl, pyrimidinyl, quinolinyl isoquinolinyl, indolyl piperidinyl, pyridinyl, furyl, thiophenyl, tetradhyrofuryl, imidazolyl, oxazolyl, thiazolyl, pyrenyl, oxazolidinyl, isooxazolyl, isothiazolyl, isoxazolidinyl, imidazolidinyl, morpholinyl, pyrrolidinyl, pyrazolyl, pyrazolinyl, furfuryl, thienyl, benzothienyl, benzoxazolyl, benzisoxazolyl, benzothiazolyl, benzoisothiazolyl, benzothiadiazolyl, tetrazolyl, triazolyl, thiadiazolyl, benzimidazolyl, pyrrolinyl, quinuclidinyl, azanorbornyl, isoquinuclidinyl and the like. Nitrogen-containing heterocyclics may be substituted at nitrogen with an oxygen atom. Sulfur-containing heterocyclics may be substituted at sulfur with one or two oxygen atoms. Configurations of heteroatoms which result in unstable heterocyclics are not included within the scope of the definition of “heterocyclic”.

As used herein, the term “alkanoyl” refers to groups of the formula alkyl-C(O)O—, wherein the alkyl group is as defined above.

As used herein, the term “acyl” refers to any group of formula QC(O)—, wherein Q is amino, alkylamino, dialkylamino, alkyl, alkoxy, alkenyl, alkynyl, cycloalkyl, aryl, arylalkyl, heteroaryl, heteroarylalkyl and heterocyclic, each of which may be unsubstituted or substituted with from 1 to 3 substituents selected from the group consisting of halogen, OH, (C₁-C₄)alkoxy, SH, NH₂, (C₁-C₄)alkylamino, di((C₁-C₄)-alkyl)amino, carboxy, (C₁-C₄)alkoxycarbonyl, (C₂-C₄)alkanoyl, oxo, amido, CN, CNS, SCN, CNO, OCN and NHOH.

In the context of this specification, the term “comprising” means including, but not necessarily solely including. Furthermore, variations of the word “comprising”, such as “comprise” and “comprises”, have correspondingly varied meanings. Hence, the term “comprising” and variations thereof is used in an inclusive rather than exclusive meaning such that additional integers or features may optionally be present in a composition, method, etc. that is described as comprising integer A, or comprising integer A and B, etc.

The term “at least one” when used in the context of a group of sclectable elements includes any and all members of the group individually selected and includes any combination of the members of the group.

### BRIEF DESCRIPTION OF THE DRAWINGS

FIG. 1 is a schematic showing the generation of the TSPO knock-out mice. The TSPO wild-type allele consists of 4 exons. A targeting vector was created with loxP sites flanking exons 2 and 3 and a FRT-flanked neomycin cassette inserted between exons 3 and 4. Flippase recognition target (FRT) sites were also included to enable the production of conditional knock-out mice at a later stage. The neomycin cassette contains the phosphoglycerate kinase promoter (PGK) to drive expression of the neomycin resistance gene (nco), and polyadenylation signal (pA). Homologous recombination between the wild-type allele and the targeting vector generates the targeted allele in embryonic stem (ES) cells. To create conditional knock-out animals, the neomycin cassette can be removed by Flp-mediated excision to produce the floxed allele. To disrupt TSPO, cre-mediated excision is used to remove exons 2 and 3. The position of forward (FP) and reverse primers (RP1 and RP2) used for PCR genotyping are indicated. The position of the probe used for Southern blot analysis is also shown (“Probe”).

FIG. 2 shows the TSPO primer design and gel image for genotyping. The TSPO wild-type and TSPO knock-out alleles are shown in FIG. 2(a), along with the approximate locations of primer annealing sites and their subsequent PCR product length. The predicted gel electrophoresis image of PCR products from TSPO knock-out mice are shown in FIG. 2(b).

FIG. 3 shows the results obtained when genotyping putative TSPO knock-out mice. Panel (A) shows the results obtained when animals were genotyped using Southern blot analysis. An 8.8 kb fragment is expected for the wild-type allele, while a 4.2 kb fragment is expected for the knock-out allele. Panel (B) shows the results obtained when animals were genotyped by PCR. The wild-type (+/+) allele produces a 489 bp product while the knock-out (−/−) allele generates a 246 bp product.

FIG. 4 shows the radioligand membrane binding results using the TSPO-specific radioligand 3H-PK11195 to probe the renal tissue of TSPO knock-out mice.

FIG. 5 shows film autoradiographs of sections from brain, eye, heart, adrenal gland, kidney and using the probe [3H]PK11195. The organs used for film autoradiography were dissected from wild-type (TSPO^{+/+}), heterozygous (TSPO^{+/-}) and homozygous (TSPO^{-/-}) mice. The images on the left are total binding (specific and non-specific), while images on the right are non-specific bindings. The darker the image, the higher the PK11195 binding, and this is proportional to the density of TSPO. The bar graph on the top right-hand corner corresponds to the images on left.

FIG. 6 shows film autoradiographs of neuroinflammation in facial nerve nuclei that was induced by one-side facial nerve axotomy. The level of TSPO expression in heterozygous mice (middle image) was between the levels observed for wild-type and homozygous mice. The bar graph on the bottom shows the TSPO levels in the wild-type (TSPO^{+/+}), heterozygous (TSPO^{+/-}) and homozygous (TSPO^{-/-}) mice in the whole cerebellum and right side (ipsilateral side of the facial nerve axotomy) facial nucleus. Values shown in the tables below the images are the relative to those of homozygous mouse.

FIG. 7 shows the PET images of a high intensity of radiotracer ([18F]-PBR111) recorded in a wild-type mouse (TSPO^{+/+}), while none of the radiotracer was observed in the TSPO knock-out (homozygous, TSPO^{-/-}) mouse on the right. The adrenal gland is shown in the circled area.

FIG. 8 shows PET and CT images of a radiotracer ([18F]-PBR111) in a wild-type mouse (TSPO^{+/+}), and a TSPO knock-out (homozygous, TSPO^{+/-}) mouse in the bottom image. The adrenal gland is shown in the circled area. The colour from black to white represents radiotraccr intensity from low to high.

FIG. 9 shows the time course of uptake after radiotracer injection at 0 min and displacement with PBR11 at 40 min in adrenal gland, heart, kidney and liver.

FIG. 10 shows the results from the open field test with TSPO knock-out mice. Anxiety-like behaviours were measured by the lime spent in the centre and the frequency of entering the centre (A and B). Exploratory behaviours were measured by assessing time spent active (C), distance travelled (D) and non-travelling movements (E).

FIG. 11 shows the results from the emergence test with TSPO knock-out mice. Anxiety-like behaviours were measured by the time spent in the hidebox and the frequency of entering the hidebox (A and B). In addition, exploratory behaviours were also measured by assessing time spent active (C), distance travelled (D) and non-travelling movements (E).

FIG. 12 shows the results from the light/dark preference test with TSPO knock-out mice. Anxiety-like behaviours were measured by the time spent in the light compartment and the frequency of entering the light compartment (A and B). In addition, exploratory behaviours were also measured by assessing time spent active (C), distance travelled (D) and non-travelling movements (E).

FIG. 13 shows the results from the elevated plus maze test with TSPO knock-out mice. Anxiety-like behaviours were measured by the time spent in the open arms and the frequency of entering the open arms (A and B) and the time engaged in risk assessment and the frequency of risk assessment (C and D). In addition, exploratory behaviours were also measured by assessing time spent active (E) and distance travelled (F).

FIG. 14 shows the rotarod performance of TSPO knock-out mice where n=70. The time before an animal fell off the rotarod was measured.

FIG. 15, similar to FIG. 1 above, is a schematic illustrating the targeting strategy developed to generate TSPO knock-out mice according to the present invention. The TSPO wild-type allele consists of 4 exons. A targeting construct was created with loxP sites flanking exons 2 and 3 to allow for cre-mediated excision of exons 2 and 3. Homologous recombination between the wild-type allele and the targeting construct generates the TSPO targeted allele. To disrupt TSPO, i.e. to generate the TSPO knock-out allele, cre-mediated excision is used to remove exons 2 and 3. The positions of a forward primer (P1) and two alternative, reverse primers (P2 and P3) used for PCR genotyping are indicated. The position of the probe used for Southern blot analysis is also shown (“Probe”).

FIG. 16, similar to FIG. 3 above, shows the genotyping results of putative TSPO knock-out mice. Panel (A) shows the results obtained when animals were genotyped using Southern blot analysis. An 8.8 kb fragment is expected for the wild-type allele, while a 4.2 kb fragment is expected for the knock-out allele. Panel (B) shows the results obtained when animals were genotyped by PCR. The wild-type (+/+) allele produces a 489 bp product while the knock-out (−/−) allele generates a 246 bp product.

FIG. 17 illustrates the results obtained when relative TSPO mRNA expression across 13 tissues (Adrenal, Lung, Bone Marrow, Kidney, Spleen, Liver, Bladder, heart, pancreas, eye, muscle, bone and brain) was assessed. TSPO mRNA expression levels were normalised in relation to mRNA levels of general housekeeping genes Gapdh and Actb measured in the corresponding tissue.

FIG. 18 shows measurement of TSPO protein by Western blot analysis. Lysates obtained from kidney, spleen and testis of TSPO+/+, TSPO+/− and TSPO−/− mice were tested and confirmed the complete absence of TSPO protein product in TSPO^{-/-} mice (upper panel). GAPDH protein expression was used as an internal positive control (lower panel).

FIG. 19 shows images obtained using immunohistochemical antibody staining of tissue sections obtained from kidney and testis of TSPO+/+ mice (left column) and TSPO−/− mice (right column). The presence of TSPO in tissue sections of the TSPO wild-type mice and absence thereof in the TSPO knock-out mice is shown. Scale bar: 500 μm.

FIG. 20 shows images obtained using immunocytochemical antibody staining of macrophages obtained from TSPO+/+ mice (left column), TSPO+/− mice (centre column) and TSPO−/− mice (right column). The top panel shows fluorescent images of the macrophages analysing TSPO protein. The middle panel shows fluorescent mitochondrial staining of the macrophages (mitochondrial electron transport chain complex IV). In the bottom panel, the corresponding images of the top and middle panel have been merged showing the predominant localization of TSPO protein in the mitochondria. The gradual reduction of TSPO protein from wild-type to homozygous knock-out visible in the top panel as well as the overlap between TSPO protein with mitochondrial staining validates mitochondria as the primary site of the TSPO protein, which is shown to be entirely absent in TSPO^{-/-} mice. No obvious difference in intracellular density or distribution of the mitochondria was detected in the TSPO^{-/-} mice. Scale bar: 20 μm.

FIG. 21 illustrates that no constitutive or inducible TSPO ligand binding occurs in TSPO^{-/-} mice. FIGS. 21a and b show the chemical structures of two TSPO-binding ligands, PK1195 and CLINDE/PBR111, respectively. FIG. 21c is a schematic illustrating the axotomy of the facial nerve. FIG. 21d shows film-autoradiography with [³H]PK11195 and [¹²⁵I]CLINDE and immunohistochemical staining of themicroglial activation markeranti-CD11b on consecutive brain sections confirmed the previously reported localised induction of TSPO ligand binding in the injured facial nucleus contemporaneous to the activation of microglia of TSPO^{+/+} animals. In contrast, FIG. 21e shows that no binding of [³H]PK11195 and [¹²⁵I]CLINDE was induced in TSPO^{-/-} mice despite the presence of activated microglia in the injured facial nucleus, thus providing evidence that the high selectivity of [³H]PK11195 and [¹²⁵I]CLINDE for their respective binding sites on the TSPO is retained in pathologically changed tissue. FIG. 21f shows the immunofluorescent anti-CD11b staining of activated microglia in the injured facial nucleus of TSPO^{-/-} mice. The images reveal that the TSPO knock-out mice show no obvious differences from the well-studied normal appearance of microglial activation in wild-type animals, i.e. they also show the characteristic localisation of activated, perineuronal microglia. Star denotes the soma of a neuron. FIG. 21g shows the characteristic localisation of activated, perineuronal microglia seen in FIG. 21f at a higher magnification. Scale bar in 100 μm. Star denotes the some of a neuron. FIG. 21h illustrates that TSPO^{+/+}, TSPO^{+/-} and TSPO^{-/-} mice were identical in external appearance (top panel). Whereas the lower panel shows in vivo images obtained with PET/CT using the radioligand [¹⁸F]PBR111 (the ¹⁸F-labelled analogue to [¹²⁵I]CLINDE), which strikingly illustrate that TSPO^{-/-} mice show no ligand binding (thus also demonstrating the selectivity of the used ligand). Occasional signals originating from the excretory pathways, such as from the gut and urinary bladder. However, both TSPO^{+/+} and TSPO^{+/-} mice show similar distribution of ligand binding, i.e. in organs with known high TSPO expression, notably kidney and adrenal gland (circle). The images are displayed with gradual scaling and are directly comparable (highest values are white). FIG. 21i, The kinetics of [¹⁸F]PBR111, and the displacement of the radioligand after injection (indicated by arrows) with cold PBR111 (1 mM) to establish non-specific binding, demonstrated that TSPO^{-/-} mice do not have specific binding of [¹⁸F]PBR111 in any organs while the ligand kinetic in TSPO^{+/+} and TSPO^{+/-} mice indicates specific binding. (ID=injected dose; error bars denote standard deviation).

FIG. 22 illustrates the functional effects of the global TSPO knock-out according to the present invention. FIG. 22a to c show that no significant differences between TSPO^{+/+} and TSPO^{-/-} mice were found in blood pregnenolone concentrations (a), mRNA expression of steroidogenic acute regulatory (StAR) protein, P450Scc (CYP11A1), and TSPO2 (normalised to house-keeping genes Actb and Gapdh) (n=3 per genotype) (b) and active unit (A.U.) of protoporphyrin IX (PPIX)/ml blood (c) (left: typical spectrum after addition of 5-aminolevulinic acid (ALA), right: no significant differences in levels of PPIX) (n=3-5 per genotype). FIG. 22d illustrates that TSPO^{+/+} and TSPO^{-/-} animals showed no difference in food and water intake under either the control or high fat diet (n=4 per genotype). FIG. 22e shows that TSPO^{-/-} animals responded to high fat diet compared to control diet with a non-significant relative weight gain while wild-type animals had the expected significant increase in relative weight gain (p<0.05)(n=4 per genotype). FIG. 22f illustrates the comparison of the normalised weight development across the entire 10 week period showing the significantly different weight development. (n=4 per genotype). FIG. 22g illustrates that, while TSPO^{+/+} mice showed the expected trend towards glucose intolerance under the high fat diet compared to the control diet, TSPO^{-/-} mice showed a near identical response to glucose injection under either diet regimen (n=4 per genotype).

FIG. 23 illustrates altered Mitochondrial Energy Metabolism in TSPO^{-/-} mice according to the present invention. FIG. 23a is a schematic illustration of the mitochondrial electron transport chain (ETC). FIGS. 23b and 23c are bar graphs illustrating that the basal oxygen consumption (OCR; b) and extracellular acidification rates (ECAR; c) in microglia (a cell type rich in mitochondria) from TSPO^{-/-} mice was significantly lower than in wild-type microglia. FIGS. 23d and 23e show measurements of OCR (d) and ECAR (e) from microglia of TSPO^{-/-} and TSPO+/+ mice which illustrate that inhibition ofATP synthase with oligomycin (3 μM) led to significantly reduced ATP production in microglia of TSPO^{-/-} mice compared to the ATP production induced in microglia of TSPO^{+/+} mice. FIGS. 23f and 23g show measurements of OCR (f) and ECAR (g) from microglia of TSPO^{-/-} and TSPO+/+ mice using the uncoupling agent FCCP (0.1 μM). The data shown confirm the significant reduction in maximal (reserve) respiration of OCR and ECAR seen in TSPO^{-/-} microglia compared with the wild-type.

FIGS. 23h and 23i show measurements of OCR (h) and ECAR (i) from microglia of TSPO−/− and TSPO+/+ mice. While the total respiratory capacity of mitochondria inhibited with rotenone and antimycin A showed similar OCR in TSPO+/+ and TSPO−/−, ECAR was significantly reduced in TSPO−/− microglia. FIG. 23j illustrates the combined data of the effect of inhibitors of the electron transport chain or decoupling agents (see FIGS. 23d, f and h) show that the proton leak in TSPO−/− microglia was significantly greater than in TSPO+/+ microglia (*, p<0.05, n=14 wells for TSPO+/+ and n=10 wells for TSPO−/− microglia).

FIG. 24 illustrates the data obtained from Quantification of [¹⁸F]PBR111 positron emission tomography in tissues of TSPO+/+. TSPO+/− and TSPO−/− mice. Adrenal gland tissue (a), kidney (b), heart (c), liver (d), lung (e) and brain (f). Time-activity curves of [¹⁸F]PBR11 until displacement at 40 mins with cold PBR111 (1 mM) indicated by arrows.

FIG. 25 illustrates TSPO protein expression revealed by autoradiography and radioligand binding. Receptor autoradiography of 16 μm sections from TSPO^{-/}, TSPO^{+/-} and TSPO^{-/-} mice using [³H]PK11195 (a) and (b) [¹²⁵I]CLINDE on spleen sections, [³H]PK11195 (C) and [¹²⁵I]CLINDE (d) on kidney sections, and [³H]PK11195 (e) and [¹²⁵I]CLINDE (f) on testis sections. Total binding is given as well as competitive binding with 10 μM unlabelled CLINDE (CB(CL)), PK11195 (CB(PK)) and PBR-111 (CB(PBR)). Specific binding of 3 nM [¹²⁵I]CLINDE and 1 nM [³H]PK11195 is clearly visible in tissue sections from TSPO^{+/+} and TSPO^{+/-} mice and is displaced by all 3 unlabelled ligands, whereas the binding in TSPO^{-/-} tissue is at background level and not displaceable. Specific binding using [³H]PK11195 is shown in testicular tissue (g) and kidney tissue (h). In both tissues, compared to TSPO^{+/+} mice, TSPO^{+/-} mice had approximately half the signal and TSPO^{-/-} mice had close to zero signal. Dotted lines represent non-linear regression of experimentally obtained data points and data are expressed as percentage relative to TSPO^{+/-} specific binding.

### DETAILED DESCRIPTION OF THE PREFERRED EMBODIMENTS

The present inventors have surprisingly, and against published reports and prevailing view, been able to generate a viable transgenic non-human animal that is lacking a functional TSPO gene. The invention may be used as a negative control for diagnosis and imaging purposes and to investigate biologically active agents useful in the treatment of TSPO-related disorders.

Accordingly, the present invention provides a transgenic non-human animal, comprising cells with at least one copy of a non-functional, endogenous TSPO gene.

Transgenic Animals

The animal may be any animal capable of being bred to produce viable, offspring with cells comprising at least one copy of a non-functional endogenous TSPO gene product. The animal may, for example, be a mammal, including but not limited to a mouse, a rat, a sheep, a dog, a cow, a horse, a non-human primate, a pig, a cat, a rabbit, a goat, a ferret, a guinea pig, a gerbil or a hamster. The animal may also be a bird, including, but not limited, to a chicken, a duck or a quail. The animal may be an insect, including, but not limited to, flies such as those belonging to the Drosophila family, such as Drosophila melanogaster. The animal may also be a fish, including, but not limited to, those belonging to the Cyprinidae family. For example, the fish may be a zebra fish or a medaka fish. The animal may also be a leech belonging to the Hirundinea family.

In one embodiment of the present invention, the animal is a member of the Murine family. In a further embodiment, the animal is a mouse.

A transgenic animal may, for example, be any animal with a genome that has been stably and deliberately modified that is capable of transmitting this modification to progeny. In general, a transgenic animal will have a genome that comprises some foreign DNA, wherein foreign DNA is any DNA sequence that would not be present in the genome of said transgenic animal if not for the deliberate modification made to said genome. The foreign DNA sequence may, for example, correspond to all or part of one or more genes, all or part of an exon, a promoter region, and enhancer sequence, a consensus sequence, a STOP codon, a START codon, a selection marker, a fluorescence tag, or a homologous recombination site.

It would be understood that the modifications to the genome are transmitted through the germ lines of transgenic animals such that every cell in the transgenic animal comprise the same modified genetic material. It will also be understood that a foreign DNA sequence, once incorporated into the genome of a transgenic animal such that it is transmitted through the germ lines of said transgenic animal, may also be an endogenous DNA sequence.

A non-functional endogenous TSPO gene is one that, for example, does not produce TSPO gene product in a cell or produces a non-functional TSPO gene product in a cell. A TSPO gene product is the product of TSPO gene expression and may be, for example, the product of transcription of a TSPO gene or translation of a TSPO mRNA transcript. A non-functional TSPO gene product is one that is in some manner different to a normal (control) TSPO gene product, and may, for example, be a non-functional mRNA transcript (before or after post-transcriptional modification), or a non-functional polypeptide (before or after post-translational modification).

A person of skill, using freely accessible sequence databases, is able to determine what constitutes a normal (control) TSPO gene product. For example, the NIH genetic sequence database, Genbank (http://www.ncbi.nlm.nih.gov/genbank) would provide information on the expected molecular weights of normal TSPO mRNA transcripts (before and after post-transcriptional modification), exon/intron splicing boundaries, and the expected molecular weight of a normal TSPO polypeptide.

A non-functional TSPO mRNA transcript is an mRNA that cannot be translated to produce a normal TSPO polypeptide. For example, a non-functional TSPO mRNA transcript may be one that comprises a non-functional 3′ untranslated region that prevents translation of the mRNA transcript or may undergo incorrect splicing due to abnormal exon/intron boundaries of the TSPO mRNA transcript. In another example, a non-functional TSPO mRNA transcript may produce no TSPO polypeptide. In a further example, a non-functional TSPO mRNA transcript may comprise a deletion of one or more exons, thus producing no TSPO polypeptide or a truncated TSPO polypeptide.

A non-functional TSPO polypeptide is a polypeptide that cannot perform all or some of the functions of a normal TSPO polypeptide in a cell or a control TSPO polypeptide. For example, a non-functional TSPO polypeptide may be one that does not fold correctly after expression. Non-limiting examples of a non-functional TSPO polypeptide include one with (1) one or more substitutions in the TSPO polypeptide such that one or more of the amino acid residues are different to the normal TSPO polypeptide; (2) one or more deletions of one or more amino acid residues from the TSPO polypeptide; (3) one or more additions of one or more amino acid residues to the sequence of the TSPO polypeptide; and (4) a C-terminal or N-terminal truncation of one or more amino acids.

It would be understood that cells with only one copy of a non-functional endogenous TSPO gene may have one copy of a functional endogenous TSPO gene, which may produce a functional TSPO gene product.

In another embodiment of the present invention, the transgenic animal may comprise cells with two copies of a non-functional endogenous TSPO gene. These cells would not be capable of producing a functional TSPO protein product. A person of skill in the art would understand that a cell with one copy of the non-functioning TSPO gene would express different levels of a functional TSPO protein product when compared to a wild-type cell and a cell with two copies of a non-functioning TSPO gene.

In one embodiment of the present invention, the non-functional endogenous TSPO gene contains at least one mutation that prevents the TSPO gene from producing a normal TSPO gene product or any TSPO gene product. The mutation may be, for example, a deletion or an insertion of one or more nucleotides, a frame-shift mutation, re-arrangement or a substitution of one or more nucleotides. For example, the deletion may be of one or more exons, or the mutation may be a substitution of part of, or all of, the TSPO promoter region with a nonsense sequence. The mutation may occur, for example, in an exon, intron or an intron/exon splice-site. The mutation may occur in the 5′ transcription initiation region upstream of the gene or occur in the region corresponding to the 3′ untranslated region of the mRNA transcript. A deletion may, for example, occur concurrently with an insertion in the event of a recombination event, and the insertion may comprise more than, less than, or the same number of nucleotides as the deletion.

The non-functional endogenous TSPO gene may contain more than one mutation and these mutations may be the same type or different types. The mutations may occur on different regions of the gene. For example, the TSPO gene may contain a frame-shift mutation in an exon as well as a deletion or one or more nucleotides. In another example, the TSPO gene may comprise one or more insertions of one or more nucleotides that comprise a recombination consensus sequence. In a further example, the TSPO gene may comprise at least two insertions of one of more nucleotides that comprise a recombination consensus sequence, as well as a deletion of one of more nucleotides on either side of said insertions.

In one embodiment, the non-functional endogenous TSPO gene contains a mutation that comprises a deletion of all or part of exon 1, 2, 3 or 4 of the TSPO gene. In a further embodiment, the non-functional endogenous TSPO gene contains a mutation that comprises a deletion of all or part of exon 2 or exon 3 of the TSPO gene. For example, the mutation may comprise a deletion of all or part of exon 2, all or part of exon 3, or all or part of exon 2 and 3. Typically, the mutation is a deletion of exons 2 and 3. In another example, the mutation may comprise a deletion of all or part of only exon 1, all or part of exon 2, or all or part of exon 1 and 2. A deletion of all or part of exon 1, 2, 3 or 4 is any deletion that may prevent the TSPO gene from producing a TSPO gene product or a normal TSPO gene product.

The skilled person would be aware of the methods that may be used to generate the transgenic animals of the present invention. In general, the methods rely on gene disruption via the incorporation of artificial DNA into pluripotent cells, such as, for example, embryonic stem (ES) cells, or embryonic cells at various stages of development. The method used to introduce the artificial DNA may depend on the stage of development of the embryonic cells. ES cells that have been transfected with artificial DNA can be used to colonise embryos and generate founder, transgenic animals.

Founder animals can be bred, inbred, outbred, or crossbred to produce colonies of the particular animal. The transgenic animals are screened and evaluated to select those animals having the genotype of interest. Initial screening can be performed using, for example, Southern blot analysis or PCR techniques to analyse animal tissues to verify the presence of at least one copy of a non-functional TSPO gene in the genome of the animal. The level of mRNA expression of the TSPO gene in various tissues of the transgenic animals can also be assessed using techniques which include, but are not limited to, Northern blot analysis of tissue samples obtained from the animal, in situ hybridization analysis, and reverse transcriptase-PCR (rt-PCR). The level of TSPO protein expression in tissues can be determined by common immunological techniques using antibodies specific for TSPO, or receptor binding assays using TSPO—binding compounds or ligands.

Non-limiting examples of methods that may be used for the introduction of the artificial DNA comprising a TSPO gene with one of more mutations into an embryonic stem cell include homologous recombination and gene trapping. Homologous recombination, with the loxP-Cre recombinase system and artificial DNA constructs, is commonly used to generate transgenic animals, and in particular, transgenic animals that belong to the murine family. In particular embodiments of the present invention, the transgenic animal is generated using conventional Cre/lox breeding methods. For example, transgenic animal comprising cells with at least one copy of a non-functional endogenous TSPO gene comprising a mutation that is a deletion of exon 2 or exon 3 may be generated using homologous recombination. For example, the generation of a transgenic mouse with a genome that is homozygous for a TSPO gene with deletion of exons 2 and 3 may comprise the step of cross-breeding a transgenic mouse with a genome comprising a foreign Cre recombinase gene, with a transgenic mouse with a genome that is homozygous for a TSPO gene comprising two loxP sites which flank exon 2 and 3 of the TSPO gene.

In one embodiment of the invention, the non-functional endogenous TSPO gene comprises a constitutive mutation. By constitutive mutation is meant a mutation in the TSPO gene that prevents the TSPO gene from producing a TSPO gene product or a normal TSPO gene product in every cell of the transgenic animal. In another embodiment of the invention, the TSPO gene contains a mutation that is conditional. A conditional mutation is one that prevents the TSPO gene from producing a TSPO gene product or a normal TSPO gene product, for example, in only selected cells of the transgenic animal or at only selected times during the transgenic animal's development.

A person of skill would understand that the conventional methods used to generate transgenic animals, such as, for example, the loxP-Cre recombinase system, can be manipulated such that only specific cells and/or tissues, or cells and/or tissues at specific times, comprise non-functional genes. Appropriate DNA constructs used for the generation of transgenic animals can be engineered to be operatively linked to regulatory elements, such as promoters or enhancers, which are only active in specific cell types or at specific times. The term “operatively linked” means that a DNA sequence and a regulatory sequence/(s) (i.e. promoters) are connected in such a way as to permit gene expression when the appropriate molecules (i.e. transcription factors) are bound to the regulatory sequence/(s). The use of DNA constructs that are operatively linked to regulatory elements allows for the targeted expression of a gene of interest. A person skilled in the art can readily determine an appropriate promoter or enhancer that allows expression of an artificial DNA construct, for example, in a desired cell and/or tissue or at a desired time. Cell/tissue-specific or time-specific (i.e. temporal) expression does not require a complete absence of expression in cells and/or tissues other than the preferred cell and/or tissue, or a complete absence of expression at times other that then preferred time. Instead, “cell-specific” or “tissue-specific” or “time-specific” expression refers to a majority of the expression of a particular DNA sequence in the preferred cell type or tissue or at the preferred time.

For example, the generation of a transgenic mouse of the present invention may comprise the step of cross-breeding a transgenic mouse comprising an exogenous Cre recombinase gene that is controlled by a kidney cell-specific promoter, with a transgenic mouse with a TSPO gene comprising two loxP site insertions, which flank exon 2 of the TSPO gene. In this instance, every cell of the resulting transgenic mouse may comprise the same genetic material. The genetic material of each mouse will include foreign DNA comprising the Cre recombinase gene, as well as the TSPO gene comprising the two loxP site insertions. The Cre recombinase may only be expressed in the kidney cells that express the appropriate transcription factors to initiate expression of the Cre recombinase from the kidney cell-specific promoter. Therefore, the kidney cells may be the only cells that comprise at least one copy of a non-functional, endogenous gene with a mutation that prevents the TSPO gene from producing a TSPO gene product or a normal TSPO gene product, wherein the mutation is the deletion of exon 2 of the TSPO gene.

In a further non-limiting example, the generation of a transgenic mouse of the present invention may comprise the step of first cross-breeding a transgenic mouse comprising an exogenous Cre recombinase gene that is controlled by a temporal promoter (for example, one that is only activated when the animal reaches adulthood), with a transgenic mouse with a modified endogenous TSPO gene comprising two loxP site insertions, which flank the transcription initiation region of the TSPO gene. In this instance, every cell of the resulting transgenic mouse may comprise the same genetic material. The genetic material of each mouse will include foreign DNA comprising the Cre recombinase gene, as well as the TSPO gene comprising the two loxP site insertions. The Cre recombinase may only be expressed in the cells when the mouse reaches adulthood and the appropriate transcription factors to initiate expression of the Cre recombinase are present in the cells. Therefore, only when the mouse reaches adulthood may the cells in the adult mouse comprise at least one copy of a non-functional, endogenous gene with a mutation that prevents the TSPO gene from producing a TSPO gene product or a normal TSPO gene product, wherein the mutation is the deletion of the transcription initiation region of the TSPO gene.

It would be understood that mice that are homozygous for any of the desired mutations may be generated by cross-breeding mice that are heterozygous for the desired mutation though standard methods.

In one embodiment, the present invention relates to the progeny of any of the herein described transgenic, non-human animals comprising cells with at least one copy of a non-functional, endogenous TSPO gene.

The progeny may be from any breeding generation. For example, the progeny may be from a non-human transgenic animal that is a third generation non-human transgenic animal. The resulting progeny may, for example, comprise one or two copies of the non-functional endogenous TSPO gene. The resulting progeny may, for example, be heterozygous or homozygous for any of the genotypes prescribed by the breeding pair.

The progeny may be generated from breeding a non-human animal of the present invention with any other animal of the same species. For example, the progeny may be generated from breeding a non-human animal of the present invention with a transgenic animal of the same species with a different genetic modification. In another example, the progeny may be generated by breeding a non-human animal of the present invention with any wild-type animal of the same species.

In a further embodiment, the present invention relates to the progeny generated from breeding any of the herein described transgenic mice comprising cells with at least one copy of a non-functional endogenous TSPO gene, including but not limited to a pKZ1 mouse, a Brca2 homozygous mouse, a Tg(CAT)(+/+) mouse, an A-T mutated heterozygous/homozygous mouse, a Csbm/m mouse, an insulin-like growth factor 1 heterozygous and homozygous mouse, a P53 heterozygous and homozygous mouse, a radiation sensitive and resistant transgenic mouse, a Schizophrenia DISC1 knock-out mouse, a Schizophrenia neuregulin 1 knock-out mouse, a genetic engineering model tumour mouse, a neuroinflammation model mouse, an Alzheimer's Disease model mouse, a Parkinson's disease model mouse, or a mouse with targeted deletion of the type 2 deiodinase gene (D2KO) that is insulin resistant and susceptible to diet induced obesity.

Methods and Uses Related to the Transgenic Non-Human Animals

The transgenic non-human animals of the present invention are particularly useful in in vivo studies relating to TSPO function and TSPO-related disorders and the study of TSPO-associated gene products or compounds interacting directly or indirectly with the TSPO. However, the animals may also be used for in vitro studies of TSPO function and TSPO-related disorders. A TSPO-associated gene product is any gene product that is functionally linked to a TSPO gene product. The TSPO-associated gene product may be any gene product that is upstream or down-stream of any signal transduction pathway that includes a TSPO gene product. The TSPO-associated gene product may be a protein or an mRNA transcript and does not necessarily have to be immediately upstream or downstream of the TSPO gene product in a signal transduction pathway. For example, a TSPO-associated gene product may be a transcription factor that binds to the transcription initiation region of the TSPO gene and initiates transcription. In another example, a TSPO-associated gene product may be a protein that is expressed in response to TSPO-mediated steroid production in a cell.

In one embodiment, the present invention relates to the cells, tissues and immortalised cells lines derived from any transgenic non-human animal comprising cells with at least one copy of a non-functional, endogenous TSPO gene described herein. The present invention also relates to the cells, tissues and immortalised cell lines derived from the progeny of any of the transgenic non-human animal comprising cells with at least one copy of a non-functional, endogenous TSPO gene described herein.

The cells or immortalised cell lines may be any cells extracted, obtained and/or derived from any animal, including, but not limited to, germ cells, stem cells, nerve cells, sensory cells, endothelial cells, cardiac cells, smooth muscle cells, osteoblasts, melanocytes, hepatic cells, renal cells, adrenal cells, haematopoietic cells and adipocytes.

A person of skill would know how to generate immortalised cells from cells and tissues obtained from an animal.

The tissues may be any tissues obtained from any animal including, but not limited, brain tissue, kidney tissue, lung tissue, heart tissue, adrenal gland tissue and gonad tissue. The tissue may, for example, be healthy tissue or may comprise malignancies.

The tissues and cells may be obtained or extracted from any biological sample taken from any of the animals of the present invention described herein. Non-limiting examples of biological samples include sections of tissues such as biopsy and autopsy samples, sections taken for histological purposes, such as frozen sections, blood, plasma, serum, sputum, stool, tears, mucus, hair, and skin. Biological samples also include lymph fluid, ascetic fluid. Biological samples also include archival samples, such as those having treatment or outcome history.

The biological sample may be obtained and used immediately or may be stored under appropriate conditions prior to use. For particular embodiments, appropriate conditions are those which permit storage for a desired period of time under conditions which substantially prevent or retard the degradation of a TSPO gene product or a TSPO-associated gene product. One or more additional components may be included with the biological sample. Examples of additional components which may be included are protease inhibitors to inhibit the degradation of proteinaceous components of the biological sample, RNase inhibitors or DNase inhibitors to inhibit the degradation of nucleic acid components of the biological sample and preservatives or other anti-bacterial components to minimize bacterial contamination of the sample. Additional components may be added during collection or subsequent to collection, before or after storage, and may remain during use of the biological sample for detection or analysis of a TSPO gene product or TSPO-associated gene product, or may be removed or inactivated prior to use of the biological sample for detection or analysis of a TSPO gene product or TSPO-associated gene product.

The animals, progeny, cells, tissues and immortalised cell lines of the present invention are useful for studying and analysing TSPO-related diseases or disorders, including altered interactions of the TSPO with other compounds or proteins. A TSPO-related disorder may be any disorder that is characterised by perturbance of normal TSPO expression or normal TSPO function in a subject. The characteristics associated with normal TSPO function or expression can be found in a control. By way of example, where abnormal TSPO function or expression is associated with a disease condition, appropriate controls indicative of normal TSPO function or expression might include an individual who is not suffering from a TSPO-related disorder or a population standard of individuals believed not to be suffering from a TSPO-related disorder. Controls appropriate for the determination of normal TSPO function may include laboratory standards or values based on known or determined population standards or values, and may be supplied in the format of graphs or tables that permit easy comparison of measured, experimentally determined values.

Non-limiting examples of TSPO-related diseases and disorders include cancer, neuroinflammation, Alzheimer disease, Parkinson's disease, Epilepsy, brain injury, Ischemia-reperfusion injury, behaviour or neurological or psychiatric disorders including acute and chronic stress, anxiety disorders, mode disorders, and Schizophrenia, peripheral neuropathy, Multiple sclerosis, neuropathic pain, obesity, diabetes and cachexia.

A subject may have a hereditary, acquired, induced or temporary TSPO-related disease or disorder. As such, a subject may be diagnosed as having a TSPO-related disease or disorder at any particular stage in their lifetime, regardless of whether said subject had previously been diagnosed as not having a TSPO-related disease or disorder.

The animals, progeny, cells, tissues and immortalised cell lines of the present invention are particularly useful for methods relating to the imaging of TSPO gene products in biological samples from animals, as well detecting TSPO gene products in biological samples from animals and detecting TSPO-associated gene products.

In the methods of the present invention, by “detecting”, or “detection”, is meant determining, or the determination of, the presence, absence, activity or amount of a gene product in a sample, and may include quantifying the amount of gene product in a sample or quantifying the activity of a gene product in a biological sample. Quantifying, and hence detecting, includes relative quantification, such as where a given test sample is assessed for the presence of a TSPO gene product by comparison with a control sample, and the test sample is found to comprise more than, less than or about the same amount of TSPO gene product as the control sample. In this instance, a biological sample from an animal of the present invention that is homozygous for a non-functional, endogenous TSPO gene could be used as a negative control sample. Furthermore, a biological sample from an animal that is heterozygous for a non-functional, endogenous TSPO gene could be used as a control sample that demonstrates lower expression levels of a TSPO gene product than a wild-type animal or reveals not only quantitatively but also qualitatively different pharmacokinetic or pharmacodynamics differences due to the loss of one functional TSPO allele.

Detecting also includes comparison of a TSPO gene product in a sample with another analyte detectable in the sample. For example, the amount of TSPO gene product in a given test sample may be quantified relative to an internal reference marker. Detecting also includes absolute quantification such that the amount of a TSPO gene product in a sample may be determined and expressed in appropriate units, for example, as units/volume of sample, such as grams, micrograms, nanograms, picograms, femtograms, and the like, per millilitre, microlitre, nanolitre and the like. The skilled addressee will understand that in detecting a TSPO gene product in a test sample, physical characteristics of the TSPO gene product, such as molecular weight, binding capacity with respect to nucleic acid probes, antibodies or natural and/or synthetic compounds, may also be concurrently determined.

It will be understood that the preparation of a biological sample for use in the methods of the present invention may be performed by any suitable means, which may depend on the TSPO gene product intended to be imaged or detected and the detection or imaging technique to be used. The TSPO gene product may be any appropriate gene product, including, but not limited to, nucleic acids, for example mRNA or cDNA transcripts and the like, and TSPO polypeptides, and fragments thereof.

Where detection or imaging or functional testing of a TSPO polypeptide or fragment thereof is desired, the sample may be prepared by any suitable means for analysis of one or more polypeptides in a sample, including, for example, crude homogenates of samples, extraction of total protein from the sample, extraction of phosphorylated protein from the sample, preparation of polypeptide fragments from the sample, preparation of cryosections from the sample, and/or preparation of fixed cells or tissue section from the sample. TSPO polypeptides or fragments thereof can be imaged or detected by established protein assay and imaging methods, including, but not limited to (i) immunoassay methods involving binding of an antibody or probe to the TSPO polypeptide or a fragment thereof; and (ii) proteomic and mass spectrometry methods.

Immunoassay techniques and protocols are generally described in Price and Newman, “Principles and Practice of Immunoassay.” 2nd Edition, (Grove's Dictionaries, 1997); and Gosling. “Immunoassays: A Practical Approach,” (Oxford University Press, 2000). Non-limiting immunoassay methods that will be known to a skilled addressee include western blots, enzyme-linked immunosorbent assay (ELISA), IgM antibody capture ELISA (MAC ELISA), immunohistochemistry (IHC), and immunocytochemistry (ICC). If desired, such immunoassays can be automated. Immunoassays can also be used in conjunction with laser induced fluorescence, for example flow cytometry.

Proteomic and mass spectrometry methods known in the art are described in “Methods in Molecular Biology” (Volume 428 Clinical Proteomics; Methods and Protocols; Vlahou, Antonia 2008). Useful proteomic assays include any proteomic assay as known in the art such as, but not limited to, two-dimensional gel electrophoresis, mass spectrometry (MS), tandem mass spectrometry and multiple rounds of mass spectrometry, and receptor membrane binding methods, including use labelled or label-free methods, such a plasmon-resonance techniques.

The antibodies and probes used in the methods of the present invention may be derived from any source. The antibodies may be from any animal origin and may, for example, be monoclonal, polyclonal, chimeric, multispecific, humanized, and human monoclonal and polyclonal antibodies which specifically bind the TSPO polypeptide of fragments thereof. The antibodies or probes may be commercially available or may be specifically generated for use in the methods of the invention. For example, the probe used to detect a TSPO gene product may be the TSPO-specific probe PK11195. The probe may also be the TSPO-specific radiolabelled probe [³H]PK11195. Methods for the generation of suitable antibodies or probes will be readily apparent to those skilled in the art. For example, a monoclonal antibody specific for a target molecule of interest, typically containing Fab portions, may be prepared using the hybridoma technology described in Harlow and Lane (eds.), (1988), “Antibodies-A Laboratory Manual”, Cold Spring Harbor Laboratory, N.Y.

Where detection or imaging or functional testing of a TSPO mRNA transcript or fragment thereof is desired, the sample may be prepared by any suitable means for analysis of one or more nucleic acids in a sample, including, for example, extraction of total RNA from the sample, extraction of polyA⁺ RNA from the sample, and/or preparation of cDNA representative of expression of messenger RNA within the sample. The cDNA preparation may be a total cDNA preparation, such that it represents a library of all or substantially all mRNA species in the sample or the cDNA preparation may be prepared in such a way that it is enriched for the inclusion of particular species, such as one or more markers of interest for a given analysis.

Methods for the detection or imaging of TSPO mRNA transcripts or fragments thereof can be performed, for example, by hybridization of mRNA, or an amplified or cloned version thereof from a biological sample to a polynucleotide that is unique to all or part of a TSPO gene sequence. Suitable nucleic acid detection methods employing labelled probes are well known in the art and include but are not limited to, RNase Protection Assays, fluorescence in situ hybridization (FISH), in situ hybridization, Northern, South-Western, North-western and Southern blotting or differential display.

A probe may be attached to a detectable label or other reporter molecule. Non-limiting examples of typical labels include radioactive isotopes, enzyme substrates, cofactors, ligands, chemiluminescent or fluorescent agents, haptens, and enzymes. Methods for labelling and guidance in the choice of labels appropriate for various purposes are discussed in, for example, Sambrook et al. (In Molecular Cloning: A Laboratory Manual, CSHL, New York, 1989) and Ausubel et al. (In Current Protocols in Molecular Biology, John Wiley & Sons, New York, 1998).

Methods for preparing and using nucleic acid probes are described, for example, in Sambrook et al. (In Molecular Cloning: A Laboratory Manual, CSHL New York, 1989). Ausubel et al. (ed.) (In Current Protocols in Molecular Biology, John Wiley & Sons, New York, 1998), and Innis et al. (PCR Protocols, A Guide to Methods and Applications, Academic Press, Inc., San Diego, Calif., 1990).

In one embodiment, the animals, progeny, cells, tissues and/or immortalised cell lines of the present invention as described herein are used as a negative control for the detection of a TSPO gene product or for analysis of selective TSPO-mediated actions or adaptive responses due to the absence of one or more functioning TSPO allele in a biological sample.

In particular embodiments, the biological samples are from subjects that have or are suspected to have a TSPO-related disorder.

The use of the animals, progeny, cells, tissues and/or immortalised cell lines of the present invention as negative controls in assays and experiments based on the detection, imaging or functional studies of TSPO or TSPO-associated gene products may assist in the diagnosis of TSPO-related diseases and disorders.

It will be understood that the term “diagnosis” includes distinguishing between having and not having a TSPO-related disorder at a given time, as well as distinguishing between having and not having an increased risk of developing a TSPO-related disorder at any time during the lifetime of the subject. For instance, abnormal TSPO gene expression can be said to be associated with TSPO-related diseases and disorders and a tendency to develop TSPO-related diseases and disorders. The animals, progeny, cells, tissues and/or immortalised cell lines of the present invention are used as negative controls in any other tests related to TSPO-related diseases and disorders.

The non-human animals, tissues, cells and immortalised cell lines of the present invention as herein described may also be useful for the identification and screening of candidate compounds for use in the treatment of a TSPO-related disease or disorder in a subject.

Accordingly, candidate compounds may include compounds with an established or suspected binding affinity to TSPO gene products or TSPO-associated gene products. Non-limiting examples of candidate compounds include PK11195, Ro54864, PBR111 and CLINDE.

To be useful for the treatment of a TSPO-related disease or disorder, candidate compounds generally require characterisation in regard to their specificity and selectivity for an intended target, such as TSPO or TSPO-associated gene products. The distinction of selective or specific binding or interactions from non-selective and non-specific binding or interactions with other targets can be firmly established in a non-human animal, tissue, cell or immortalised cell line model according to the present invention, i.e. in a model where the intended target is not present, or is expressed at lower levels than in a wild-type model Research has shown that TSPO is highly conserved across most species, which allows for meaningful information to be derived from studies with non-human animals to be applied to TSPO-related diseases and disorders in humans and other subjects.

With respect to the candidate compounds, the binding selectivity is a measure of the affinity with which the compound binds a particular target over another target. Binding specificity may be expressed numerically as selectivity co-efficient. Binding specificity relates to the way in which a candidate compound may bind a target molecule.

Accordingly, the non-human animals, tissues, cells and immortalised cell lines of the present invention as herein described may be used in a variety of screening and identification assays. For example, a variety of compounds suspected of affecting TSPO expression and activity, or the expression and activity of TSPO-associated gene products, have been screened and identified as being useful in the treatment of a TSPO-related disease and disorder in a subject.

In certain, non-limiting embodiments of the present invention, compounds useful in the treatment of a TSPO-related disease or disorder is a compound of general formula (I), as disclosed in U.S. Pat. No. 6,379,649 (the disclosure of which is herewith incorporated by reference):

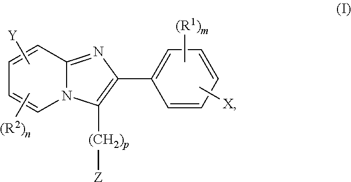

wherein

X is absent, iodine or an isotope thereof;

Y is selected from F, Cl, Br, I, OH, SH, NH₂, CN and COOH;

Z is selected from N(R³)C(O)R⁴ and C(O)NR³R⁴;

R¹ and R² are independently selected from (C₁-C₆)alkyl, (C₁-C₆)alkoxy, (C₂-C₆)alkenyl, (C₂-C₆)alkynyl, (C₃-C₆)cycloalkyl, (C₆-C₁₂)aryl, (C₆-C₁₂)aryloxy, (C₆-C₁₂)aryl(C₁-C₆)alkyl, heteroaryl, heteroaryl(C₁-C₆)alkyl, heterocyclic, (C₂-C₆)alkanoyl and (C₂-C₇)acyl, each of which may be unsubstituted or substituted with from 1 to 3 substituents selected from the group consisting of halogen or an isotope thereof, OH, (C₁-C₄)alkoxy, SH, NH₂, (C₁-C₄)alkylamino, di((C₁-C₄)alkyl)amino, carboxy, (C₁-C₄)alkoxycarbonyl, (C₂-C₄)alkanoyl, oxo, amido, CN, CNS, SCN, CNO, OCN and NHOH;

R³ and R⁴ are each independently hydrogen or a group selected from (C₁-C₄)alkyl, (C₂-C₄)alkenyl, (C₂-C₄)alkynyl, (C₃-C₆)cycloalkyl, (C₆-C₁₂)aryl, (C₆-C₁₂)aryl(C₁-C₄)alkyl, heteroaryl, heteroaryl(C₁-C₄)alkyl, heterocyclic, (C₁-C₄)alkoxycarbonyl and (C₂-C₅)acyl, each of which may be unsubstituted or substituted with from 1 to 3 substituents selected from the group consisting of halogen, OH, (C₁-C₄)alkoxy, SH, NH₂, (C₁-C₄)alkylamino, di((C₁-C₄)alkyl)amino, carboxy, (C₁-C₄)alkoxycarbonyl, (C₁-C₄)alkanoyl, oxo, amido, CN, CNS, SCN, CNO, OCN and NHOH,

or R³ and R⁴ together are (C₂-C₇)alkylidene which may be optionally substituted with from 1 to 3 substituents selected from the group consisting of halogen, OH, (C₁-C₄)alkoxy, SH, NH₂, (C₁-C₄)alkylamino, di((C₁-C₄)alkyl)amino, carboxy, (C₁-C₄)alkoxycarbonyl, (C₁-C₄)alkanoyl, oxo, amido, CN, CNS, SCN, CNO, OCN and NHOH;

m and n are independently 0, 1 or 2; and

p is 1;

wherein at each occurrence
(i) alkenyl has the meaning of ethylenically mono- or di-unsaturated alkyl or ethylenically mono- or di-unsaturated cycloalkyl;
(ii) cycloalkyl has the meaning of cyclic alkyl, or alkyl substituted cyclic alkyl;
(iii) heteroaryl has the meaning of single, polynuclear, conjugated and fused residues of aromatic heterocyclic ring systems.

Non-limiting examples of compounds of formula (I)

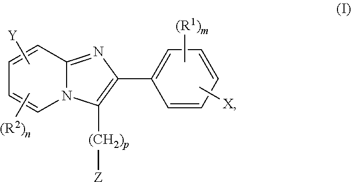

useful in the described methods include compounds wherein:

Y is selected from F, Cl, Br, I, CN and OH;

Z is selected from N(R³)C(O)R⁴ and C(O)NR³R⁴;

R¹ and R² are independently selected from (C₁-C₃)alkyl, (C₁-C₃)alkoxy, (C₂-C₃)alkenyl, (C₅-C₆)cycloalkyl, phenyl, naphthyl, phenoxy, naphthyloxy, benzyl, pyridyl, furanyl, thienyl, piperidinyl, morpholinyl, tetrahydrofuranyl, dioxanyl, (C₂-C₄)alkanoyl and (C₂-C₄)acyl, each of which may be unsubstituted or substituted with from a substituent selected from the group consisting of halogen, OH, (C₂-C₄)alkoxy, NH₂, (C₁-C₃)alkylamino, di((C₁-C₃)alkyl)amino, carboxy, (C₁-C₃)alkoxycarbonyl, (C₂-C₄)alkanoyl, oxo and amido;

R³ and R⁴ are each independently hydrogen or a group selected from (C₁-C₃)alkyl, (C₂-C₃)alkenyl, (C₅-C₆)cycloalkyl, phenyl, naphthyl, benzyl and (C₂-C₄)acyl, each of which may be unsubstituted or substituted with a substituent selected from the group consisting of halogen, OH, (C₁-C₃)alkoxy, NH₂, (C—C₃)alkylamino, di((C₁-C₃)alkyl)amino, carboxy, (C₁-C₃)alkoxycarbonyl, (C₂-C₄)alkanoyl, oxo and amido,

or R³ and R⁴ together are (C₂-C₃)alkylidene which may be optionally substituted with from a substituent selected from the group consisting of halogen, OH, (C₁-C₃)alkoxy, NH₂, (C₁-C₃)alkylamino, di((C₁-C₃)alkyl)amino, carboxy, (C₁-C₃)alkoxycarbonyl, (C₂-C₄)alkanoyl, oxo and amido; and

m and n are independently 0 or 1.

Non-limiting examples of compounds of formula (I) useful in the described methods, include compounds that are 2-(4′-iodophenyl)-imidazol[1,2-a]pyridine-3-acetamide derivatives of formula (IA)

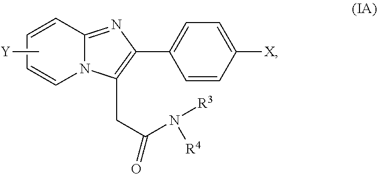

wherein:

X is iodine or an isotope thereof.

Y is halogen; and

R³ and R⁴ are independently selected from hydrogen, (C₁-C₄)alkyl and (C₂-C₄)alkenyl, or R³ and R⁴ taken together are (C₂-C₃)alkylidine.

A typical example of a compound of formula (IA) is [¹²⁵I]CLINDE, wherein: X is ¹²⁵I; Y is Cl; m and n are 0; and R³ and R⁴ CH₂CH₃.

Alternative, non-limiting examples of compounds of formula (I) useful in the described methods, include compounds that are derivatives of formula (IB)

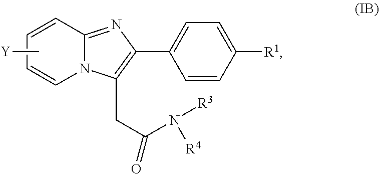

wherein:

Y is halogen:

R¹ is independently selected from (C₁-C₆)alkyl, (C₁-C₆)alkoxy, (C₂-C₆)alkenyl, (C₂-C₆)alkynyl, (C₃-C₅)cycloalkyl, (C₆-C₁₂)aryl, (C₆-C₁₂)aryloxy, (C₆-C₁₂)aryl(C₁-C₆)alkyl, heteroaryl, heteroaryl(C₁-C₆)alkyl, heterocyclic, (C₂-C₆)alkanoyl and (C₂-C₇)acyl, each of which may be unsubstituted or substituted with from 1 to 3 substituents selected from the group consisting of halogen or an isotope thereof, OH, (C₁-C₄)alkoxy, SH, NH₂, (C₁-C₄)alkylamino, di((C₁-C₄)alkyl)amino, carboxy, (C₁-C₄)alkoxycarbonyl, (C₂-C₄)alkanoyl, oxo, amido, CN, CNS, SCN, CNO, OCN and NHOH; and

R³ and R⁴ are independently selected from hydrogen, (C₁-C₄)alkyl and (C₂-C₄)alkenyl, or R³ and R⁴ taken together are (C₂-C₃)alkylidine.

A typical example of a compound of formula (IB) is [¹⁸F]PBR111, wherein: Y is Cl; R¹ is OCH₂CH₂¹⁸F; and R³ and R⁴ CH₂CH₃.

The methods and assays of screening and identification involve administration or exposure of the candidate compounds to the non-human animals, tissues, cells and/or immortalised cell lines of the present invention, and assessing the effect of these candidate compounds upon the phenotype of the non-human animals, tissues, cells and/or immortalised cell lines or the expression levels of TSPO and TSPO-associated gene products in the non-human animals, tissue, cells and immortalised cell lines. The methods may involve a comparison of phenotypes or expression levels of wild-type non-human animal, tissues, cells and/or immortalised cell lines of the same species to determine the nature of any effect the candidate compound may be having on the phenotype or expression levels in the test non-human animals, tissue, cells and immortalised cell lines of the present invention being used in the methods.

It would be understood that the phenotype of an animal comprises the animal's observable traits, which include but are not limited to, morphology, biochemical characteristics, physiology and behaviour. These traits can be observed in the animal, or in samples derived from an animal, such as tissues or cells, or from immortalised cell lines derived from an animal Biochemical characteristics may include, for example, the function and interactions of gene products of interest. The phenotype of an animal can be influenced by both genetics factors and external environmental factors, such as the administration of, or exposure to, a candidate compound. Expression levels of any gene product can be measured in absolute terms or in respect to control gene products. The control gene product may be the same gene product or a different gene product.

Methods of screening and identifying candidate compounds using the non-human animals, cells, tissues and immortalised cell lines of the present invention may provide different pieces of information, including but not limited to the following:

Firstly, the methods may reveal the amount of non-selective or non-specific binding or interaction (i.e. amount of compound that binds targets other than TSPO) of a candidate compound in animals comprising cells with at least one copy of a non-functional TSPO gene. Secondly, an animal in which a functional TSPO gene product is not present or expressed at a lower level than a wild-type animal may reveal biological pathways and mechanisms by which the animal has compensated for the absent or lower expression of a functional TSPO gene product, such as the absence, decreased or increased expression of TSPO-associated gene products, which in turn influences the regulation of other associated or functionally linked genes. Thirdly, heterozygous animals in which only one copy of the TSPO gene is non-functional may show adaptive or compensatory regulation in other associated or functionally linked genes, which may vary from the adaptive or compensatory response seen animals in which both copies of the TSPO gene is non-functional.

It would be understood that the selectivity and specificity of known or new candidate compounds with respect to the binding or functional modulation to TSPO or TSPO-associated gene products in a subject requires testing of these compounds on non-human animals, tissues, cells or immortalised cell lines that have modified expression of endogenous TSPO gene products, such as the non-human animals, tissues, cells or immortalised cell lines of the present invention as described herein.

Accordingly, the present invention also provides methods for screening the binding specificity or selectivity of a candidate compound to TSPO or TSPO-associated gene products. The method comprises the steps of administering or exposing a candidate compound to a non-human animal, tissue, cell or immortalised cell line of the present invention as herein described and comparing the binding selectivity or specificity of the candidate compound to TSPO or TSPO-associated gene products in the test sample with that of a wild-type sample.

Amongst others, and with reference to the Examples below, namely Example 13, the inventors have shown that TSPO plays a role in regulating the systemic and/or cellular energy household, in regulating mitochondrial oxidative pathways, mitochondrial ATP production, and energy storage in response to high-fat diet. Specifically, the inventors have shown that increased energy intake in the form of a prolonged, high fat diet leads to a significantly reduced (and less than expected) weight gain in TSPO knock-out animals according to the present invention when compared to wild-type animals. Accordingly, a role for TSPO and TSPO-mediated signalling in the protection against obesity resulting from a high fat diet has been provided.

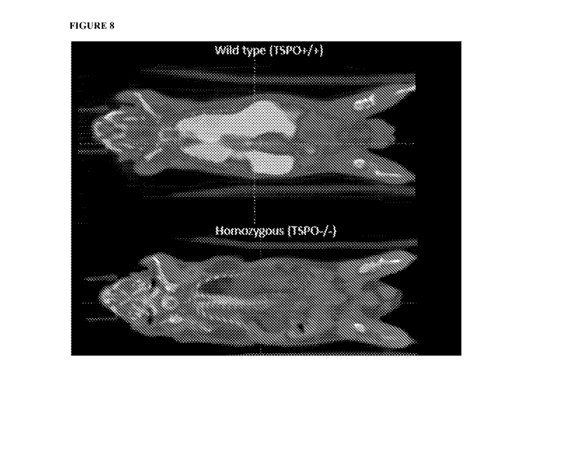

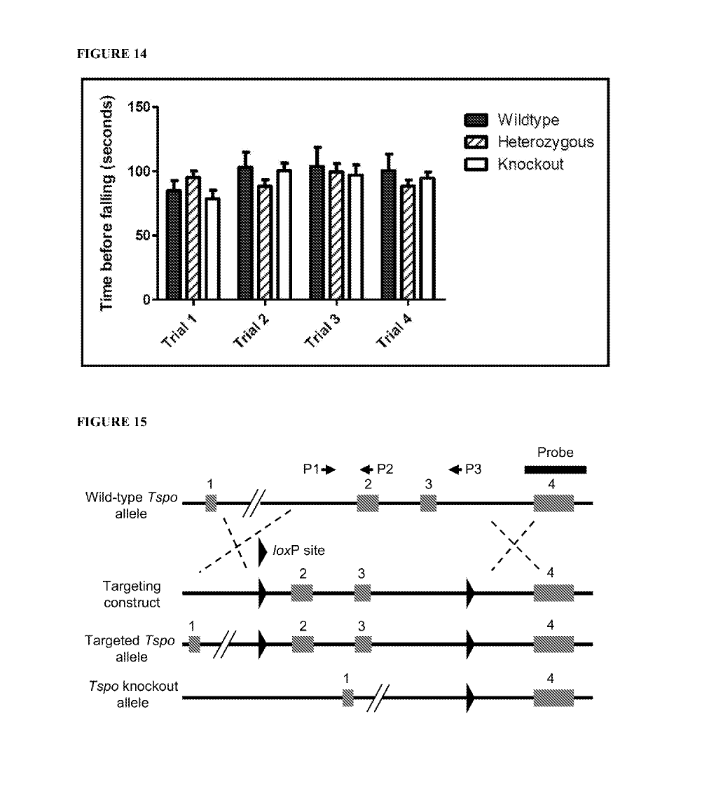

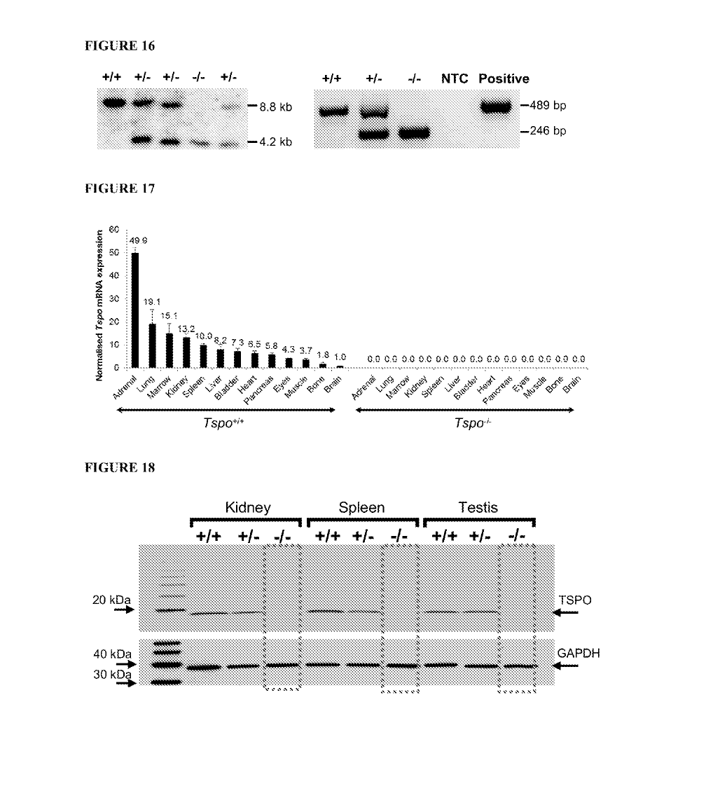

Referring to the Examples below, and specifically to Example 5 (FIG. 9), as well as Example 8, FIGS. 15 to 21), the inventors have further shown that in accordance with methods of the present invention, compounds of general formula (I) have been identified as being highly specific and selective TSPO binding molecules. As is well established in the field, specific and selective receptor binding molecules are potent inhibitors of receptor function and are often used to generate “pharmacological knock-outs”, i.e. to inhibit receptor function so efficiently that a phenotype mirroring the global loss of function achieved by a genetic knock-out can be generated.

Accordingly, in certain non-limiting embodiments, the present invention relates the use of a compound of general formula (I)

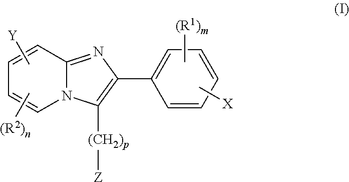

to inhibit TSPO function, wherein

X is absent, iodine or an isotope thereof;

Y is selected from F, Cl, Br, I, OH, SH, NH₂, CN and COOH;

Z is selected from N(R³)C(O)R⁴ and C(O)NR¹R⁴;

R¹ and R² are independently selected from (C₁-C₆)alkyl, (C₁-C₆)alkoxy, (C₂-C₆)alkenyl, (C₂-C₆)alkynyl, (C₃-C₆)cycloalkyl, (C₆-C₁₂)aryl, (C₆-C₁₂)aryloxy, (C₆-C₁₂)aryl(C₁-C₆)alkyl, heteroaryl, heteroaryl(C₁-C₆)alkyl, heterocyclic, (C₂-C₆)alkanoyl and (C₂-C₇)acyl, each of which may be unsubstituted or substituted with from 1 to 3 substituents selected from the group consisting of halogen or an isotope thereof, OH, (C₁-C₄)alkoxy, SH, NH₂, (C₁-C₄)alkylamino, di((C₁-C₄)alkyl)amino, carboxy, (C₁-C₄)alkoxycarbonyl, (C₂-C₄)alkanoyl, oxo, amido, CN, CNS, SCN, CNO, OCN and NHOH;

R³ and R⁴ are each independently hydrogen or a group selected from (C₁-C₄)alkyl, (C₂-C₄)alkenyl, (C₂-C₄)alkynyl, (C₃-C₆)cycloalkyl, (C₆-C₁₂)aryl, (C₆-C₁₂)aryl(C₁-C₄)alkyl, heteroaryl, heteroaryl(C₁-C₄)alkyl, heterocyclic, (C₁-C₄)alkoxycarbonyl and (C₂-C₅)acyl, each of which may be unsubstituted or substituted with from 1 to 3 substituents selected from the group consisting of halogen, OH, (C₁-C₄)alkoxy, SH, NH₂, (C₁-C₄)alkylamino, di((C₁-C₄)alkyl)amino, carboxy, (C₁-C₄)alkoxycarbonyl, (C₁-C₄)alkanoyl, oxo. amido, CN. CNS, SCN, CNO, OCN and NHOH,

or R³ and R⁴ together are (C₂-C₇)alkylidene which may be optionally substituted with from 1 to 3 substituents selected from the group consisting of halogen. OH, (C₁-C₄)alkoxy, SH, NH₂, (C₁-C₄)alkylamino, di((C₁-C₄)alkyl)amino, carboxy, (C₁-C₄)alkoxycarbonyl, (C₁-C₄)alkanoyl, oxo, amido, CN, CNS, SCN, CNO, OCN and NHOH;

m and n are independently 0, 1 or 2; and

p is 1;

wherein at each occurrence
(i) alkenyl has the meaning of ethylenically mono- or di-unsaturated alkyl or ethylenically mono- or di-unsaturated cycloalkyl;
(ii) cycloalkyl has the meaning of cyclic alkyl, or alkyl substituted cyclic alkyl;
(iii) heteroaryl has the meaning of single, polynuclear, conjugated and fused residues of aromatic heterocyclic ring systems.

In other non-limiting embodiments, the present invention relates to the use of a compound of general formula (I)

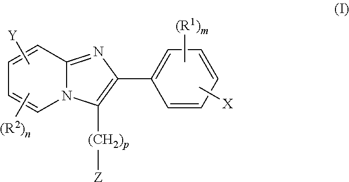

to alter the systemic and/or cellular energy household, wherein

X is absent, iodine or an isotope thereof:

Y is selected from F, Cl, Br, I, OH, SH, NH₂, CN and COOH;

Z is selected from N(R³)C(O)R⁴ and C(O)NR³R⁴;

R¹ and R² are independently selected from (C₁-C₆)alkyl, (C₁-C₆)alkoxy, (C₂-C₆)alkenyl, (C₂-C₆)alkynyl, (C₃-C₆)cycloalkyl, (C₆-C₁₂)aryl, (C₆-C₁₂)aryloxy, (C₆-C₁₂)aryl(C₁-C₆)alkyl, heteroaryl, heteroaryl(C₁-C₆)alkyl, heterocyclic, (C₂-C₆)alkanoyl and (C₂-C₇)acyl, each of which may be unsubstituted or substituted with from 1 to 3 substituents selected from the group consisting of halogen or an isotope thereof, OH, (C₁-C₄)alkoxy, SH, NH₂, (C₁-C₄)alkylamino, di((C₁-C₄)alkyl)amino, carboxy, (C₁-C₄)alkoxycarbonyl, (C₂-C₄)alkanoyl, oxo, amido, CN, CNS, SCN, CNO, OCN and NHOH:

R³ and R⁴ are each independently hydrogen or a group selected from (C₁-C₄)alkyl, (C₂-C₄)alkenyl, (C₂-C₄)alkynyl, (C₃-C₆)cycloalkyl, (C₆-C₁₂)aryl, (C₆-C₁₂)aryl(C₁-C₄)alkyl, heteroaryl, heteroaryl(C₁-C₄)alkyl, heterocyclic, (C₁-C₄)alkoxycarbonyl and (C₂-C₅)acyl, each of which may be unsubstituted or substituted with from 1 to 3 substituents selected from the group consisting of halogen, OH, (C₁-C₄)alkoxy, SH, NH₂, (C₁-C₄)alkylamino, di((C₁-C₄)alkyl)amino, carboxy, (C₁-C₄)alkoxycarbonyl, (C₁-C₄)alkanoyl, oxo, amido, CN, CNS, SCN, CNO, OCN and NHOH,

or R³ and R⁴ together are (C₂-C₇)alkylidene which may be optionally substituted with from 1 to 3 substituents selected from the group consisting of halogen, OH, (C₁-C₄)alkoxy, SH, NH₂, (C₁-C₄)alkylamino, di((C₁-C₄)alkyl)amino, carboxy, (C₁-C₄)alkoxycarbonyl, (C₁-C₄)alkanoyl, oxo, amido, CN, CNS, SCN. CNO. OCN and NHOH;

m and n are independently 0, 1 or 2; and

p is 1;

wherein at each occurrence
(i) alkenyl has the meaning of ethylenically mono- or di-unsaturated alkyl or ethylenically mono- or di-unsaturated cycloalkyl;
(ii) cycloalkyl has the meaning of cyclic alkyl, or alkyl substituted cyclic alkyl;

(iii) heteroaryl has the meaning of single, polynuclear, conjugated and fused residues of aromatic heterocyclic ring systems.

In other non-limiting embodiments, the present invention relates to the use of a compound of general formula (I)

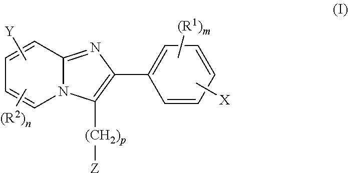

to alter a mitochondrial oxidative pathway, wherein

X is absent, iodine or an isotope thereof;

Y is selected from F, Cl, Br, I, OH, SH, NH₂, CN and COOH;

Z is selected from N(R³)C(O)R⁴ and C(O)NR³R⁴:

R¹ and R² are independently selected from (C₁-C₆)alkyl, (C₁-C₆)alkoxy, (C₂-C₆)alkenyl, (C₂-C₆)alkynyl, (C₃-C₆)cycloalkyl, (C₆-C₁₂)aryl, (C₆-C₁₂)aryloxy, (C₆-C₁₂)aryl(C₁-C₆)alkyl, heteroaryl, heteroaryl(C₁-C₆)alkyl, heterocyclic, (C₂-C₆)alkanoyl and (C₂-C₇)acyl, each of which may be unsubstituted or substituted with from 1 to 3 substituents selected from the group consisting of halogen or an isotope thereof, OH, (C₁-C₄)alkoxy, SH, NH₂, (C₁-C₄)alkylamino, di((C₁-C₄)alkyl)amino, carboxy, (C₁-C₄)alkoxycarbonyl, (C₂-C₄)alkanoyl, oxo, amido, CN, CNS, SCN, CNO, OCN and NHOH:

R³ and R⁴ are each independently hydrogen or a group selected from (C₁-C₄)alkyl, (C₂-C₄)alkenyl, (C₂-C₄)alkynyl, (C₃-C₆)cycloalkyl, (C₆-C₁₂)aryl, (C₆-C₁₂)aryl(C₁-C₄)alkyl, heteroaryl, heteroaryl(C₁-C₄)alkyl, heterocyclic, (C₁-C₄)alkoxycarbonyl and (C₂-C₅)acyl, each of which may be unsubstituted or substituted with from 1 to 3 substituents selected from the group consisting of halogen. OH, (C₁-C₄)alkoxy, SH, NH₂, (C₁-C₄)alkylamino, di((C₁-C₄)alkyl)amino, carboxy, (C₁-C₄)alkoxycarbonyl, (C₁-C₄)alkanoyl, oxo, amido, CN, CNS, SCN, CNO, OCN and NHOH,

or R³ and R⁴ together are (C₂-C₇)alkylidene which may be optionally substituted with from 1 to 3 substituents selected from the group consisting of halogen, OH, (C₁-C₄)alkoxy, SH, NH₂, (C₁-C₄)alkylamino, di((C₁-C₄)alkyl)amino, carboxy, (C₁-C₄)alkoxycarbonyl, (C₁-C₄)alkanoyl, oxo, amido, CN, CNS, SCN, CNO, OCN and NHOH;

m and n are independently 0, 1 or 2; and

p is 1;

wherein at each occurrence
(i) alkenyl has the meaning of ethylenically mono- or di-unsaturated alkyl or ethylenically mono- or di-unsaturated cycloalkyl;
(ii) cycloalkyl has the meaning of cyclic alkyl, or alkyl substituted cyclic alkyl;
(iii) heteroaryl has the meaning of single, polynuclear, conjugated and fused residues of aromatic heterocyclic ring systems.

In further non-limiting embodiments, the present invention relates to the use of a compound of general formula (I)

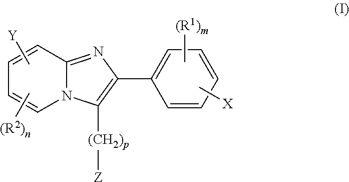

to regulate mitochondrial ATP production, wherein

X is absent, iodine or an isotope thereof;

Y is selected from F, Cl, Br, I, OH, SH, NH₂, CN and COOH;

Z is selected from N(R³)C(O)R⁴ and C(O)NR³R⁴;

R¹ and R² are independently selected from (C₁-C₆)alkyl, (C₁-C₆)alkoxy, (C₂-C₆)alkenyl, (C₂-C₆)alkynyl, (C₃-C₆)cycloalkyl, (C₆-C₁₂)aryl, (C₆-C₁₂)aryloxy, (C₆-C₁₂)aryl(C₁-C₆)alkyl, heteroaryl, heteroaryl(C₁-C₆)alkyl, heterocyclic, (C₂-C₆)alkanoyl and (C₂-C₇)acyl, each of which may be unsubstituted or substituted with from 1 to 3 substituents selected from the group consisting of halogen or an isotope thereof, OH, (C₁-C₄)alkoxy, SH, NH₂, (C₁-C₄)alkylamino, di((C₁-C₄)alkyl)amino, carboxy, (C₁-C₄)alkoxycarbonyl, (C₂-C₄)alkanoyl, oxo, amido, CN, CNS, SCN, CNO, OCN and NHOH;

R³ and R⁴ are each independently hydrogen or a group selected from (C₁-C₄)alkyl, (C₂-C₄)alkenyl, (C₂-C₄)alkynyl, (C₃-C₆)cycloalkyl, (C₆-C₁₂)aryl, (C₆-C₁₂)aryl(C₁-C₄)alkyl, heteroaryl, heteroaryl(C₁-C₄)alkyl, heterocyclic, (C₁-C₄)alkoxycarbonyl and (C₂-C₅)acyl, each of which may be unsubstituted or substituted with from 1 to 3 substituents selected from the group consisting of halogen, OH, (C₁-C₄)alkoxy, SH, NH₂, (C₁-C₄)alkylamino, di((C₁-C₄)alkyl)amino, carboxy, (C₁-C₄)alkoxycarbonyl, (C₁-C₄)alkanoyl, oxo, amido, CN, CNS, SCN, CNO, OCN and NHOH,

or R³ and R⁴ together are (C₂-C₇)alkylidene which may be optionally substituted with from 1 to 3 substituents selected from the group consisting of halogen, OH, (C₁-C₄)alkoxy, SH, NH₂, (C₁-C₄)alkylamino, di((C₁-C₄)alkyl)amino, carboxy, (C₁-C₄)alkoxycarbonyl, (C₁-C₄)alkanoyl, oxo, amido, CN, CNS, SCN, CNO, OCN and NHOH;

m and n are independently 0, 1 or 2; and

p is 1;

wherein at each occurrence
(i) alkenyl has the meaning of ethylenically mono- or di-unsaturated alkyl or ethylenically mono- or di-unsaturated cycloalkyl;
(ii) cycloalkyl has the meaning of cyclic alkyl, or alkyl substituted cyclic alkyl;
(iii) heteroaryl has the meaning of single, polynuclear, conjugated and fused residues of aromatic heterocyclic ring systems.

In further non-limiting embodiments, the present invention relates to the use of a compound of general formula (I)

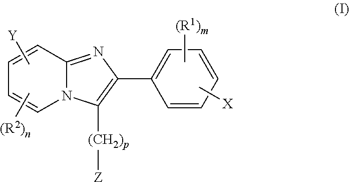

to regulate TSPO-mediated signalling, wherein

X is absent, iodine or an isotope thereof;

Y is selected from F, Cl, Br, I, OH, SH, NH₂, CN and COOH;

Z is selected from N(R³)C(O)R⁴ and C(O)NR³R⁴;

R¹ and R² are independently selected from (C₁-C₆)alkyl, (C₁-C₆)alkoxy, (C₂-C₆)alkenyl, (C₂-C₆)alkynyl, (C₃-C₆)cycloalkyl, (C₆-C₁₂)aryl, (C₆-C₁₂)aryloxy, (C₆-C₁₂)aryl(C₁-C₆)alkyl, heteroaryl, heteroaryl(C₁-C₆)alkyl, heterocyclic, (C₂-C₆)alkanoyl and (C₂-C₇)acyl, each of which may be unsubstituted or substituted with from 1 to 3 substituents selected from the group consisting of halogen or an isotope thereof, OH, (C₁-C₄)alkoxy, SH, NH₂, (C₁-C₄)alkylamino, di((C₁-C₄)alkyl)amino, carboxy, (C₁-C₄)alkoxycarbonyl, (C₂-C₄)alkanoyl, oxo, amido, CN, CNS, SCN, CNO, OCN and NHOH;

R³ and R⁴ are each independently hydrogen or a group selected from (C₁-C₄)alkyl, (C₂-C₄)alkenyl, (C₂-C₄)alkynyl, (C₃-C₆)cycloalkyl, (C₆-C₁₂)aryl, (C₆-C₁₂)aryl(C₁-C₄)alkyl, heteroaryl, heteroaryl(C₁-C₄)alkyl, heterocyclic, (C₁-C₄)alkoxycarbonyl and (C₂-C₅)acyl, each of which may be unsubstituted or substituted with from 1 to 3 substituents selected from the group consisting of halogen, OH, (C₁-C₄)alkoxy, SH, NH₂, (C₁-C₄)alkylamino, di((C₁-C₄)alkyl)amino, carboxy, (C₁-C₄)alkoxycarbonyl, (C₁-C₄)alkanoyl, oxo, amido, CN, CNS, SCN, CNO, OCN and NHOH,

or R³ and R⁴ together are (C₂-C₇)alkylidene which may be optionally substituted with from 1 to 3 substituents selected from the group consisting of halogen, OH, (C₁-C₄)alkoxy, SH, NH₂, (C₁-C₄)alkylamino, di((C₁-C₄)alkyl)amino, carboxy, (C₁-C₄)alkoxycarbonyl, (C₁-C₄)alkanoyl, oxo, amido, CN, CNS, SCN, CNO, OCN and NHOH;

m and n are independently 0, 1 or 2; and

p is 1:

wherein at each occurrence
(i) alkenyl has the meaning of ethylenically mono- or di-unsaturated alkyl or ethylenically mono- or di-unsaturated cycloalkyl;
(ii) cycloalkyl has the meaning of cyclic alkyl, or alkyl substituted cyclic alkyl;
(iii) heteroaryl has the meaning of single, polynuclear, conjugated and fused residues of aromatic heterocyclic ring systems.

In further non-limiting embodiments, the present invention relates to the use of a compound of general formula (I)

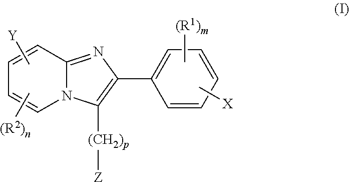

to regulate TSPO-mediated energy storage, wherein

X is absent, iodine or an isotope thereof;

Y is selected from F, Cl, Br, I, OH, SH, NH₂, CN and COOH;

Z is selected from N(R³)C(O)R⁴ and C(O)NR³R⁴;

R¹ and R² are independently selected from (C₁-C₆)alkyl, (C₁-C₆)alkoxy, (C₂-C₆)alkenyl, (C₂-C₆)alkynyl, (C₃-C₆)cycloalkyl, (C₆-C₁₂)aryl, (C₆-C₁₂)aryloxy, (C₆-C₁₂)aryl(C₁-C₆)alkyl, heteroaryl, heteroaryl(C₁-C₆)alkyl, heterocyclic, (C₂-C₄)alkanoyl and (C₂-C₇)acyl, each of which may be unsubstituted or substituted with from 1 to 3 substituents selected from the group consisting of halogen or an isotope thereof, OH, (C₁-C₄)alkoxy, SH, NH₂, (C₁-C₄)alkylamino, di((C₁-C₄)alkyl)amino, carboxy, (C₁-C₄)alkoxycarbonyl, (C₂-C₄)alkanoyl, oxo, amido, CN, CNS, SCN, CNO, OCN and NHOH:

R³ and R⁴ are each independently hydrogen or a group selected from (C₁-C₄)alkyl, (C₂-C₄)alkenyl, (C₂-C₄)alkynyl, (C₃-C₆)cycloalkyl, (C₆-C₁₂)aryl, (C₆-C₁₂)aryl(C₁-C₄)alkyl, heteroaryl, heteroaryl(C₁-C₄)alkyl, heterocyclic, (C₁-C₄)alkoxycarbonyl and (C₂-C₅)acyl, each of which may be unsubstituted or substituted with from 1 to 3 substituents selected from the group consisting of halogen, OH, (C₁-C₄)alkoxy, SH, NH₂, (C₁-C₄)alkylamino, di((C₁-C₄)alkyl)amino, carboxy, (C₁-C₄)alkoxycarbonyl, (C₁-C₄)alkanoyl, oxo, amido, CN, CNS, SCN, CNO, OCN and NHOH,

or R³ and R⁴ together are (C₂-C₇)alkylidene which may be optionally substituted with from 1 to 3 substituents selected from the group consisting of halogen, OH, (C₁-C₄)alkoxy, SH, NH₂, (C₁-C₄)alkylamino, di((C₁-C₄)alkyl)amino, carboxy, (C₁-C₄)alkoxycarbonyl, (C₁-C₄)alkanoyl, oxo, amido, CN, CNS, SCN, CNO, OCN and NHOH;

m and n are independently 0, 1 or 2; and

p is 1;

wherein at each occurrence
(i) alkenyl has the meaning of ethylenically mono- or di-unsaturated alkyl or ethylenically mono- or di-unsaturated cycloalkyl;
(ii) cycloalkyl has the meaning of cyclic alkyl, or alkyl substituted cyclic alkyl;
(iii) heteroaryl has the meaning of single, polynuclear, conjugated and fused residues of aromatic heterocyclic ring systems.

In some embodiments the above-described uses of the present invention provide protection against obesity. In other embodiments the above-described uses of the present invention provide protection against high fat diet-induced weight gain.

Non-limiting examples of compounds of formula (I) used in accordance with the present invention include PBR111 and CLINDE.

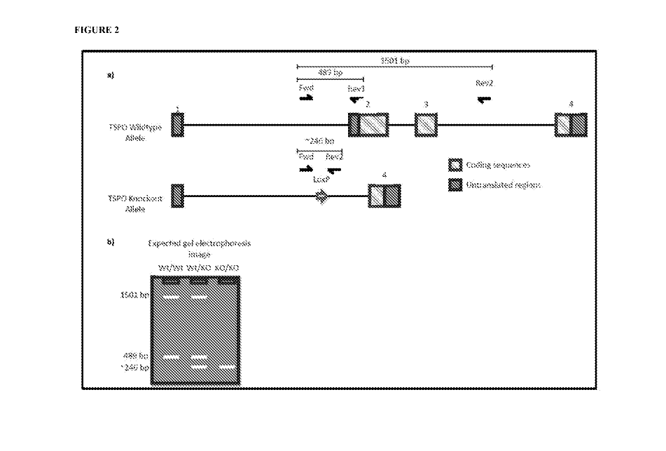

As indicated above, and with reference to Example 8 and FIG. 2, the TSPO knock-out mice illustrate the surprising finding that global loss of TSPO function appears to have no or only a minimal effect on the activation of microglia after neuronal injury.

Accordingly, in a further, non-limiting embodiment, the present invention relates to the use of a compound of general formula (I)

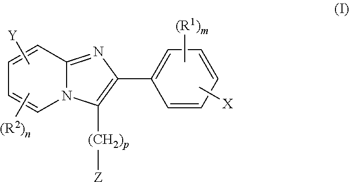

to investigate inflammatory responses associated with neuronal injury, wherein

X is absent, iodine or an isotope thereof;

Y is selected from F, Cl, Br, I, OH, SH, NH₂, CN and COOH;

Z is selected from N(R³)C(O)R⁴ and C(O)NR³R⁴;

R¹ and R² are independently selected from (C₁-C₆)alkyl, (C₁-C₆)alkoxy, (C₂-C₆)alkenyl, (C₂-C₆)alkynyl, (C₃-C₆)cycloalkyl, (C₆-C₁₂)aryl, (C₆-C₁₂)aryloxy, (C₆-C₂)aryl(C₁-C₆)alkyl, heteroaryl, heteroaryl(C₁-C₆)alkyl, heterocyclic, (C₂-C₆)alkanoyl and (C₂-C₇)acyl, each of which may be unsubstituted or substituted with from 1 to 3 substituents selected from the group consisting of halogen or an isotope thereof, OH, (C₁-C₄)alkoxy, SH, NH₂, (C₁-C₄)alkylamino, di((C₁-C₄)alkyl)amino, carboxy, (C₁-C₄)alkoxycarbonyl, (C₂-C₄)alkanoyl, oxo, amido, CN, CNS, SCN, CNO, OCN and NHOH;

R³ and R⁴ are each independently hydrogen or a group selected from (C₁-C₄)alkyl, (C₂-C₄)alkenyl, (C₂-C₄)alkynyl, (C₃-C₆)cycloalkyl, (C₆-C₁₂)aryl, (C₆-C₁₂)aryl(C₁-C₄)alkyl, heteroaryl, heteroaryl(C₁-C₄)alkyl, heterocyclic, (C₁-C₄)alkoxycarbonyl and (C₂-C₅)acyl, each of which may be unsubstituted or substituted with from 1 to 3 substituents selected from the group consisting of halogen, OH, (C₁-C₄)alkoxy, SH, NH₂, (C₁-C₄)alkylamino, di((C₁-C₄)alkyl)amino, carboxy, (C₁-C₄)alkoxycarbonyl, (C₁-C₄)alkanoyl, oxo, amido, CN, CNS, SCN, CNO, OCN and NHOH,

or R³ and R⁴ together are (C₂-C₇)alkylidene which may be optionally substituted with from 1 to 3 substituents selected from the group consisting of halogen, OH, (C₁-C₄)alkoxy, SH, NH₂, (C₁-C₄)alkylamino, di((C₁-C₄)alkyl)amino, carboxy, (C₁-C₄)alkoxycarbonyl, (C₁-C₄)alkanoyl, oxo, amido, CN, CNS, SCN, CNO, OCN and NHOH;

m and n are independently 0, 1 or 2; and

p is 1;

wherein at each occurrence
(i) alkenyl has the meaning of ethylenically mono- or di-unsaturated alkyl or ethylenically mono- or di-unsaturated cycloalkyl;
(ii) cycloalkyl has the meaning of cyclic alkyl, or alkyl substituted cyclic alkyl;
(iii) heteroaryl has the meaning of single, polynuclear, conjugated and fused residues of aromatic heterocyclic ring systems.

Subjects

The subject may be human or may be a non-human. Reference to a subject or individual means a human or a non-human, such as an individual of any species of social, economic or research importance including but not limited to members of the classifications of ovine, bovine, equine, porcine, feline, canine, primates, rodents, especially domesticated members of those classifications, such as sheep, cattle, horses and dogs.

It will be appreciated by persons of ordinary skill in the art that numerous variations and/or modifications can be made to the present invention as disclosed in the specific embodiments without departing from the spirit or scope of the present invention as broadly described. The present embodiments are, therefore, to be considered in all respects as illustrative and not restrictive.

### EXAMPLES

### Example 1

### Development of a TSPO Knock-Out Animal

Materials and Methods

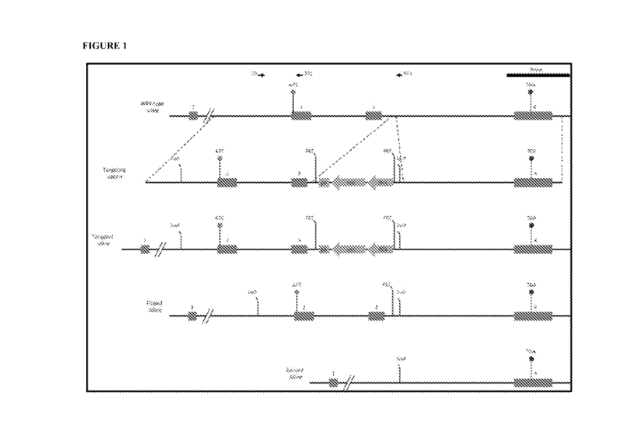

Construct Design and Transgenic Animal Generation Development of the TSPO knock-out mice was performed by Ozgene (Bentley DC, WA, Australia). TSPO knock-out mice were created using a Cre-Lox recombination method. To generate TSPO knock-out mice, a targeting construct homologous to the wild-type TSPO allele with several additions was created (See FIG. 1). The construct also contained a pair of LoxP sites flanking exons 2 and 3 which include the TSPO start codon, a neomycin cassette to screen for successful acquisition of the targeting construct and also Flippase recognition target (FRT) sites. The FRT sites were included to enable the production of conditional knock-out mice at a later stage. The neomycin cassette confers neomycin resistance to cells which successfully incorporate the construct. The construct was delivered into Bruce4 mouse embryonic stem (ES) cells through electroporation, thereby allowing the modified TSPO sequence from the targeting construct to replace the wild-type TSPO sequence through homologous recombination. G418, an antibiotic which blocks polypeptide synthesis in eukaryotic cells, resistance to which is conferred by the neomycin gene, was subsequently used to screen for ES cells containing the targeting construct.

Selected ES cells were injected into an albino C57BL/6 blastocyst and implanted into a foster mother giving rise to chimeric offspring. Chimeric mice were crossed with wild-type albino C57BL/6 mice to produce offspring heterozygous for the modified TSPO sequence. Any offspring with black coats will contain the targeting construct as only albino blastocysts and breeding mates were used. Black mice heterozygous for the modified TSPO sequence were then crossed with C57BL/6 Cre deleter mice and offspring which were heterozygous for the TSPO gene were selected. These mice, heterozygous for the TSPO and Cre recombinase gene, were further crossed with wild-type C57BL/6 mice to remove the Cre recombinase gene. Offspring heterozygous for the TSPO gene and not expressing the Cre recombinase gene were then crossed with each other to produce TSPO homozygous knock-out, TSPO heterozygous knock-out and TSPO wild-type animals.

All animal procedures were approved by the University of Sydney Animal Ethics Committee and the ANSTO Animal Care and Ethics Committee. The TSPO homozygous knock-out mouse strain has been given the additional designation GuwiyangWurra (‘fire mouse’ in the local Dharawal language). Accordingly, reference to the TSPO homozygous knock-out mouse of the present invention may also be made by referring to the C57BL/6-TSPO^{tm1GuMu(GuwiyangWurra)} mouse or mouse strain.

Animal Breeding

TSPO knock-out animals were bred such that a suitable number of TSPO homozygous knock-out, TSPO heterozygous knock-out, and TSPO wild-type animals were available. To assess the fertility of the TSPO knock-out mice, TSPO homozygous knock-out mice were bred with both TSPO homozygous and heterozygous knock-out mice of both sexes.

Following birth, the genotype of each animal was then determined by stool gDNA extraction, PCR and gel electrophoresis. Animals from this generation were used for behavioural assessment; animal not used in behavioural assessment were used to establish a breeding colony and progeny from this colony were used to generate the weight and general health data.

Genotyping

Tissue Genomic DNA Isolation

Mouse tissue sample from distal tail region were collected after euthanasia. Approximately 1 cm of the distal tail was clipped and used for gDNA extraction. gDNA was collected from the tissue samples using a PureLink Genomic DNA Mini Kit (Qiagen) following the manufactures instructions. Briefly, the tail clip was placed into a sterile eppendorf tube to which 180 μL of PureLink Genomic Digestion Buffer and 20 μL Proteinase K was added. The tube was briefly vortexed and incubated at 55° C. with occasional vortexing until lysis was complete, typically this took approximately 3-4 hours. Following complete lysis, the lysate was centrifuged for 3 minutes at room temperature at 10000 g. The supernatant was transferred to a new eppendorf tube to which 20 μL of RNase A was added. This was then mixed by brief vortexing, and allowed to incubate at room temperature for 2 minutes.

Following incubation, 200 μL of PureLink Genomic Lysis/Binding Buffer and 200 μL of 96% ethanol was then added to the lysate and vortexed for 5 seconds. The entire lysate was centrifuged in 200 μL amounts through the PureLink Spin Column at 10000 g for 1 minute at room temperature. The spin columns were then washed with 500 μL of Wash Buffer 1 and 500 μL of Wash Buffer 2, centrifuging at room temperature at 10000 g for 1 minute and 3 minutes respectively. Following each wash the collection tube was discarded and a new one was used. To elude the gDNA, 100 μL of PureLink Genomic Elution Buffer was added to the spin columns, the spin column were placed in a sterile microcentrifuge tube and following 1 minute incubation at room temperature, centrifuged at room temperature at 10000 g. Eluted gDNA was stored at −20° C. until required.

Concentration of the gDNA was spectrophotometrically determined using a Nanodrop 2000c spectrophotometer.

Southern Blot Analysis Using Genomic DNA

Animals were genotyped by Southern blot analysis using genomic DNA isolated from mouse tail biopsies, digesting the DNA with ScaI and hybridising with a probe to generate an 8.8 kb fragment for the wild-type allele and a 4.2 kb fragment for the knock-out allele.

Stool Sample Genomic DNA Isolation

Stool samples were collected by placing a single mouse in a clean animal housing box, typically stool samples were obtained within 30 seconds. Stool samples were either used fresh or were snap frozen in liquid nitrogen and stored in −80° C. until required. gDNA was collected from the stool samples using a QIAamp DNA Stool Mini Kit (Qiagen) following the manufactures instructions. In brief, stool samples were homogenized through vortexing and triturating using a 1 mL pipette in 1.6 ml of ASL buffer. Samples were then centrifuged at room temperature at 10000 g for 1 minute and supernatant transferred into a new tube to which an InhibitEX tablet was added. The InhibitEX tablet was vortexed until dissolved and was allowed to incubate at room temperature for at least 1 minute. Following incubation, samples were centrifuged at room temperature at 10000 g for 3 minutes and the supernatant of each sample transferred to a new tube and centrifuged again at full speed for another 3 minutes. Following centrifugation, 600 μL of the supernatant was pipette into a new tube containing 25 μL of proteinase K and 600 μL of buffer AL, and this was combined through a 15 second vortex. The mixture was then incubated at 70° C. for 10 minutes.

To precipitate the DNA, 600 μL of ethanol was added to the mixture and was mixed by brief vortexing. The gDNA was collected by a spin column by centrifugation at room temperature at 10000 g for 1 minute and washed with 500 μL of buffer AW1 and 500 μL of buffer AW2, centrifuging at room temperature at 10000 g for 1 minute and 3 minutes respectively. To elude the gDNA, buffer AE was transferred directly onto the spin column membrane, incubated for at least 1 minute and centrifuged at full speed for 1 minute. The volume of buffer AE used varied depending on the size of the initial stool sample. Eluted gDNA was stored at −20° C. until required. Concentration of the gDNA was spectrophotometrically determined using a Nanodrop 2000c spectrophotometer. PCR was then performed on the isolated gDNA to determine the presence of the TSPO gene.

Polymerase Chain Reaction

For routine genotyping, PCR was performed on isolated gDNA using a set of three primers designed to differentiate between the different genotypes of the TSPO knock-out mice. The primer set consists of one forward (FP) and two reverse primers (RP1 and RP2) (See FIG. 1), with the wild-type and knock-out alleles respectively producing 2 and 1 unique PCR product sizes. This enabled a simple and efficient means of determining animal genotype upon standard gel electrophoresis and viewing under UV light. The expected PCR product band sizes for the wild-type allele are 489 and 1501 base pairs and for the knock-out allele approximately 246 base pairs. FIG. 2 shows a schematic representation of primer design and expected gel image). The principle of this design avoided non-detection as an indication of a knock-out allele, which conferred the advantage of not confusing the presence of the TSPO knock-out allele with poor or incorrect performance during the DNA extraction or PCR setup and allows for an overall higher accuracy during genotyping. See Table 1 for primers used during genotyping.

TABLE 1
Sequence of primers used in animal genotyping

| | Primer sequence |
|---|---|
| Forward primer | 5′-GGTAGACTAGTGTGGGAAGATTTGA-3′ |
| (FP) | |
| Reverse primer 1 | 5′-TAGATACTGACCCTATCTGGGATGT-3′ |
| (RP1) | |
| Reverse primer 2 | 5′-ATGGTGATTGCAACTGATGTTC-3′ |
| (RP2) | |

The PCR was performed in a final volume of 25 μL and consisted of 5 μL of Green GoTaq Reaction Buffer (Promega), 1.5 μL of MgCL2 (Promega), 0.5 μL of PCR Nucleotide Mix (Promega), 0.625 μL of forward primer, 0.3125 μL of reverse 1 primer, 0.625 μL of reverse 2 primer, 0.125 μL of GoTaq® Hot Start Polymerase (Promega) and approximately 200 ng of gDNA diluted with nuclease free H₂O to 16.3125 μL. PCR was performed using the T100 Thermal Cycler (Bio-Rad, Hercules, Calif., USA). PCR amplification run cycle conditions were 95° C. for 2 minutes, 4 cycles of 95° C. for 30 seconds, 68° C. for 30 seconds and 72° C. for 2 minutes, followed by 4 cycles of 95° C. for 30 seconds, 65° C. for 30 seconds and 72° C. for 2 minutes, followed by 30 cycles of 95° C. for 30 seconds, 62° C. for 30 seconds and 72° C. for 2 minutes, followed by 72° C. for 5 minutes and then set to hold at 4° C. PCR reaction products were stored at 4° C. until required.

To observe the amplified products, each PCR was analysed by gel electrophoresis using 1% agarose cast with GelRed (Biotium) and TAE buffer (40 mM Tris, 20 mM acetic acid, and 1 mM EDTA, pH 8). The GoTaq reaction buffer contains dyes, allowing the PCR to be loaded directly onto gels. A total of 10 μl of each PCR was loaded onto the gel along with a 100 bp DNA Step Ladder (Promega). The gel was run at 80 V for 45 minutes before being viewed under UV light in a Gel Doc XR+(Bio-Rad, Hercules, Calif., USA) gel imaging system.

Results

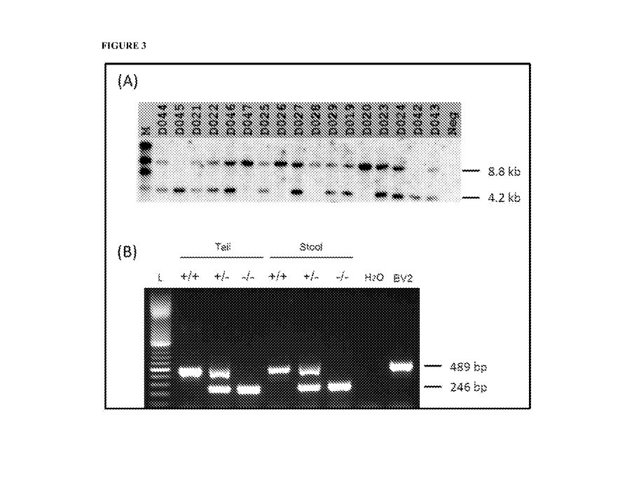

The breeding strategy described was capable of producing offspring that were either TSPO homozygous knock-outs, TSPO heterozygous knock-outs or TSPO wild-type animals, and each of these genotypes could be determined unambiguously by southern blot analysis and/or PCR. Initially, founder animals were assessed by southern blot analysis and FIG. 3(a) shows the genomic DNA of a selection of offspring being detected with a probe directed towards the TSPO exon 4 (See FIG. 1 for annealing site). The presence of the 8.8 kb and 4.2 kb product in lane 2 (DO44) is indicative of a heterozygote, while DO45 (lane 3) is a TSPO homozygous knock-out mouse and DO47 (lane 7) is a wild-type mouse.

FIG. 3(b) shows the results of mice that were genotyped by PCR using the forward and reverse primers. PCR genotyping of a confirmed wild-type mouse (lanes 2 and 5) resulted in a single 489 bp PCR product, while PCR genotyping of a homozygous TSPO knock-out mouse resulted a single 246 bp PCR product. Mice that were heterozygous for the TSPO wild-type allele demonstrated both PCR products. These results were as predicted in FIG. 2.

### Example 2

### Radioligand Membrane Binding

Materials and Methods

Membrane Preparation

Tissue samples were homogenised with T25 digital Ultra-Turrax homogenizer (Ika, Wilmington, N.C., USA) at speed setting 5 or 20000 rpm in approximately 45 mL of ice-cold TRIS buffer (pH 7.4), collected by centrifugation at 48000 g and the supernatant was then discarded. The procedure was then immediately repeated, this acts as an extra wash step to remove any soluble interfering substances to radioligand binding (Byland et al. 1993). Following the second centrifugation and removal ofsupernatant the samples were resuspended in approximately 50 volumes of ice-cold TRIS buffer (pH 7.4). Samples were aliquoted stored in −80° C. until required.

Protein Concentration Measurements

Protein concentration was measured using a bicinchoninic acid (BCA) protein assay kit (Thermo Fisher Scientific, Scoresby, VIC, Australia) following the manufacturer's instructions. In brief BCA Reagent A and BCA Reagent B were combined in a ratio of 50:1 (BCA Reagent A:B) to create a BCA working reagent, protein samples diluted with TRIS buffer were added to the BCA working reagent in a ratio of 20:1 (working reagent: protein). Samples were then incubated for 30 minutes at 37° C. and measured with a spectrophotometer at 562 nm. Standard curves were generated using bovine serum albumin standards provided with the kit.

Saturation Binding

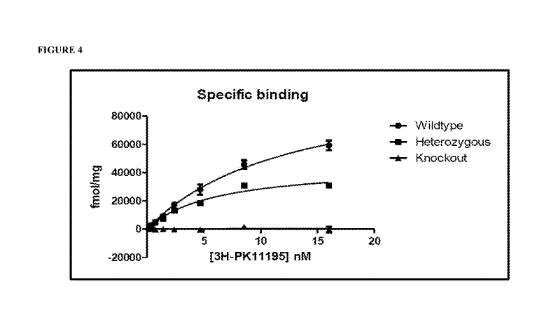

For TSPO knock-out mice studies, radioligand binding was performed using 3H-PK11195 racemate across a range of concentrations (FIG. 4). PK11195 is a TSPO specific probe. Total and non-specific binding was determined through 8 concentrations of 3H-PK11195 ranging from 0.56 nM to 20 nM. Non-specific binding was determined by the addition of 5 μM PK11195 to all concentrations of 3H-PK11195.

The general protocol for radioligand binding involved the preparation of 3H-PK11195 for total binding, 311-PK11195 with PK11195 for non-specific binding and the addition of ice-thawed homogenised protein. 3H-PK11195, PK11195 and protein samples were added to borosilicate glass tubes and if required, protein samples were diluted with TRIS buffer (pH 7.4). Each tube contained 60 μg of protein sample and the final reaction mixture was 400 μL. Each concentration of radioligand in both total and non-specific binding was performed in triplicates.

Protein bound 3H-PK11195 samples were incubated on ice for 90 minutes before harvesting by rapid filtration through glass fibre Whatman GF/C filters (Crown Scientific, Minto, NSW, Australia) pre-soaked in 0.5% polyethylenimine solution. Harvesting was performed by simultaneously washing all tubes with 10 mL of ice-cold TRIS buffer (pH 7.4). The filters were collected and placed in pony vials used for liquid scintillation counting together with 2 mL of Ultima Gold liquid scintillation cocktail (Perkin Elmer, Waltham, Mass., USA). The vials were left at room temperature for at least 12 hours before the amount of radioactivity was determined using a Tri-Carb 2100TR Liquid Scintillation Counter (Perkin Elmer, Waltham, Mass., USA), this was done to allow for sufficient diffusion of the radioactivity into the liquid scintillant (Bylund et al., 1993).

To determine Bmax and Kd values, non-specific binding at the 4 highest concentrations of 3H-PK11195 was estimated by fitting a linear line to the experimentally obtained values. Bmax and Kd values were fitted by non-linear regression using GraphPad Prism version 5.04 for Windows (GraphPad Software, San Diego, Calif., USA) by fitting total and non-specific binding.

Results

Analysis of renal tissues samples from wild-type, TSPO heterozygous and TSPO homozygous mouse can be seen in FIG. 4. Wild-type mice had the highest expression of TSPO, followed by TSPO heterozygous mice. TSPO knock-out mice show no binding of the TSPO-selective probe PK11195.

### Example 3

### Analysis of TSPO Levels in Tissue by Autoradiography

Materials and Methods

Snap-frozen tissues were sectioned at 20 μm in a cryostat, thaw mounted on poly-L-lysine-coated slides and stored at −80° C. until day of experiment. On the day of the experiment, slides were thawed at room temperature and air dried with a cool stream of air. Total and non-specific binding were determined by incubation with 1 nM of 3H-PK11195 with or without the presence of 3 μM of PK11195 in 130 mM TRIS-HCl buffer (pH 7.4) at room temperature for 20 minutes. Following incubation, the slides were briefly dipped twice in 130 mM TRIS-HCl buffer, washed twice for 5 minutes in fresh 130 mM TRIS-HCl at room temperature. The slides were finally briefly rinsed 3 times in chilled distilled H₂O, dried under a cool stream of air and allowed to air dried overnight. Sections were exposed to Kodak BioMax MR film along with tritium microscales with known activity concentrations in X-ray film cassettes. Films were developed after 33 days and digitized using a GS800 Calibrated Densitometer (Bio-Rad, Hercules, Calif., USA). Images were analysed with Quantity One imaging analysis program (Bio-Rad, Hercules, Calif. USA); to assess the level of 3H-PK11195 binding, regions of interest were drawn over the tissue and an average optical density/mm² was calculated. Data obtained from an individual film, subject to interfilm variations, were normalized against other films through the optical density oftritium microscales.

Results

The organs including brain, retina, heart, kidney and testes used for film autoradiography were dissected from wild-type (TSPO^{+/+}), heterozygous (TSPO^{+/-}), and homozygous (TSPO^{-/-}) mice and snap-frozen in liquid nitrogen. Cryostat organ sections at 18 μm were placed onto superfrost slides (Menzel-Glaser, Braunschweig, Germany) and stored in the dark at −20° C. for no longer than 1 week before the experiment. The film autoradiography was performed using R—[N-methyl-³H]PK11195 ethanol solution, with a specific activity of 3.14 TBq·mmol-1 (PerkinElmer, Waltham, Mass., U.S.A.). Tritium standards (Amersham Biosciences, Uppsala, Sweden) co-exposed with the organ sections on each Hyperfilm-³H (Kodak Film) at 4° C. for 60 days, were used to quantify the autoradiographically measured binding. The hyperfilm was developed using Kodak GBX developer and fixer (Sigma Aldrich, St. Louis, Mo., U.S.A.). The hyperfilm was air-dried overnight before being scanned using the ArtixScan 1800f flatbed scanner (Microtek, Hsinchu, Taiwan), with greyscale using a magnification of 400% and an optical resolution of 2400 pixels per inch. No pre-scanning manipulation or filter was applied.

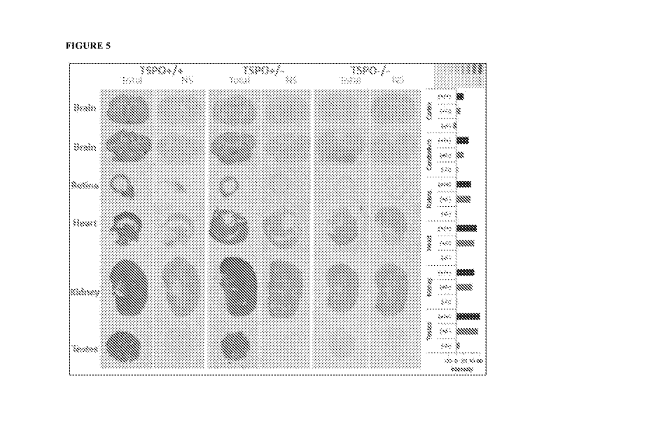

FIG. 5 shows the results of the autoradiography. The images on the left are total binding (specific and non-specific (NS)), while images on the right are non-specific binding. Darker areas represent higher PK11195 binding (i.e. a higher density of TSPO). The bar graph on the right corresponds to the images on left. It shows the highest TSPO expression in wild-type (TSPO^{+/+}) mice in all organs, followed by heterozygous (TSPO^{+/-}) mice, but almost no TSPO expression in homozygous (TSPO^{-/-}) mice. The values shown in the graph are obtained after subtracted background and then subtracted the non-specific binding (NS). The backgrounds selected are the areas outside of organs slices.

### Example 4

### Analysis of Inducible TSPO Levels in Brain Tissue by Autoradiography

Materials and Methods

Procedures for facial nerve axotomy were approved by the animal ethics committee at the University of Sydney. Animals were anaesthetised using Isoflurane anaesthesia. Animals were monitored for a combination of respiration, heart rate, oxygen saturation and temperature during procedures using a variety of monitoring equipment to ensure anaesthetic depth. The surgical process commenced when foot-pad or plantar reflex could no longer be elicited. The fur on the ipsilateral (right) side of the neck was shaved from the ear downwards, and the skin cleaned using 70% ethanol. A small incision (0.5-0.7 cm in length) overlying the musculature covering the stylomastoid foramen was made. The facial nerve was identified, isolated and then transected completely. The skin of incision was closed with heal-glue. The whisker movement ipsilaterally after the operation disappeared and was indicative of success of facial nerve axotomy. The animals were euthanized with CO₂ 3 days after the axotomy and brain tissue, including facial nerve nuclei in the brain stem and cerebellum, was immediately dissected, snap-frozen in liquid nitrogen, and then stored in −80° C. freezer until the cryostat section. Levels of TSPO expression were revealed with film autoradiography using R—[N-methyl-³H]PK11195.

Results

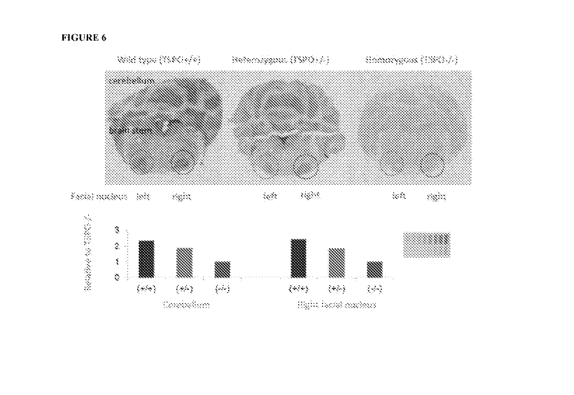

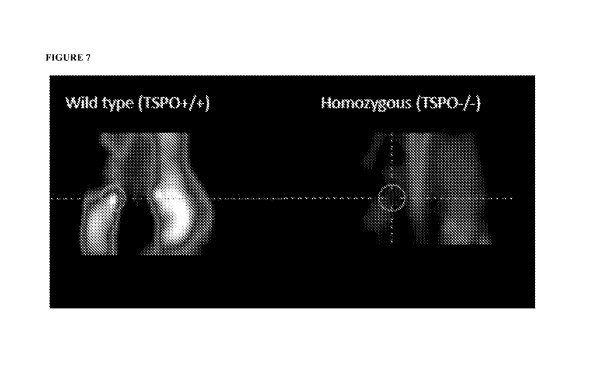

Neuroinflammation in facial nerve nuclei was induced by one-side facial nerve axotomy. Film autoradiographs of the dissected brain tissue are shown in FIG. 6. Facial nerve axotomy induced high levels of TSPO expression in the ipsilateral facial nerve nucleus of wild-type mice (red circle on right-hand side of left image). Lower TSPO expression was observed in heterozygous mice (circle on right-hand side of middle image) while no TSPO was detected in homozygous knock-out mice (circle on right-hand side of right image). It should be noted that the level of TSPO expression was also elevated in the contralateral side of the axotomy in wild-type and heterozygous, but not in homozygous mice (circles on left-hand side of each image).

In the cerebellum, the level of TSPO expression in wild-type mice was high. TSPO expression was not detected in homozygous mice as the intensity of PK11195 binding was the same as the surrounding background (right image, FIG. 6). The level of TSPO expression in heterozygous mice (middle image, FIG. 6) was between the levels observed for wild-type and homozygous mice (left image, FIG. 6).

Experimental models such as the facial nerve axotomy paradigm in rodents allow the systematic and detailed study of the response of neurons and their microenvironment to various types of challenges. Well-studied experimental examples include peripheral nerve trauma, the retrograde axonal transport of neurotoxins and locally enhanced inflammation following the induction of experimental autoimmune encephalomyelitis in combination with axotomy. These studies have led to novel insights into the regeneration programme of the motor neurone, the role of microglia and astrocytes in synaptic plasticity and the biology of glial cells. Importantly, many of the findings obtained have proven to be valid in other functional systems and even across species barriers. In particular, microglial expression of major histocompatibility complex molecules has been found to occur in response to various types of neuronal damage and is now regarded as a characteristic component of “glial inflammation”.

Importantly, the peripheral nerve lesion of the facial nerve leads to the induction of de novo expressed TSPO in the injured facial nucleus, usually unilaterally, but in older animals also bilaterally in both injured facial nuclei. It is found in the context of numerous neurodegenerative disorders including Parkinson's and Alzheimer's disease. The detachment of afferent axonal endings from the surface membrane of regenerating motor neurones and their subsequent displacement by microglia (“synaptic stripping”) and long-lasting insulation by astrocytes have also been confirmed in humans. The medical implications of these findings are significant. Also, the facial nerve system of rats and mice has become the best studied and most widely used test system for the evaluation of neurotrophic factors.

### Example 5

### In Vivo Imaging with Positron Emission Tomography (PET) and Anatomical Information with Computed Tomography (CT)

Materials and Methods

PET scans of test mice were performed using a small-animal Inveon PET/CT scanner (Siemens, Knoxville, Tenn., USA). Before scanning, the mice were anesthetized with 5% (v/v) isoflurane and positioned in the PET/CT scanner. The isoflurane level was maintained 1-2% thereafter. The body temperature was maintained with a feedback regulated heating pad and physiologic parameters (respiration and body temperature) were monitored (BioVet; m2m Imaging Corp.) for the entire scanning period. Lacri-lube (Allergan) was placed in the mouse's eyes to prevent drying while the mouse was anesthetized. The PET scans commenced simultaneously with the tail vein injection of [¹⁸F]PBR111 (8-18 MBq/100 μL, 0.2 nmol). After 40 min of imaging, PBR111 (1 mg/kg in 2% acetic acid-saline) was injected via tail vein to determine the non-specific accumulation in organs and imaged for further 10 minutes. Upon completion of PET scanning, mice were CT scanned for 10 minutes for anatomical information. At the end of the study the mice were euthanized immediately after imaging by cervical dislocation while the animal was still fully anaesthetised.

Results

PBR111 is a ligand that binds to the TSPO protein. FIG. 7 demonstrates that the image on the left shows a high intensity of radiotracer ([18F]-PBR111) recorded in a wild-type mouse (TSPO^{+/+}), while none of the radiotracer was observed in the TSPO knock-out (homozygous, TSPO^{-/-}) mouse on the right. A particular region of interest was the adrenal gland, shown in the circled area, as it has the highest level of TSPO expression in the wild-type mouse. The kidneys can be seen beneath the adrenal gland.

FIG. 9 represents the time course of uptake after radiotracer injection at 0 min and displacement with PBR111 at 40 min in adrenal gland, heart, kidney and liver. The uptake of the radiotracer in the adrenal gland, kidney and liver of the wild-type mice (TSPO+/+) slowly increases, but maintains a peak level until the application of the ligand PBR111, which competes with the radiotracer [18F]PBR111. However, the uptake of the radiotracer in the adrenal gland, kidney, liver and heart of the TSPO knock-out mice (TSPO−/−) rapidly reaches the peak and then declines to the non-specific level (i.e. the values after PBR111). The pattern of radiotracer uptake in the heart of wild-type mice is similar to that of knock-out mice, but the decline is much slower.

### Example 6

### Animal Health Assessment

Material and Methods

Animal Sensory and Reflex Testing

Mice neurological reflexes were examined at 5 months of age. Reflexes were examined one session in the following order: righting, corneal, pinnae, vibrissae, reaching and negative geotaxis reflexes. The presence of reflexes was determined as follows:

Righting reflex: Animals were placed on their back and indications of regaining of ventral down posture was noted.

Corneal reflex: Using a cotton bud, the eye region was lightly stimulated, indications of blinking behaviours was noted.

Pinnae reflex: Using a cotton bud, the auditory meatus was lightly stimulated, indications of ear retraction and head movement were noted.

Vibrissae reflex: Using a cotton bud, the vibrissae were stimulated, head movement, whisker mobility and blinking behaviours were noted.

Reaching reflex: Animals were lowered by the base of their tail slowly towards the surface of a table, extension of forelimbs and hind legs were noted.

Negative geotaxis reflex: Animals were placed head down on inclined plan, tendency to rotate to face uphill was noted.

In addition to reflexes, both vision and audition were examined. Vision was assessed by forepaw reaching during the reaching reflex examination, assessment for vision differs to the reaching reflex in that, the reaching reflex assesses the whole animals posture including the extension of both forelimbs and hind legs, whereas vision is concerned with the active reaching with forepaws. Audition was assessed by clicking of fingers behind an animals and examining for the presence of acoustic startles or freezing behaviour.

Results

The results are summarised below in Table 2. The mice that were homozygous for the TSPO knock-out all appeared normal.

TABLE 2
Results of Animal Sensory and Reflex Testing.

| Phenotype | Stat |
|---|---|
| Breeding - gestation period, litters | Normal |
| sizes, sex | |
| Rate of growth | Normal |
| General activity levels | Normal |
| Reflexes - righting, corneal, pinnae, | Normal |
| vibrissae, reaching and negative | app. |
| Audition | Normal |
| Vision | Normal |
| Grooming & state of fur | Normal |
| Social activity | Normal |
| Food and water consumption | Normal |

### Example 7

### Behavioural Assessment for Anxiety Related Behaviours

Materials and Methods

Behavioural tests for anxiety related behaviours were performed on mice aged between 3-4 months, all mice involved in behavioural assessment were handled regularly after weaning to reduce the impact of handling stress during behavioural testing. Anxiety related behaviours were examined through a battery of behavioural tests and conducted in the following order: open field test, emergence test, light dark preference test and elevated plus maze. All behavioural tests took place between 12:30 p.m. and 6:30 p.m. in a dimly lit room. Mice were taken as a group in their home cages to the experimental room at least 60 minutes prior to testing to allow for sufficient acclimatisation to the experimental environment. To ensure a consistent behavioural testing experience across all animals, animals were promptly returned to their cages and home room once all animals in the cage had been tested. Prior to the start of each test, any urine and stool droppings were removed, the arena cleaned thoroughly with 80% ethanol and allowed to dry before the next test began. In order to reduce the effects of training history, behavioural tests were carried out at least 7 days apart.

All behavioural experiments were recorded with an overhead camera on to DVD and later analysed using Motman Tracker 4.5 software (Motion Mensura, Cooks Hill, NSW, Australia). Motman Tracker allowed for spatial and temporal tracking of movement in real time and from recorded video, allowing for further analysis including regions of interest calculations.

Open Field Test

The open field test measured general locomotor activity as well as anxiety related behaviours. The test consisted of an open square arena surrounded by four walls free from obstacles or objects. The arena measured 44 cm by 44 cm and the walls measured 26 cm in height, both the arena base and walls were constructed from red-tinted acrylic plastic. During the open field test, animals generally tended to explore along the edges of the arena and rarely stayed in the centre where it was more exposed. The amount of time and frequency of crossing the centre of the arena was used as an indication of anxiety.

During the experiment, the arena was evenly lit with red lighting using a shadow-free illumination system. The brightness of the arena was approximately 28 lux, measured using a Testo 545 lux meter (Testo, Croydon South, VIC. Australia). Mice were placed in the centre of the arena and were allowed to freely explore for 15 minutes. The duration and frequency of crossing the centre of the arena, total distance travelled, time spent active and the number of stool droppings were assessed.

Emergence Test

The emergence test was similar to the open field test and utilized similar principles, with the test carried out in an open arena with the addition of a small hide box. As the animal “emerges” from the hide box, the test assesses exploratory behaviours, anxiety and activity levels. The arena measured 44 cm by 44 cm and the walls measured 26 cm in height. The hide box measured 13 by 13 cm with a height of 8.5 cm, the opening of the hide box measured 4 by 4 cm in shape of an arch, clear acrylic panes were attached to the top of the hide box to prevent animals from jumping on top of the hide box during the experiment. The hide box was placed along the centre of one edge in the arena with the entrance facing towards the centre of the arena. The arena and hide box, with the exception of the clear acrylic panes above the hide box, was made from red acrylic plastic.

During the experiment, the arena was evenly lit with a red light using a shadow-free illumination system. The brightness of the arena and hidebox was 28 and 19 lux respectively, measured using a Testo 545 lux meter (Testo, VIC, Australia). Animals were placed into the centre of the arena facing the opening of the hide box and allowed free exploration for 15 minutes. The duration and frequency of entering the hide box, total distance travelled, time spent active and the number of stool droppings were assessed.

Light-Dark Preference Test

The light-dark preference test utilized the observation that rodents find bright light and novel environments mildly stressful. The test used the same arena as in the open field test but separated the arena into a dark and light compartment. The dark compartment was created through the addition of a large box with an opening along the edge to create a passage way. The arena measured 44 cm by 44 cm and the walls measured 26 cm in height. The dark compartment box measured 22 cm by 43.5 cm with a height of 26 cm, the opening of the compartment box measured 4 by 4 cm in shape of an arch. Both the arena and dark compartment box was made from red acrylic plastic.

During the test, the arena was evenly lit with a white light using a shadow-free illumination system, the brightness of the light compartment and dark compartment measured approximately 16 and 3 lux respectively, measured using a Testo 545 lux meter (Testo, Croydon South, VIC, Australia). Mice were placed in the light compartment facing the opening to the dark compartment, and the mice were then allowed free exploration for 15 minutes. The duration and frequency of entering each compartment, total distance travelled, time spent active and the number of stool droppings were assessed

Elevated Plus Maze

The elevated plus maze is a test for anxiety related behaviour and is also able to detect risk assessment behaviours. It is based on the observation that rodents tended to avoid open spaces and remain in enclosed spaces, presumably because it is perceived as a safer environment. The elevated plus maze consisted of 4 long platforms or arms of equidistant connected to a central platform creating a plus-like shape. The arms alternating between being open, no surrounding walls, to closed, walls surround the arm. The arms were elevated at a height of 23 cm and measured 28 cm long and 6 cm wide, the walls of the closed arms measured 20 cm high. The elevated plus maze was constructed from red tinted acrylic plastic and a white plus-shape detachable wooden platform which sat on top of the arms.

During the test, the maze was lit with standard overhead fluorescent lights, the brightness measured approximately 100 lux in the closed arms and 350 lux in the open arms, measured using a Testo 545 lux meter (Testo, Croydon South, VIC, Australia). Mice were placed on the central platform facing the open arms and were allowed free explorations for 15 minutes. Duration and frequency of entering the open arms, duration and frequency of engaging in risk assessment behaviours, total distance travelled, time spent active and the number of stool droppings were assessed. Risk assessment was defined as an animal's head direction was facing towards an open arm with more than 15% of its body in an open arm while maintaining its centre of mass within the closed arm or central platform.

Rotarod Test

The rotarod is a widely used test to assess motor co-ordination and balance. Mice were placed on rods rotating at a fixed or accelerating speed, and the latency to fall from the rod used as a measure for motor co-ordination and balance. TSPO knock-out mice were tested at approximately 5 months of age on the IITC series 8 Rotarod (IITC Life Science, Woodland Hills, Calif., USA) following the European Mouse Phenotyping Resource of Standardised Screens guidelines. Briefly, animals were taken to the experimental room and allowed to acclimatise for 15 minutes before testing. The rotarod test consists of a training session and a testing session. The training session consisted of 3 trials separated by 10 minute rest intervals and the testing session consisted of 4 trials separated by 15 minute rest intervals. The training and testing session was separated by a 30 minute rest interval. The rod rotation direction was set such that animals face away from the experimenter. The rotarod was cleaned and dried at the start of each phase with 70% ethanol.

The training phase consisted of 3 trials each lasting 60 seconds with the rod kept at constant speeds. The rod was kept at 0 rpm for the first trial and at 4 rpm for the second and third trial. Provided that the animals were able to stay on the rod for the fill 60 seconds during the third training trial the animals proceeded to the testing phase following a 30 minute rest interval. If an animal fell off during the third training trial, then an extra training trial (4 rpm at 60 seconds) was given and regardless of performance the animal proceeded to the testing phase following the standard 30 minute rest interval. The testing phase consisted of 4 identical trials each lasting 300 seconds with the rod accelerating from 4 rpm to 40 rpm. Following the last testing trial, animals were returned to their cages and moved back to their home room.

Results

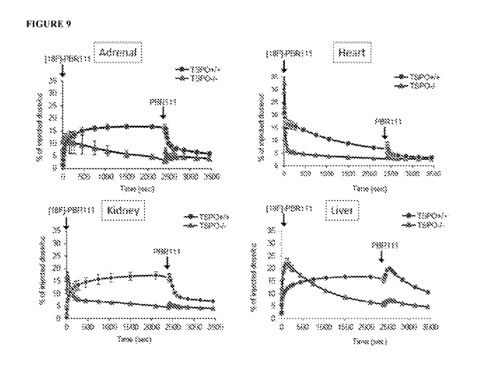

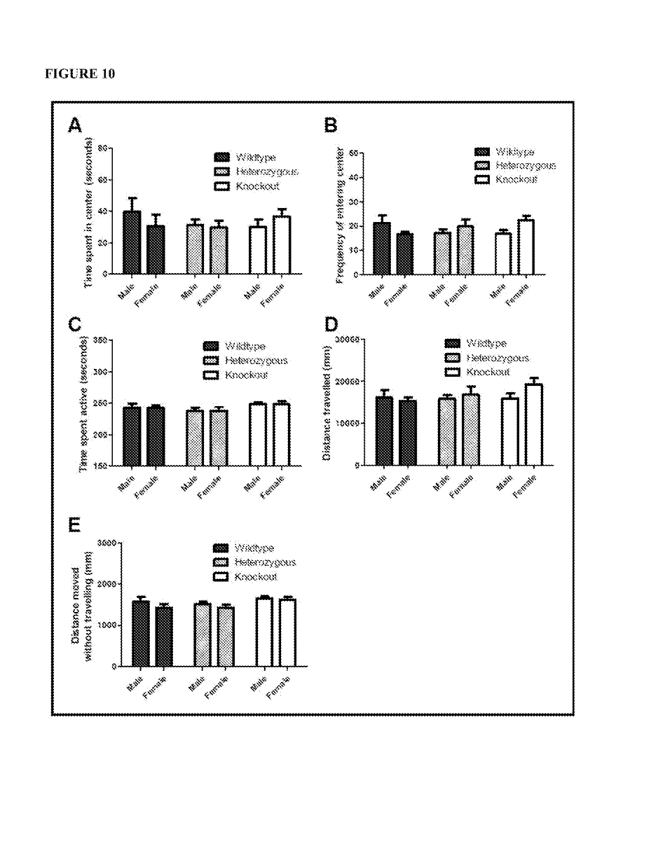

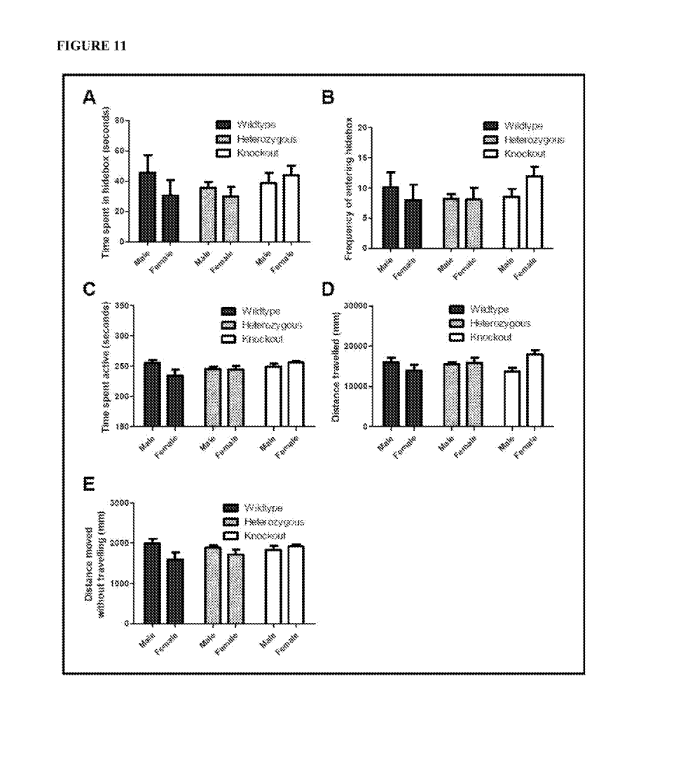

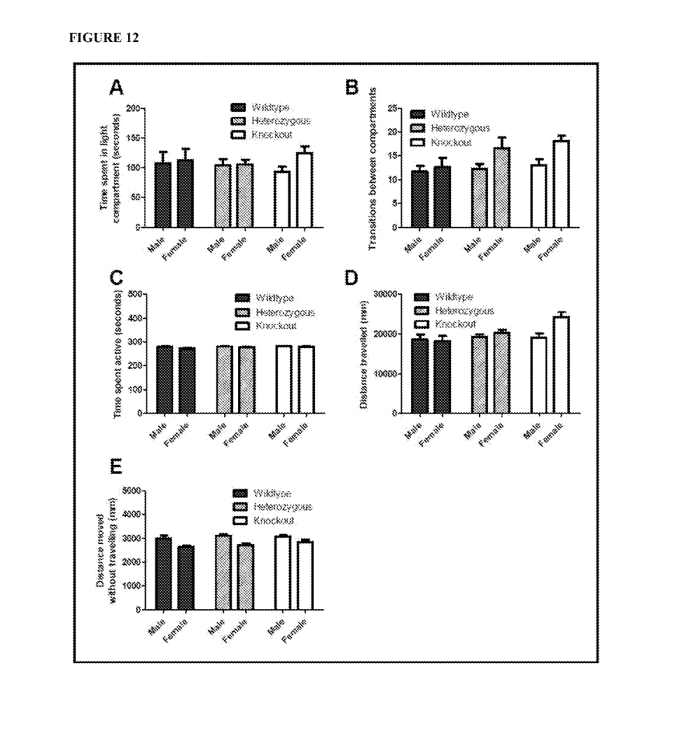

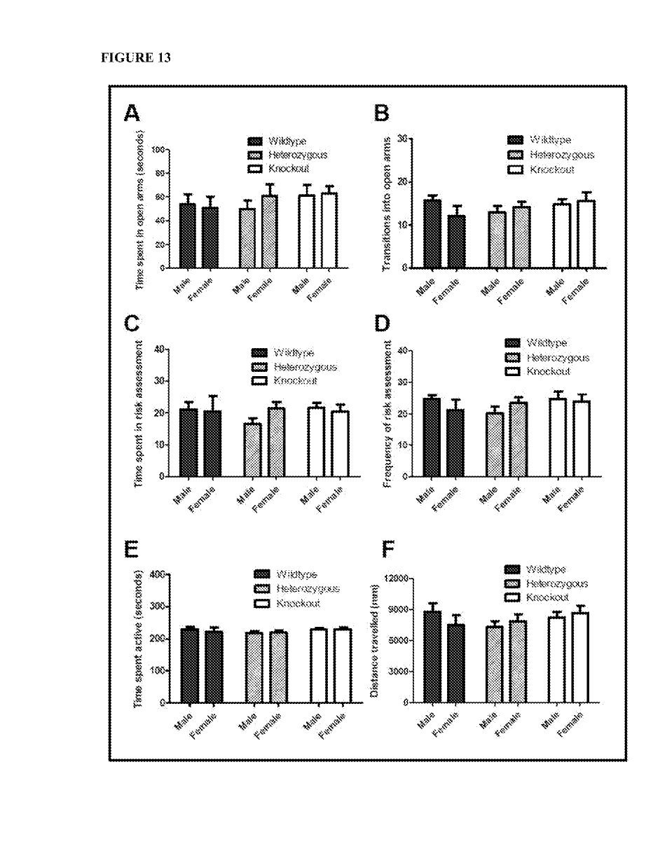

The results of the behavioural assessments for anxiety disorders are illustrated in FIG. 10 to 14. No clear differences between wild-type and TSPO homozygous knock-outs have been reported or observed. Observations of general health, breeding, weaning, growth, activity, genetic distribution of genotypes and distribution of male/female ratios have been the same as the wild-type counterpart. Further, neurological examinations including examination of the righting, corneal, pinnae, vibrissae, reaching and negative geotaxis reflexes have found no obvious deficits in the TSPO knock-out mice. In addition, audition and vision appear to be normal. Detailed examination of basic behaviours such as general activity levels, grooming, state of for, social activity and food/water consumption have all been normal in the TSPO knock-out mice.

### Examples 8 to 13

With reference to Examples 8 to 13 listed below examples a specific section describing the materials and methods relevant for the experiments exemplified is provided here. However, the skilled reader will appreciate that other equivalent methods may be performed to obtain corresponding data.

### Examples 8 to 13

### Materials and Method

Generation of TSPO^{-/-} Mice

TSPO knock-out mice were generated using a targeting construct that contained loxP sites flanking exons 2 and 3, and a neomycin cassette inserted between exons 3 and 4. The targeting construct was electroporated into C57BL/6 Bruce4 embryonic stem (ES) cells and cells correctly targeted by homologous recombination were injected into albino C57BL/6 blastocysts. Male chimeras were mated to female albino C57BL/6 mice and the resulting offspring with a black coat were screened for the presence of a targeted TSPO allele.

Mice positive for the presence of the targeted allele were crossed with C57BL/6 Cre-deleter mice to remove the neomycin cassette and exons 2 and 3 to create heterozygous global-TSPO deficient mice. To remove the Cre transgene, animals were bred to wild-type C57BL/6 mice.

To produce animals for experiments, heterozygous animals were crossed to generate wild-type littermate controls. All animal procedures were approved by the University of Sydney Animal Ethics Committee and the ANSTO Animal Care and Ethics Committee. The TSPO^{-/-} mouse strain has been given the additional designation GuwiyangWurra (‘fire mouse’ in the local Dharawal language). Future naming will thus be C57BL/6-TSPO^{tm1GuMu(GuwiyangWurra)}.

Mice were genotyped by Southern blot analysis using genomic DNA isolated from tail biopsies. For routine genotyping, genomic DNA was isolated from tail biopsies, ear biopsies or stool samples and amplified by PCR using primers P1 (5′-GGTAGACTAGTGTGGGAAGATTTGA), P2 (5′-ATGGTGATTGCAACTGATGTTC) and P3 (5′-TAGATACTGACCCTATCTGGGATGT) to generate a 489 bp product for the wild-type allele and a 246 bp product for the knock-out allele. The PCR consisted of an initial incubation at 95° C. for 2 minutes, followed by 4 cycles at 95° C. for 30 sec, 68° C. for 30 see, and 72° C. for 2 minutes, then 4 cycles at 95° C. for 30 sec, 65° C. for 30 sec, and 72° C. for 2 minutes, then 30 cycles at 95° C. for 30 sec, 62° C. for 30 sec, and 72° C. for 2 minutes and a final step at 72° C. for 5 minutes.

Immunoblotting

Lysates (20 μg protein) were mixed with 2× Laemmli sample buffer (Bio-Rad. Hercules, Calif., USA), heated to 70° C. for 10 minutes and resolved on a 4-20% Mini-PROTEAN TGX gel (Bio-Rad). Proteins were transferred to a nitrocellulose membrane using the Trans-Blot Turbo transfer system (Bio-Rad). The membrane was incubated with anti-TSPO antibody (#109497, Abeam. Cambridge, UK) diluted 1:10000 or anti-GAPDH (#37168. Abcam) diluted 1:1000 and peroxidase-conjugated anti-rabbit antibody (#A0545. Sigma-Aldrich, St Louis, Mo. USA) diluted 1:10000. Pierce ECL Western Blotting Substrate (Thermo Scientific, Scoresby, VIC, Australia) was used and the membrane visualised using an ImageQuant LAS 4000 (GE Healthcare, Rydalmere. NSW, Australia).

RNA Extraction, cDNA Synthesis and Quantitative Real-Time PCR

Tissue samples were homogenised in TRIzol (Life Technologies, Carlsbad, Calif., USA) and total RNA isolated using the PureLink RNA mini kit (Life Technologies) with on-column DNase treatment following the manufacturer's instructions. Purified RNA (1 μg) was reverse-transcribed to cDNA using the SuperScript III First-Strand Synthesis kit (Life Technologies). For quantitative real-time PCR, cDNA was added to 2.5 μl of SsoFastEvaGreen Supermix (Bio-Rad) and 0.5 μM of each primer pair in a final reaction volume of 5 μl. The following primers were used: Actb (forward 5′-GGACCTGACGGACTACCTCATG, reverse 5′-TCTTTGATGTCACGCACGATTT)³²; P450Scc(forward 5′-ACATGGCCAAGATGGTACAGTTG, reverse 5′-ACGAAGCACCAGGTCATTCAC)³; Gapdh (forward 5′-CCATGGAGAAGGCTGGGG, reverse 5′-CAAAGTTGTCATGGATGACC)³⁴; StAR (forward 5′-TCTCTAGTGTCTCCCACTGCATAGC, reverse 5′-TTAGCATCCCCTGTTCGTAGCT)³³; TSPO (forward 5′-GGGAGCCTACTTTGTGCGTGG, reverse 5′-CAGGTAAGGATACAGCAAGCGGG)³⁵; TSPO2 (forward 5′-CCAGTCGGTGTGAGATGAG, reverse 5′-AGTAGAGACCAAGGGGCAGT). Reactions were performed on a CFX 384 Real-Time PCR detection System (Bio-Rad) by cycling at 98° C. for 30 sec, followed by 45 cycles at 98° C. for 5 sec and 63° C. (61° C. for the TSPO2 assay) for 10 sec. A melt curve analysis was performed to confirm the specificity of each reaction. Each sample was run in duplicate. The TSPO2 primers were designed using Primer-BLAST and the specificity confirmed by DNA sequencing.

Animal Weight and Glucose Tolerance Test

Mice were maintained on a standard diet (Rat and Mouse Premium Breeder diet, Gordon's Specialty Stockfeeds) and weights were recorded weekly. To induce obesity, mice at 14 weeks of age were fed a high fat diet (HFD) (SF03-002, 59% of energy from fat) or a control diet (AIN93M, 9% of energy from fat) purchased from Specialty Feeds. Body weight was recorded weekly while food and water intake was measured daily. After 9 weeks on the control or HFD, mice underwent glucose tolerance tests. After 6 hours fasting, blood from the tail vein was measured using an AlphaTRAK 2 glucometer (Abbott Laboratories, Abbott Park, Ill., USA). Mice received an intraperitoneal glucose injection at 2 g/kg of body weight and blood glucose was measured at 15, 30, 60 and 120 minutes thereafter.

Protoporphyrin IX

Blood from euthanized mice was placed into heparin tubes on ice, plasma removed, cells washed twice in ice-cold phosphate buffered saline (PBS) and then resuspended in PBS to the original volume. Samples were evenly split (300 μl each), one receiving 1 mM 5-ALA and the other sterile H₂O, and incubated in a shaking incubator at 37° C. for 4 hours. Ethyl acetate/acetic acid (4:1) was added to each sample, then mixed and centrifuged before transferring the supernatant (1 mL) to a new tube with 1 mL of 1.5 M HCl. An aliquot of the lower HCl phase, after mixing and centrifuging the sample, was measured in a spectrofluorimeter (excitation wavelength was 407 nm, slit width 10 nm, emission spectrum 450 to 800 in 1 nm increments). Glass tubes and containers were used throughout and samples were protected from light.

Facial Nerve Axotomy

Facial nerve axotomy (Banati, R. B., Myers. R. & Kreutzberg, G. W. PK (‘peripheral benzodiazepine’)-binding sites in the CNS indicate early and discrete brain lesions: microautoradiographic detection of [3H]PK11195 binding to activated microglia. J Neurocytol 26, 77-82 (1997); Moran, L. B. & Gracher, M. B. The facial nerve axotomy model. Brain Res Brain Res Rev 44, 154-178, doi:10.1016/j.brainresrcv.2003.11.004 (2004)) was performed on TSPO^{+/+} and TSPO^{-/-} mice anaesthetized with isoflurane. A small incision (0.5-0.7 cm) in the skin 0.5 cm dorsal and lateral to the ear was made and the facial nerve transected under an operating microscope. The incision was closed with 3M™ Vetbond™ Tissue Adhesive (St. Paul, Minn., USA). Success of the operation was confirmed by the loss of ipsilateral whisker movement. Mice were monitored carefully and euthanized with CO₂ three days after the operation. Brains were dissected, placed in OCT, transferred to liquid nitrogen, and stored at −80° C. until cryo-sectioning.

PET and CT Imaging

Mice, anaesthetised (5% (v/v) isoflurane and maintained at 1-2%, were scanned using a small-animal Invcon PET/CT scanner (Siemens, Knoxville, Tenn., USA) according to methods described previously (Disselhorst, J. A. et al. Image-quality assessment for several positron emitters using the NEMA NU 4-2008 standards in the Siemens Inveon small-animal PET scanner. J Nucl Med 51, 610-617, doi:10.2967/jnumed.109.068858 (2010); Mattner, F. et al. Central nervous system expression and PET imaging of the translocator protein in relapsing-remitting experimental autoimmune encephalomyelitis. J Nucl Med 54, 291-298, doi: 10.2967/jnumed.112.108894 (2013)). Body temperature was maintained with a feedback regulated heating pad and respiration monitored (BioVet; m2m Imaging Corp, Cleveland, Ohio, USA). Scans started with the tail vein injection of [¹⁸F]PBR111 (8-18 MBq/100 μL, 0.2 nM). After 40 min of imaging, PBR111 (1 mg/kg in 2% acetic acid-saline) was injected to determine non-specific accumulation in organs and imaged for 10 minutes. Then, mice underwent a 10-min CT-scan for anatomical information. All PET data were corrected, normalised and reconstructed with an OSEM3D-MAP algorithm to produced PET volumes of activity concentration (kBq/ml).

Autoradiography

Tissue sections from TSPO^{+/+}, TSPO^{+/-} and TSPO^{-/-} mice were incubated at room temperature for 20 min in 170 mM Tris-HCl pH 7.4 containing 1 nM [³H]PK11195 (specific activity 84 Ci/mmol; Perkin Elmer, Waltham, Mass., USA), washed twice for 5 min in 170 mM Tris-HCl, rinsed with 3 dips in ice-cold MilliQ H₂O, and dried. Additional sections were incubated on ice for 1 hr in 50 mM Tris-HCl pH 7.4 containing 3 nM [¹²⁵I]CLINDE (specific activity 100 Ci/mmol; synthesised by ANSTO Life Sciences), washed twice for 2 min in ice-cold 50 mM Tris-HCl, for 1 min in ice-cold MilliQ H₂O, and dried. Displacement binding was carried out in the presence of PK11195 (10 μM), CLINDE (10 μM) and PBR-11 (10 μM). Single-emulsion films were exposed to sections and standards (¹⁴C or ³H) for 3 hours ([¹²⁵I]CLINDE) or 10 weeks ([³H]PK11195).

Immunohistochemistry

Immunohistochemistry was performed as previously described (Banati, R. B. et al. The peripheral benzodiazepine binding site in the brain in multiple sclerosis: quantitative in vivo imaging of microglia as a measure of disease activity. Brain 123 (Pt 11), 2321-2337 (2000); Gracber, M. B., Streit, W. J. & Kreutzberg, G. W. Axotomy of the Rat Facial-Nerve Leads to Increased Cr3 Complement Receptor Expression by Activated Microglial Cells. J Neurosci Res 21, 18-24, doi:DOI 10.1002/jnr.490210104 (1988); Liu, G. J., Nagarajah, R., Banati, R. B. & Bennett, M. R. Glutamate induces directed chemotaxis of microglia. European Journal of Neuroscience 29, 1108-1118, doi:10.1111/j.1460-9568.2009.06659.x (2009)). Tissue sections (used for autoradiography) were fixed with 3.7% formaldehyde in PBS for 5 min then permeabilised with ice-cold acetone. Non-specific binding was blocked with 10% horse serum and 2% BSA in PBS. Sections were incubated with TSPO monoclonal antibody (Abcam #109497) at 4° C. overnight and secondary HRP-conjugated goat anti-rabbit antibody at RT for 1 hour (Sigma #A0545). The activity of HRP was detected with the peroxidase 3,3′-diaminobenzidine tetrahydrochloride liquid substrate system (Sigma-Aldrich). Sections were dehydrated with ethanol, incubated with xylene, and slides were mounted in D.P.X. mounting media with a cover slip. Sections were visualized under an inverted Olympus BX51 microscope (Olympus, Tokyo. Japan), and captured with a Q-imaging camera and ImagePro 5.1 program.

A Zeiss LSM 710 confocal microscope with a 5× EC Plan-Neofluar NA 0.16 objective was used for the fluorescence microscopy of the facial nucleus. Alexa Fluor 568 was excited with a 561 nm laser, using a 488/561/633 dichroic mirror, and emission captured from 565 to 640 nm.

For immunocytochemistry, peritoneal macrophages were isolated according to Zhang et al. (The isolation and characterization of murine macrophages. Curr Protoc Immunol Chapter 14. Unit 14 11, doi:10.1002/0471142735.im1401s83 (2008)). After 2 days in completed cultural medium the purity of macrophages was >97% as confirmed with the macrophage specific marker IB4 conjugated with FITC (Sigma-Aldrich). Cells on cover slips were fixed and incubated using the same method as for tissue sections, with the addition of a mouse anti-human/mouse mitochondrial electron transport chain complex IV antibody (Abeam AB14705), then incubated with the secondary antibodies Alexa Fluor (AF) 594-conjugated goat anti-rabbit antibody (Life Technologies) and AF488-conjugated goat anti-mouse antibody (Life Technologies). Cells on cover slips were mounted with ProLong Gold antifade reagent containing DAPI (Life Technologies) and viewed under a BX61 WI Olympus microscope. Images were acquired with a digital camera (CoolSNAP, Photometrics, Tucson, Ariz. USA) and the Image InVivo program (Photometrics). Deconvolution of images was performed with the AutoDeblur program (Photometrics) and further processed with ImagcJ (NIH, Baltimore, Md., USA).

Primary Microglial Cell Culture

Microglia were cultured from 0 to 2-day-old TSPO^{+/+} and TSPO^{-/-} mice according to methods described previously (Liu, G. J., Nagarajah, R., Banati, R. B. & Bennett, M. R. Glutamate induces directed chemotaxis of microglia. Eur J Neurosci 29, 1108-1118, doi:EJN6659 [pii]10.111/j.1460-9568.2009.06659.x (2009)). Briefly, whole brains were dissected, cut into small pieces and treated with 0.025% trypsin (Sigma-Aldrich). They were cultured in Dulbocco's modified Eagle's medium (DMEM/F2; Sigma-Aldrich) supplemented with 10% fetal bovine serum (FBS; Life Technologies), 1% penicillin-streptomycin-glutamine (Sigma-Aldrich) and 0.5 ng/ml GM-CSF (Abcam).

Microglia were purified by shaking at 350 rpm for 50 min at 37° C., pelleted by centrifugation, resuspended in supplemented DMEM/F12 and placed (4×10⁴ cells/well) into a 96 well Seahorse XF cell culture plate (Seahorse Bioscience, North Billerica, Mass., USA) pre-coated with FBS for 15 min at room temperature. After 15 min the wells were washed twice to remove unattached cells. The purity of microglial cultures was >99% as confirmed by staining with the microglial marker Alexa Fluor-conjugated isolectin GS-IB4 (Life Technologies). Microglia were grown for 2-3 days before evaluating mitochondrial functions with the Seahorse XF 96 Analyzer (Seahorse Bioscience).

Measurements of Mitochondrial Respiration

The oxygen consumption rate (OCR) and extracellular acidification rate (ECAR) in primary microglial cultures were determined using the Seahorse XF 96 Analyzer.OCR is an indicator of mitochondrial respiration, and ECAR is primarily the result of glycolysis (Brand, M. D. & Nicholls, D. G. Assessing mitochondrial dysfunction in cells. The Biochemical journal 435, 297-312, doi:10.1042/BJ20110162 (2011); Yadava, N. & Nicholls. D. G. Spare respiratory capacity rather than oxidative stress regulates glutamate excitotoxicity after partial respiratory inhibition of mitochondrial complex I with rotenone. J Neurosci 27, 7310-7317, doi:10.1523/JNEUROSCI.0212-07.2007 (2007)). The day before the experiment 200 μL of the Seahorse XF Calibrant (pH 7.4) was added to each well of the XF 96 well utility plates. The sensor cartridge was placed on top of the plate and hydrated for 16 hours at 37° C. in a non-CO₂ incubator. The protocol for basal measurements of the cells at 37° C. (start, calibrate probes, equilibrate, mix 2 minutes, 1 minute delay, measure 3 minutes) was repeated twice. Mitochondrial stress compounds (oligomycin, FCCP, rotenone and antimycin A) were then injected followed by: 2 minutes mixing, 1 minute delay, measure 3 minutes (repeated 3 times).

On the day of the experiment microglia, purity >98% and viability >92% for both wild-type and knock-out microglia, were washed 3 times with a un-buffered DMEM plus 25 mM glucose (no buffers) and incubated at 37° C. (no CO₂) for 1 hour. Four tests were run on the Seahorse XF 96 Analyzer, each using four drugs (oligomycin, FCCP, and rotenone combined with antimycin A), chosen to examine four key parameters of the mitochondrial electron transport chain: basal respiration. ATP production, proton leak, and maximal respiration (Brand, M. D. & Nicholls, D. G. Assessing mitochondrial dysfunction in cells. The Biochemical journal 435, 297-312, doi:10.1042/BJ20110162 (2011); Yadava, N. & Nicholls, D. G. Spare respiratory capacity rather than oxidative stress regulates glutamate excitotoxicity after partial respiratory inhibition of mitochondrial complex I with rotenone. J Neurosci 27, 7310-7317, doi: 10.1523/JNEUROSCI.0212-07.2007 (2007)) (FIG. 4A). Oligomycin and the combination of rotenone and antimycin A were injected at concentrations of 0.3 μM, 1 μM, 3 μM (optimal) and 10 μM. FCCP was injected at concentrations of 0.1 μM (optimal), 0.3 μM, 1 μM and 3 μM.

Radioligand Binding

Organs were homogenised, washed twice in ice-cold 50 mM Tris-HCl (pH 7.4) by centrifugation at 48000×g, resuspended in 50 volumes of ice-cold 50 mM Tris-HCl, and protein concentration was determined using a BCA protein assay kit (Thermo Fisher Scientific). Bmax and Kd were determined with saturation binding by incubating membrane protein (60 μL) with [³H]PK11195 (0.56 nM-20 nM) on ice for 90 minutes before harvesting by rapid filtration through Whatman GF/C filters (GE Healthcare Life Science, Australia) pre-soaked in a 0.5% polyethylenimine solution. Non-specific binding was determined with 5 μM PK11195. Filters were placed in scintillation cocktail (Perkin Elmer) for 12 hours and radioactivity was determined using a Tri-Carb 2100TR Liquid Scintillation Counter (Perkin Elmer). Bmax and Kd values were obtained by non-linear regression using GraphPad Prism 5.04 (GraphPad Software, La Jolla, Calif., USA) by fitting total and non-specific binding.

Cholesterol Transport and Biosynthesis

P450Scc metabolism was measured in mouse testis according to Tuckey (Tuckey, R. C., Woods, S. T. & Tajbakhsh, M. Electron transfer to cytochrome P-450scc limits cholesterol-side-chain-cleavage activity in the human placenta. European journal of biochemistry/FEBS 244, 835-839 (1997)) and Slominski (Slominski, A. T. et al. Cytochrome P450scc-dependent metabolism of 7-dehydrocholesterol in placenta and epidermal keratinocytes. The international journal of biochemistry & cell biology 44, 2003-2018, doi:10.1016/j.biocel.2012.07.027 (2012)). Briefly, an enriched mitochondrial fraction was prepared by centrifugation in 250 mM sucrose, 50 mM Tris (pH7.4), and the enzymatic conversion of cholesterol to pregnenolone was conducted under the following conditions out to a maximum 2 hour time-point: 50 mM HEPES pH7.4, 250 mM sucrose, 20 mM KCl, 5 mM MgSO₄, 0.2 mM EDTA, 1 mg/ml BSA, 8 μM Trilostane, 0.05 μCi³H-cholesterol, 500 mM Isocitrate, 5 mM NaNADP. Reactions were terminated with the addition of 4° C. dichloromethane. The organic phase was retained and the aqueous phase was re-extracted twice more into dichloromethane. Extracts were combined, dried under nitrogen, and dissolved in 100 μl of ethyl acetate. Pregnenolone and cholesterol were separated by thin layer chromatography, with hexane:diethylether:acetic acid (15:15:1) mobile phase, and the amount of radioactivity in each spot quantified by liquid scintillation counting.

Detection of Serum Pregnenolone

Serum pregnenolone of male and female TSPO^{+/-}- and TSPO^{+/+} mice was determined by enzyme-linked immunosorbent assay (ELISA) performed according to the manufacturer's instructions (Abnova Cooperation, Taiwan). Briefly, blood was collected, allowed to clot, and the serum collected. Standards, controls and samples were incubated in duplicate in a sealed plate with or without conjugate and antibody for 1 hour at room temperature on a plate shaker (KS4000ic, IKA. Selangor, Malaysia) at 200 rpm. After the incubation, wells were emptied, washed 5 times, and the plate tapped on lint free paper towel to remove any remaining buffer. Wells were incubated with HRP conjugate on the plate shaker at 200 rpm for 30 minutes at room temperature, washed 5 times, and incubated with TMB substrate for 10 min at room temperature. Stop solution was added to each well and after 20 min the optical density at 450 nm was measured. Data were normalized against blood volume and analysed using a 4 parameter logistic curve fitting program MasterPlex ReaderFit: Curve-Fitting Software for ELISA Analysis (Hitachi Solution America, San Francisco, Calif., USA).

Blood Phenotyping

Thirty mice (10 TSPO^{+/+}, 10 TSPO^{+/-}, 10 TSPO^{-/-}, 5 months old, males/females evenly distributed) used in behavioural experiments were euthanized with CO₂. Blood was collected from the pulmonary cavity by cutting the inferior vena cava and placed in microtubes with heparin (haematology and lymphocyte analysis) or gel clot activator (biochemical analysis). Spleens were collected in 10 mL of ice-cold sterile PBS. Blood (room temperature) and spleens (on ice) were delivered (within 3-7 hours of collection) to the Australian Phenomics Facility for flow cytometric and biochemical analysis.

For general haematology, blood was diluted 1:2 and analysed with fluorescence-activated cell sorting (FACS) using the Advia 2120 Haematology System (Siemens, Munich. Germany). Blood cell subsets, including white blood cells and platelets, were assessed. For lymphocyte analysis, blood was analysed for the presence or absence of abnormalities in T cells, B cells, NK cells and monocytes. The T cell subsets included CD4+ and CD8+ subsets, while the B cell subsets included immature and mature B cells and IgE+ monocytes. Spleens were analysed by flow cytometry and stained for NK cells, for B cells, including mature and immature subsets, and for T cells, including CD4+ and CD8 cells and their activated subsets. See Table 1 for a list of cell types examined.

For biochemistry, blood was centrifuged, the serum collected and run through the Olympus AU400 Chemistry Analyzer (Olympus), which detects levels of cholesterol triglycerides, glucose, high-density lipoprotein (HDL), albumin. creatine kinase activity and alanine aminotransferase activity. See Table 1 for a list of biochemical parameters examined.

Mouse Sperm Analysis

Sperm were released from the caudaepididymides of TSPO^{+/+} and TSPO^{-/-} mice by cutting each cauda with a scalpel and incubating in 150 μl of pre-warmed Biggers-Whitten-Whittingham (BWW) medium for 5 minutes at 37° C. After the incubation, an additional 250 μl of BWW medium was added, the tissue removed and the sperm assessed using a computer assisted sperm analysis (CASA) system (Hamilton Thorne, Beverly, Mass., USA). Parameters assessed were total motile, progressive motile, average path velocity (VAP), straight line velocity (VSL) and curvilinear velocity (VCL).

Assessment of Anxiety Related Behaviours

Behavioural tests were performed on mice aged between 3-4 months, handled regularly to reduce the impact of handling stress during testing. Anxiety related behaviours were examined with the following: open field test, emergence test, light/dark preference test and elevated plus maze. In each test the mice were allowed free exploration in the apparatus for 15 min. The duration and frequency of entering each compartment, total distance travelled, time spent active and the number of stool droppings were assessed. In the elevated plus maze risk assessment was defined as when an animal's head was facing towards an open arm with more than 15% of its body in an open arm while maintaining its centre of mass within the closed arm or central platform. The genotype of the animals was blind to the experimenter during testing. Tests were carried out at least 7 days apart to reduce the effects of training history (Paylor, R. & Lindsay. E. Mouse models of 22q11 deletion syndrome. Biol Psychiatry 59, 1172-1179, doi:10.1016/j.biopsych.2006.01.018 (2006)). All experiments were recorded with an overhead camera on to DVD and later analysed using Motman Tracker 4.5 software (Motion Mensura, Cooks Hill, NSW, Australia).

Statistics

Results presented are from at least 3 wells and batches of microglial cultures, where (n) refers to number of wells sampled for mitochondrial respiration. At least 3 animals per group were used in other experiments. Data are presented as meant ±SEM, except PET quantification and cholesterol transport data where SD is given. Comparisons between groups were made with a univariate analysis of variance with a Bonferroni's post-hoc test. P values ≦0.05 were considered statistically significant.

### Example 8

### Confirmation of Global TSPO^{+/-} and TSPO^{-/-} Knock-Out

Results

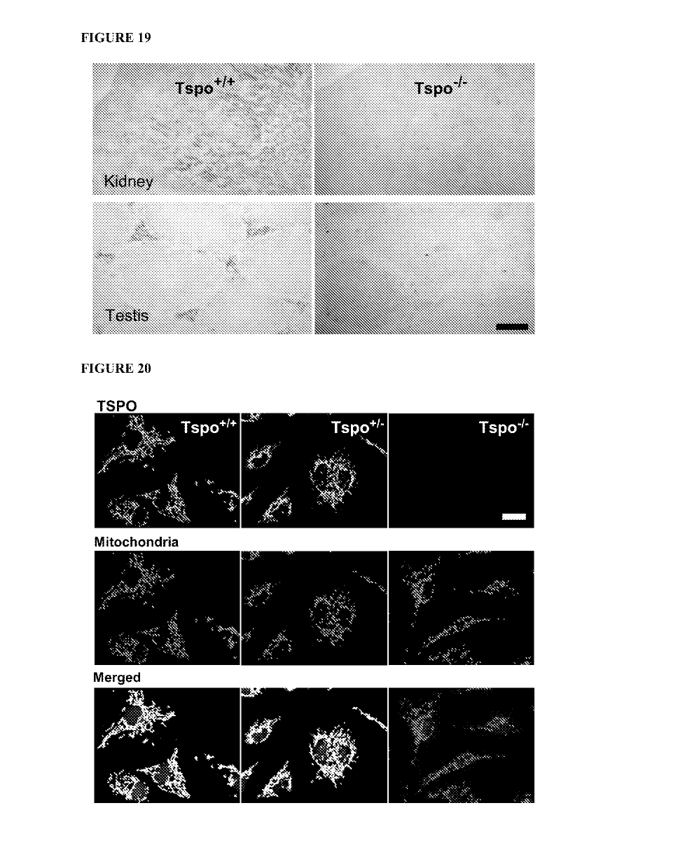

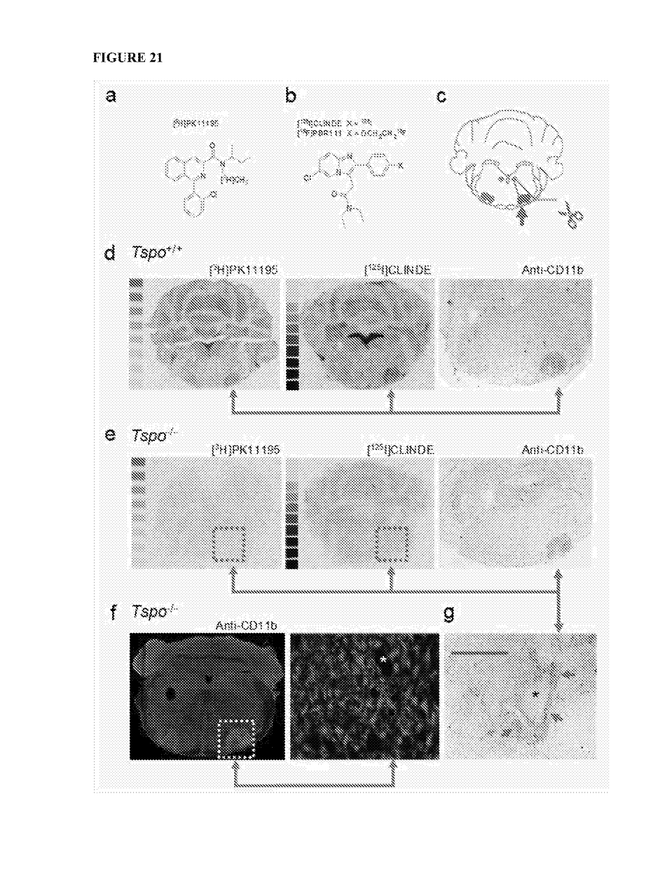

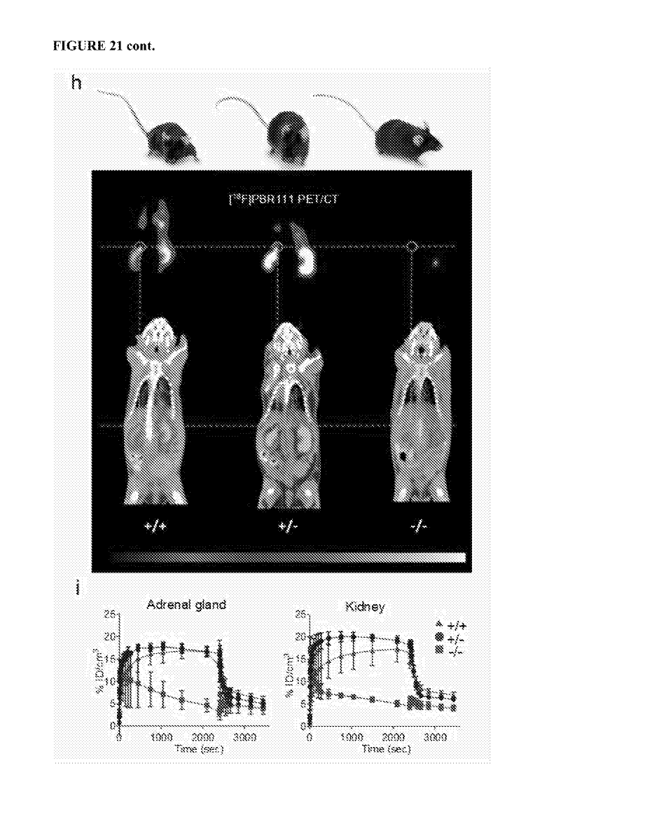

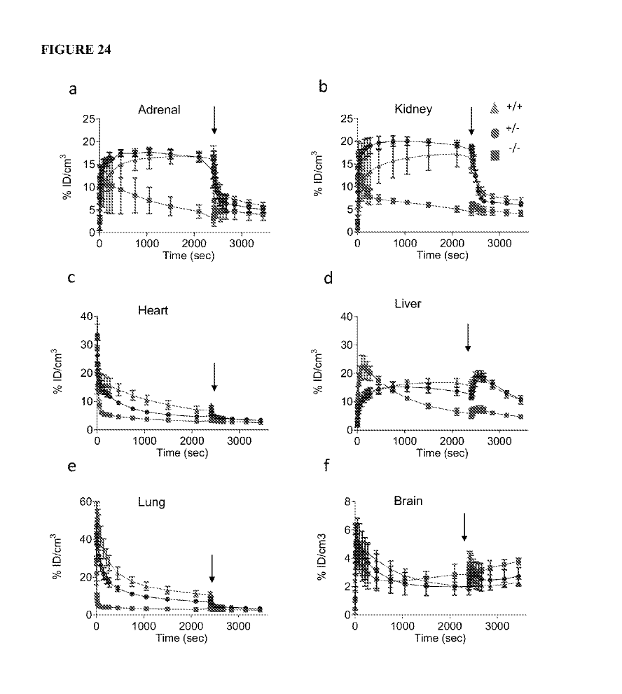

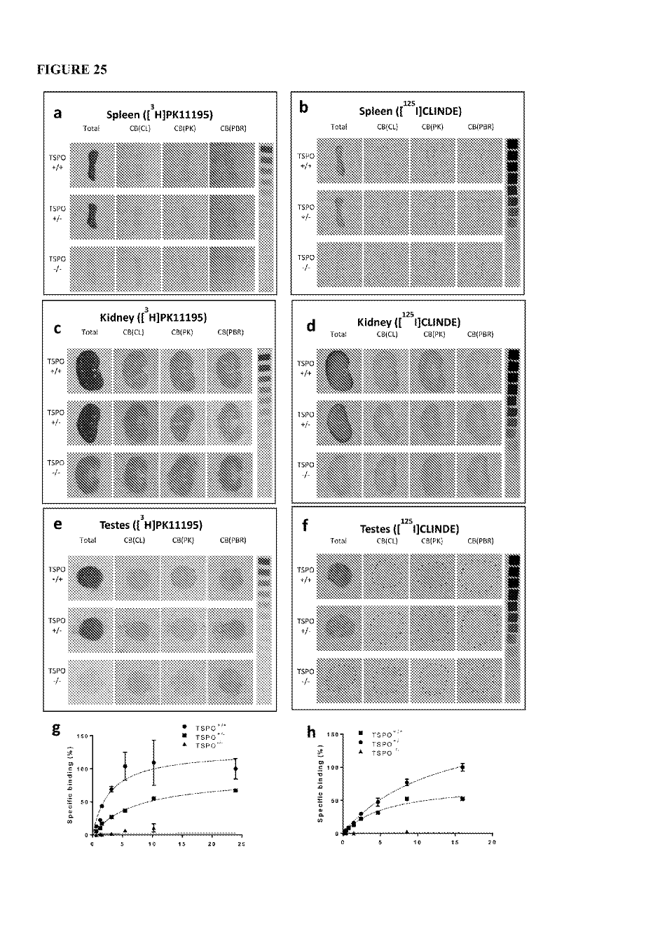

Removal of exons 2 and 3 of the TSPO gene resulted in viable TSPO^{+/-} and TSPO^{-/-} animals. The disruption of TSPO was confirmed by Southern blot, PCR, RT-qPCR, Western blot, (FIGS. 15 to 18), specific antibody staining (FIGS. 19, 20 and 21a to 21g), in vivo tracer kinetic PET/CT studies using the TSPO ligand [¹⁸F]PBR111 (FIGS. 21h and i; FIG. 24), receptor-autoradiography and membrane receptor binding using [³H]PK11195 and [¹²⁵I]CLINDE (FIGS. 21a and 21b, respectively).

In addition to confirming the absence of the TSPO protein, our data demonstrate the high selectivity of [³H]PK11195, the molecule originally used to pharmacologically characterise the peripheral benzodiazepine binding site¹⁸. Further, we demonstrate in vivo and in vitro the high selectivity of [¹⁸F]PBR111 and [¹²⁵I]CLINDE (FIGS. 21a to 21g), which are thus the first new compounds for the TSPO validated in animals with a null background of any constitutive, or induced, specific TSPO binding.

Importantly, we show that in TSPO^{-/-} animals, unlike in the normal wild-type, the microglial cell response in the facial nucleus after peripheral facial nerve lesion is not associated with an increase in the binding of the TSPO ligands, [³H]PK11195 and [¹²⁵I]CLINDE. This demonstrates that in pathologic tissue changes, the selectivity of [³H]PK11195 and [¹²⁵I]CLINDE holds true and no additional non-selective binding emerges. Our data also indicate that the early stage of perineuronal microglial activation, with its typical change in microglial morphology, is not noticeably influenced by the loss of the TSPO and that the neuro-glial signalling mechanism remains intact (FIGS. 21f and 21g).

### Example 9

### General Health, Viability, Fertility and Behavioural Phenotyping

Results

The observation of over 600 animals did not reveal any overt clinical impairment under normal feeding and housing conditions, nor increase in incidental pathologies in either heterozygous TSPO^{+/-} or homozygous TSPO^{-/-} animals, both having the same gender ratio, growth rate and weight increase over time as the colony control wild-type TSPO^{+/+} animals (FIG. 25). Likewise, there was no indication of a decrease in fertility or lifespan, the oldest animals having so far exceeded 20 months without illness. Tests of sperm viability and function yielded no differences between sperm from TSPO^{+/-} (mean motility: 82.7±9.33%; mean progress: 47.7±10.2%; mean velocities VAP: 98.6±13.0 μm/s, VSL: 65.7±8.90 μm/s, and VCL: 179±20.2 μm/s) and TSPO^{-/-} animals (mean motility: 86.1±5.74%; mean progress: 49.6±5.32%; mean velocities VAP: 105±6.54 μm/s, VSL: 67.5±5.08 μm/s, and VCL: 197±11.2 μm/s). Standard open field, emergence, light/dark preference and elevated plus maze tests revealed similar behaviour across all genotypes (Table 3).

TABLE 3
Behaviour phenotyping

| Test | index | gender | TSPO^{+/+} | TSPO^{+/-} | TSPO^{-/-} |
|---|---|---|---|---|---|
| Open field | Time spent in centre | Male | 160.1 ± 101.1 | 87.70 ± 36.35 | 112.3 ± 43.01 |
| | (sec) | Female | 94.33 ± 24.28 | 124.0 ± 45.96 | 99.94 ± 33.94 |
| | Frequency of entering | Male | 60.57 ± 23.78 | 49.75 ± 16.61 | 53.42 ± 10.40 |
| | centre | Female | 48.83 ± 10.89 | 53.75 ± 14.83 | 58.64 ± 13.92 |
| | Time spent active (sec) | Male | 711.0 ± 50.49 | 704.0 ± 53.59 | 738.0 ± 26.08 |
| | | Female | 714.3 ± 31.12 | 676.4 ± 42.13 | 725.7 ± 38.34 |
| | Distance travelled (mm) | Male | 43180 ± 11069 | 44381 ± 10006 | 42084 ± 7590 |
| | | Female | 44266 ± 7782 | 39516 ± 10048 | 50333 ± 12334 |
| | Distanced moved | Male | 4308 ± 758.1 | 4185 ± 739.5 | 4693 ± 556.8 |
| | without travelling (mm) | Female | 4092 ± 584.8 | 3729 ± 476 | 4444 ± 603.8 |
| Emergence | Time spent in hidebox | Male | 155.4 ± 42.56 | 129.1 ± 35.64 | 159.8 ± 56.15 |
| test | (sec) | Female | 121.8 ± 41.59 | 113.1 ± 44.85 | 121.1 ± 43.18 |
| | Frequency of entering | Male | 30.28 ± 15.22 | 25.55 ± 6.629 | 26.42 ± 9.020 |
| | hidebox | Female | 25.17 ± 9.411 | 23.00 ± 10.50 | 28.55 ± 9.903 |
| | Time spent active (sec) | Male | 751.9 ± 25.91 | 746.2 ± 31.06 | 752.1 ± 32.88 |
| | | Female | 705.7 ± 38.97 | 711.9 ± 35.08 | 748.3 ± 32.77 |
| | Distance travelled (mm) | Male | 44545 ± 4826 | 45864 ± 6128 | 41474 ± 5881 |
| | | Female | 41125 ± 7451 | 42068 ± 8191 | 49188 ± 8525 |
| | Distance moved without | Male | 5765 ± 528.4 | 5675 ± 673.2 | 5631 ± 784.8 |
| | travelling (mm) | Female | 4781 ± 730.2 | 4753 ± 641.5 | 5335 ± 676.8 |
| Light/dark | Time spent in light | Male | 382.1 ± 115.7 | 422.8 ± 112.5 | 374.9 ± 76.04 |
| preference | compartment (sec) | Female | 418.2 ± 84.50 | 372.0 ± 85.95 | 412.9 ± 113.0 |
| test | Transition between | Male | 32.00 ± 6.403 | 36.67 ± 11.66 | 33.08 ± 8.229 |
| | compartments | Female | 41.33 ± 10.29 | 46.62 ± 19.64 | 46.09 ± 9.148 |
| | Time spent active (sec) | Male | 819.4 ± 25.79 | 821.7 ± 15.86 | 827.5 ± 12.60 |
| | | Female | 803.8 ± 20.20 | 797.0 ± 19.08 | 810.5 ± 17.62 |
| | Distance travelled (mm) | Male | 50219 ± 5948 | 52916 ± 5955 | 50071 ± 6780 |
| | | Female | 53074 ± 9692 | 48749 ± 6220 | 58633 ± 9674 |
| | Distance moved without | Male | 8363 ± 943.2 | 8367 ± 790.1 | 8359 ± 532.8 |
| | travelling (mm) | Female | 7682 ± 443.0 | 7219 ± 606.8 | 7712 ± 735.3 |
| Elevated | Time spent in open arms | Male | 134.6 ± 59.75 | 122.9 ± 75.71 | 145.1 ± 59.14 |
| plus maze | (sec) | Female | 103.8 ± 55.92 | 139.2 ± 78.00 | 152.46 ± 87.09 |
| | Frequency of entering | Male | 33.14 ± 8.783 | 28.29 ± 13.83 | 29.50 ± 7.205 |
| | open arms | Female | 25.50 ± 11.15 | 28.62 ± 11.35 | 31.55 ± 17.40 |
| | Time spent active (sec) | Male | 646.9 ± 42.39 | 619.0 ± 63.45 | 623.3 ± 36.55 |
| | | Female | 607.8 ± 74.20 | 595.6 ± 64.95 | 624.5 ± 62.23 |
| | Distance travelled (mm) | Male | 21188 ± 3554 | 18871 ± 5954 | 19140 ± 3803 |
| | | Female | 17096 ± 4673 | 17996 ± 4994 | 20150 ± 6317 |
| | Distanced moved | Male | 5020 ± 624.9 | 4861 ± 632.6 | 4824 ± 298.2 |
| | without travelling (mm) | Female | 4737 ± 726.9 | 4679 ± 607.2 | 4974 ± 550.3 |

No significant behavioural differences were found between genotypes.

### Example 10

### Cholesterol and Pregnenolone Biosynthesis

Results

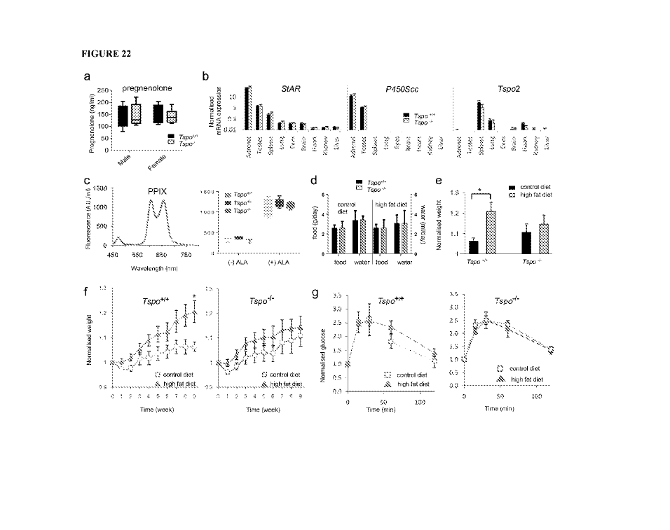

In both, male and female TSPO^{-/-} mice, all scrum pregnanolone concentrations, as measured by enzyme-linked immunosorbent assay (ELISA), were within the normal range seen in the colony control wild-type (TSPO^{+/+} male, 131±46.5 ng/ml; female: 150±36.5 ng/ml) and (TSPO^{-/-}: male, 145±44.8 ng/ml; female: 141±26.8 ng/ml) (FIG. 22a).

The enzymatic conversion of ³H-cholesterol to pregnenolone by P450Scc over a period of up to 2 hours as a read-out of mitochondrial cholesterol transport revealed no statistically significant differences in mean percentage conversion rates between all genotypes (TSPO^{+/+}: 14.35±4.6% (std); TSPO^{+/-}: 14.28±3.6% (std); TSPO^{-/-}: 15.6±4.0% (std); p>>0.05).

### Example 11

### P450Scc, StAR and TSPO2

Results

Likewise, quantitative RT-qPCR showed that the gene expression profiles for StAR, P455Scc and TSPO2 across the major organs known to express the TSPO were similar across genotypes, indicating that the global loss of TSPO had not led to any compensatory transcriptomic regulation of those genes thought to be most closely linked to TSPO as a regulator of steroidogenesis (FIG. 22b).

### Example 12

### Haematological Analysis and Biochemistry

Results

Analysis of blood, including cell sorting, demonstrated that the blood phenotype in both genders remained largely unaffected by the heterozygous or homozygous loss of the TSPO gene, the only exception being a statistically significant trend increase in natural killer (NK) cells in female TSPO^{-/-} mice compared to female TSPO^{+/+} (Table 4). Thus, the TSPO^{-/-} mice did not reveal the changes in cellular blood composition seen in zebra fish TSPO gene silencing experiments.

TABLE 4
Blood phenotyping

| analysis | phenotype | gender | TSPO^{+/+} | TSPO^{+/-} | TSPO^{-/-} |
|---|---|---|---|---|---|
| Haematology | Red blood cells (RBC) | Male | 9.220 ± 1.067 | 9.612 ± 1.117 | 9.560 ± 0.473 |
| | (×10⁶ cells/μL) | Female | 9.948 ± 0.591 | 10.36 ± 0.654 | 9.795 ± 0.756 |
| | White blood cell | Male | 7.428 ± 2.195 | 8.024 ± 3.957 | 8.440 ± 1.612 |
| | (WBC) (×10³ cells/μL) | Female | 10.21 ± 3.572 | 8.992 ± 3.205 | 8.635 ± 2.317 |
| | Platelets (×10³ | Male | 828.4 ± 479.6 | 1032 ± 302.8 | 1104 ± 143.4 |
| | cells/μL) | Female | 970.0 ± 184.7 | 846.0 ± 423.3 | 950.0 ± 94.84 |
| | Haematocrit (L/L) | Male | 0.454 ± 0.061 | 0.460 ± 0.052 | 0.464 ± 0.021 |
| | | Female | 0.486 ± 0.038 | 0.480 ± 0.027 | 0.472 ± 0.039 |
| | Reticulocytes (×10³ | Male | 332.6 ± 21.52 | 350.4 ± 26.28 | 341.6 ± 33.55 |
| | cells/μL) | Female | 377.8 ± 66.10 | 415.6 ± 83.99 | 380.8 ± 95.63 |
| | Haemoglobin (g/L) | Male | 102.0 ± 13.49 | 102.4 ± 9.63 | 102.4 ± 6.066 |
| | | Female | 109.2 ± 9.230 | 109.6 ± 6.986 | 105.5 ± 8.062 |
| | Mean corpuscular | Male | 98.20 ± 3.421 | 96.00 ± 2.121 | 97.20 ± 3.421 |
| | volume (fL) | Female | 97.60 ± 3.647 | 93.20 ± 1.095 | 96.00 ± 2.944 |
| Biochemistry | Na⁻ (mmol/L) | Male | 156.3 ± 2.645 | 157.3 ± 3.854 | 157.6 ± 1.989 |
| | | Female | 155.3 ± 2.490 | 153.9 ± 3.488 | 156.1 ± 1.792 |
| | K⁺ (mmol/L) | Male | 8.040 ± 0.744 | 7.675 ± 0.704 | 7.620 ± 0.349 |
| | | Female | 7.060 ± 0.770 | 6.850 ± 0.645 | 7.160 ± 0.550 |
| | Cl⁻ (mmol/L) | Male | 112.6 ± 0.894 | 112.0 ± 0.816 | 111.0 ± 1.871 |
| | | Female | 111.2 ± 0.837 | 112.0 ± 2.160 | 113.4 ± 2.881 |
| | Glucose (mmol/L) | Male | 9.550 ± 1.759 | 10.11 ± 1.417 | 10.13 ± 1.534 |
| | | Female | 10.06 ± 1.431 | 11.01 ± 2.393 | 11.55 ± 1.611 |
| | Cholesterol (mmol/L) | Male | 2.404 ± 0.706 | 2.707 ± 0.701 | 2.894 ± 0.458 |
| | | Female | 2.512 ± 0.622 | 2.967 ± 0.085 | 2.324 ± 0.671 |
| | Triglyceride (mmol/L) | Male | 1.000 ± 0.000 | 1.500 ± 0.577 | 1.600 ± 0.548 |
| | | Female | 1.200 ± 0.447 | 1.250 ± 0.500 | 1.138 ± 0.500 |
| | High density | Male | 1.800 ± 0.837 | 1.750 ± 0.500 | 2.000 ± 0.000 |
| | lipoprotein (mmol/L) | Female | 1.800 ± 0.447 | 2.000 ± 0.000 | 1.632 ± 0.507 |
| | Creatine kinase (U/L) | Male | 1507 ± 1254 | 2031 ± 1339 | 1067 ± 766.8 |
| | | Female | 1452 ± 1047 | 3576 ± 156.3 | 6417 ± 10647 |
| | Alanine | Male | 29.14 ± 9.492 | 52.98 ± 38.63 | 43.62 ± 6.454 |
| | aminotransferase (U/L) | Female | 36.22 ± 12.78 | 56.58 ± 13.78 | 81.98 ± 81.76 |
| | Bilirubin (mmol/μL) | Male | 4.100 ± 1.173 | 3.050 ± 0.819 | 4.200 ± 2.893 |
| | | Female | 3.180 ± 0.630 | 3.875 ± 0.640 | 4.100 ± 1.183 |
| Lymphocyte | Lymphocytes (%) | Male | 52.86 ± 4.794 | 51.80 ± 4.770 | 48.66 ± 6.494 |
| FACs | | Female | 54.86 ± 6.138 | 49.38 ± 5.053 | 49.96 ± 8.400 |
| | T-cells (%) | Male | 22.56 ± 5.854 | 19.90 ± 2.554 | 18.94 ± 2.214 |
| | | Female | 21.12 ± 4.395 | 18.66 ± 4.873 | 20.46 ± 5.793 |
| | CD4⁺ T-cells (%) | Male | 41.32 ± 5.295 | 40.56 ± 3.930 | 40.00 ± 3.845 |
| | | Female | 38.34 ± 4.702 | 36.40 ± 3.449 | 39.14 ± 3.745 |
| | Activated CD4⁺ T-cells | Male | 49.02 ± 9.648 | 47.14 ± 3.702 | 51.16 ± 3.233 |
| | (%) | Female | 48.30 ± 6.120 | 49.82 ± 6.994 | 48.90 ± 15.04 |
| | CD8⁻ T-cells (%) | Male | 33.68 ± 3.328 | 33.74 ± 3.702 | 34.46 ± 2.370 |
| | | Female | 34.10 ± 1.612 | 32.54 ± 5.192 | 32.04 ± 4.922 |
| | Activated CD8⁺ T-cells | Male | 36.74 ± 8.442 | 36.20 ± 7.563 | 36.16 ± 6.690 |
| | (%) | Female | 29.78 ± 5.325 | 30.68 ± 9.696 | 33.92 ± 6.479 |
| | B-cells (%) | Male | 56.52 ± 4.177 | 57.38 ± 3.433 | 60.56 ± 9.176 |
| | | Female | 60.40 ± 5.561 | 53.88 ± 6.548 | 56.28 ± 5.356 |
| | Mature B-cells (%) | Male | 5.980 ± 0.916 | 6.950 ± 1.022 | 6.580 ± 1.833 |
| | | Female | 6.548 ± 2.391 | 9.210 ± 2.962 | 7.686 ± 2.276 |
| | Immature B-cells (%) | Male | 88.58 ± 0.963 | 87.620 ± 1.190 | 87.00 ± 3.811 |
| | | Female | 86.58 ± 3.910 | 81.600 ± 6.005 | 83.88 ± 3.454 |
| | Double negative B- | Male | 4.610 ± 0.313 | 4.692 ± 0.504 | 5.668 ± 1.996 |
| | cells (%) | Female | 6.116 ± 1.396 | 8.316 ± 2.958 | 7.258 ± 1.092 |
| | Monocytes (%) | Male | 7.904 ± 0.990 | 8.080 ± 1.190 | 9.034 ± 2.280 |
| | | Female | 7.540 ± 1.273 | 9.420 ± 2.621 | 9.644 ± 2.240 |
| | IgE⁺ cells on | Male | 1.216 ± 0.906 | 2.142 ± 1.148 | 2.064 ± 1.583 |
| | lymphocytes (%) | Female | 1.338 ± 0.523 | 1.044 ± 0.677 | 0.994 ± 0.558 |
| | NK cells | Male | 3.990 ± 0.564 | 3.986 ± 0.734 | 3.542 ± 1.277 |
| | | Female | 3.580 ± 0.724 | 4.742 ± 1.359 | 7.102 ± 2.662* |
| | Activated NK cells (%) | Male | 48.20 ± 6.119 | 45.72 ± 6.433 | 42.38 ± 4.464 |
| | | Female | 28.34 ± 5.126 | 25.86 ± 3.325 | 25.66 ± 3.122 |
| Spleen | Lymphocytes (%) | Male | 58.86 ± 5.590 | 57.40 ± 4.094 | 62.12 ± 5.999 |
| FACs | | Female | 57.18 ± 4.124 | 56.00 ± 6.665 | 54.54 ± 4.787 |
| | T-cells (%) | Male | 27.94 ± 3.431 | 29.08 ± 3.163 | 27.94 ± 4.448 |
| | | Female | 27.80 ± 3.050 | 27.50 ± 6.131 | 25.06 ± 6.135 |
| | CD4⁺ T-cells (%) | Male | 53.50 ± 2.540 | 53.92 ± 3.330 | 52.70 ± 0.704 |
| | | Female | 53.40 ± 1.459 | 52.40 ± 4.743 | 53.90 ± 5.178 |
| | Activated CD4⁺ T-cells | Male | 45.06 ± 6.408 | 35.88 ± 5.585 | 35.78 ± 7.917 |
| | (%) | Female | 46.02 ± 6.423 | 42.58 ± 11.24 | 50.80 ± 4.904 |
| | CD8⁺ T-cells (%) | Male | 27.78 ± 1.293 | 27.58 ± 1.970 | 28.42 ± 0.729 |
| | | Female | 26.84 ± 0.844 | 26.72 ± 5.969 | 24.76 ± 4.438 |
| | Activated CD8⁺ T-cells | Male | 25.18 ± 5.134 | 23.52 ± 4.061 | 24.64 ± 7.823 |
| | (%) | Female | 22.10 ± 3.395 | 22.64 ± 8.740 | 27.08 ± 8.113 |
| | B-cells (%) | Male | 58.64 ± 2.071 | 57.96 ± 1.783 | 60.12 ± 3.805 |
| | | Female | 58.98 ± 3.650 | 59.40 ± 3.305 | 61.28 ± 6.465 |
| | Immature B-cells (%) | Male | 82.06 ± 1.009 | 80.58 ± 2.974 | 80.72 ± 1.254 |
| | | Female | 80.34 ± 2.648 | 77.74 ± 5.652 | 78.40 ± 4.128 |
| | Mature B-cells (%) | Male | 8.540 ± 0.152 | 9.940 ± 1.922 | 10.00 ± 1.578 |
| | | Female | 10.40 ± 2.723 | 13.12 ± 3.916 | 11.74 ± 3.440 |
| | Double negative B- | Male | 8.860 ± 0.924 | 8.860 ± 1.106 | 8.760 ± 1.324 |
| | cells (%) | Female | 8.860 ± 0.564 | 8.560 ± 1.826 | 9.360 ± 0.910 |
| | Monocytes (%) | Male | 5.080 ± 0.460 | 4.620 ± 0.356 | 5.140 ± 0.577 |
| | | Female | 5.880 ± 0.630 | 5.620 ± 0.522 | 5.540 ± 1.014 |
| | IgE⁺ cells on | Male | 1.000 ± 0.418 | 1.360 ± 0.513 | 0.760 ± 0.467 |
| | monocytes (%) | Female | 0.480 ± 0.455 | 0.520 ± 0.466 | 0.620 ± 0.130 |
| | NK cells (%) | Male | 2.460 ± 0.546 | 3.100 ± 1.298 | 2.100 ± 0.235 |
| | | Female | 2.140 ± 0.321 | 3.180 ± 1.262 | 3.200 ± 1.173 |
| | Activated NK cells (%) | Male | 49.44 ± 3.679 | 45.88 ± 4.492 | 46.02 ± 6.464 |
| | | Female | 39.54 ± 5.984 | 31.46 ± 1.576 | 35.40 ± 3.583 |

*The relative abundance of NK cells (%) in female TSPO^{-/-} mice were significantly greater than in female TSPO^{+/+} and in female TSPO^{+/-} mice (P < 0.05)

The concentrations of protoporphyrin IX (PPIX), thought to be an endogenous TSPO ligand, in the blood of TSPO^{+/+}, TSPO^{+/-} and TSPO^{-/-} mice were indistinguishable and increased similarly after treatment with 5-aminolevulinic acid (ALA), the first intermediate in haem biosynthesis and a precursor of PPIX (FIG. 22c), thus indicating normal haem metabolism.

### Example 13

### Mitochondrial Metabolism and Response to High Fat Diet

Results

While there was no difference in weight gain between TSPO^{+/+} and TSPO^{-/-} animals on a standard diet, TSPO^{-/-} animals on a high fat diet had a significantly reduced relative weight gain, unlike TSPO^{+/+} animals, despite the same food and water intake (FIG. 22d to 22f and Table 5). No significant change in glucose tolerance due to the high fat diet was observed in TSPO^{-/-}animals while TSPO^{+/+} animals showed the expected trend towards glucose intolerance (FIG. 22g).

TABLE 5
Diet composition

| | High fat diet | | Control | | Premium breadder | |
|---|---|---|---|---|---|---|
| | (SF03-002) - | | diet(AIN93M) - | | diet - Gordon's | |
| | Specialty Feeds | | Specialty Feeds | | Specialty Stockfeeds | |
| Calculated Nutritional Parameters | | | | | | |
| Protein | 19.40% | | 13.60% | | 23% | |
| Total Fat | 36.00% | | 4.00% | | 6% | |
| Total digestible carbohydrate as | No data | | 64.80% | | | |
| defined by FSANZ Standard 1.2.8 | | | | | | |
| Crude Fibre | 4.70% | | 4.70% | | 5% | |
| AD Fibre | 4.70% | | 4.70% | | | |
| Digestible Energy | 22.8 | MJ/kg | 15.1 | MJ/kg | | |
| Metabolisable Energy | | | | | 13 | MJ/kg |
| % Total calculated digestible | 59.00% | | 9.00% | | | |
| energy from lipids | | | | | | |
| % Total calculated digestible | 15.00% | | 15.00% | | | |
| energy from protein | | | | | | |
| Ingredients | | | | | | |
| Casein (Acid) | 200 | g/kg | 140 | g/kg | | |
| Sucrose | 346 | g/kg | 100 | g/Kg | | |
| Canola Oil | 60 | g/kg | 40 | g/kg | | |
| Cocoa Butter | 240 | g/kg | | | | |
| Hydrogenated Vegetable Oil | 60 | g/kg | | | | |
| (Copha) | | | | | | |
| Cellulose | 50 | g/kg | 50 | g/kg | | |
| DL Methionine | 3.0 | g/kg | 1.8 | g/kg | | |
| Wheat Starch | | | 472 | g/kg | | |
| Dextrinised Starch | | | 155 | g/kg | | |
| Calcium Carbonate | 13.1 | g/kg | 13.1 | g/kg | | |
| Sodium Chloride | 2.6 | g/kg | 2.6 | g/kg | | |
| AIN93 Trace Minerals | 1.4 | g/kg | 1.4 | g/kg | | |
| Potassium Citrate | 2.5 | g/kg | 1.0 | g/kg | | |
| Potassium Dihydrogen Phosphate | 6.9 | g/kg | 8.8 | g/kg | | |
| Potassium Sulphate | 1.6 | g/kg | 1.6 | g/kg | | |
| Choline Chloride (75%) | 2.5 | g/kg | 2.5 | g/kg | | |
| AIN93 Vitamins | 10 | g/kg | 10 | g/kg | | |
| Antioxidant (Oxicap E2) | 0.04 | g/kg | | | | |
| Calculated Amino Acids | | | | | | |
| Arginine | | | | | 1.21% | |
| Valine | 1.30% | | 0.90% | | 1.02% | |
| Leucine | 1.80% | | 1.30% | | 1.52% | |
| Isoleucine | 0.90% | | 0.60% | | 0.80% | |
| Threonine | 0.80% | | 0.60% | | 0.84% | |
| Methionine | 0.80% | | 0.60% | | 0.59% | |
| Cystine | 0.06% | | 0.05% | | | |
| Lysine | 1.50% | | 1.00% | | 0.98% | |
| Phenylalanine | 1.00% | | 0.70% | | 1.64% | |
| Tyrosine | 1.00% | | 0.70% | | 0.37% | |
| Tryptophan | 0.30% | | 0.20% | | | |
| Histidine | 0.60% | | 0.42% | | 0.50% | |
| Calculated Total Minerals | | | | | | |
| Calcium | 0.46% | | 0.47% | | 1.01% | |
| Phosphorous | 0.32% | | 0.35% | | 0.77% | |
| Magnesium | 0.09% | | 0.09% | | 0.18% | |
| Sodium | 0.12% | | 0.15% | | 0.30% | |
| Chloride | 0.16% | | 0.16% | | | |
| Potassium | 0.40% | | 0.40% | | 0.54% | |
| Sulphur | 0.20% | | 0.17% | | | |
| Iron | 72 | mg/kg | 75 | mg/kg | 97.0 | mg/kg |
| Copper | 7.0 | mg/kg | 6.9 | mg/kg | 10.6 | mg/kg |
| Iodine | 0.2 | mg/kg | 0.2 | mg/kg | 1.15 | mg/kg |
| Manganese | 18 | mg/kg | 19.5 | mg/kg | 87.4 | mg/kg |
| Cobalt | No data | | No data | | | |
| Zinc | 51 | mg/kg | 47 | mg/kg | 48.1 | mg/kg |
| Molybdenum | 0.15 | mg/kg | 0.15 | mg/kg | | |
| Selenium | 0.3 | mg/kg | 0.3 | mg/kg | 0.1 | mg/kg |
| Cadmium | No data | | No data | | | |
| Chromium | 1.0 | mg/kg | 1.0 | mg/kg | | |
| Fluoride | 1.0 | mg/kg | 1.0 | mg/kg | | |
| Lithium | 0.1 | mg/kg | 0.1 | mg/kg | | |
| Boron | 2.1 | mg/kg | 3.1 | mg/kg | | |
| Nickel | 0.5 | mg/kg | 0.5 | mg/kg | | |
| Vanadium | 0.1 | mg/kg | 0.1 | mg/kg | | |
| Calculated Total Vitamins | | | | | | |
| Vitamin A (Retinol) | 4 000 | IU/kg | 4 000 | IU/kg | 5666 | IU/kg |
| Vitamin D (Cholecalciferol) | 1 000 | IU/kg | 1 000 | IU/kg | 200 | IU/kg |
| Vitamin E (a Tocopherol acetate) | 86 | mg/kg | 75 | mg/kg | 50 | mg/kg |
| Vitamin K (Menadione) | 1 | mg/kg | 1 | mg/kg | 5 | mg/kg |
| Vitamin C (Ascorbic acid) | | | | | 150 | mg/kg |
| Vitamin B1 (Thiamine) | 6.1 | mg/kg | 6.1 | mg/kg | 4 | mg/kg |
| Vitamin B2 (Riboflavin) | 6.3 | mg/kg | 6.3 | mg/kg | 5 | mg/kg |
| Niacin (Nicotinic acid) | 30 | mg/kg | 30 | mg/kg | 10 | mg/kg |
| Vitamin B6 (Pryridoxine) | 7 | mg/kg | 7 | mg/kg | 6 | mg/kg |
| Pantothenic Acid | 16.5 | mg/kg | 16.5 | mg/kg | 12 | mg/kg |
| Biotin | 200 | μg/kg | 200 | μg/kg | 0.06 | mg/kg |
| Folic Acid | 2 | mg/kg | 2 | mg/kg | 10 | mg/kg |
| Inositol | | | | | | |
| Vitamin B12 (Cyancobalamin) | 103 | μg/kg | 103 | μg/kg | 5 | μg/kg |

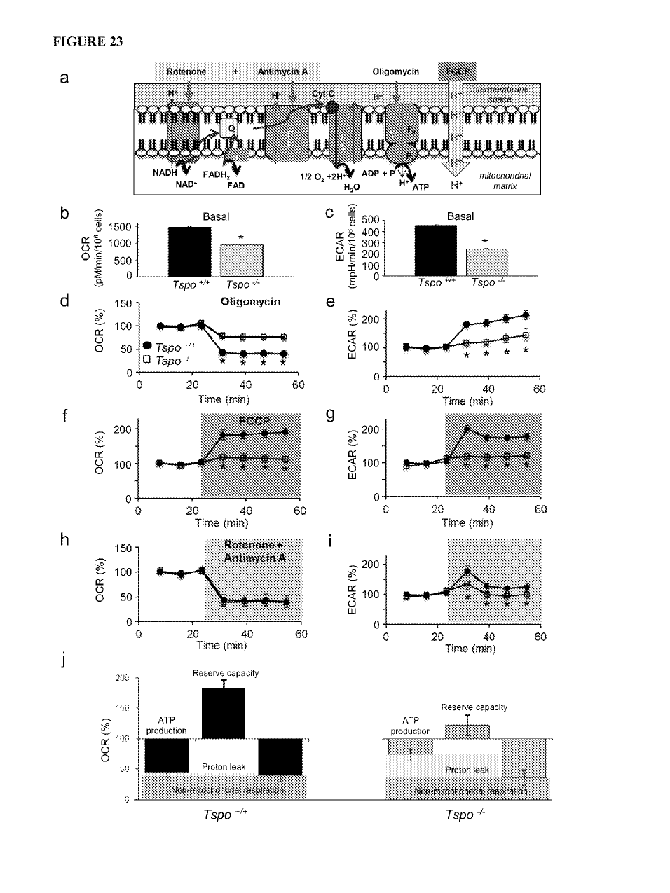

Comparative analysis of mitochondrial respiration and ATP production in primary cultures of microglia, cell rich in mitochondria, from TSPO^{+/+} and TSPO^{-/-} mice using oxygen and proton sensitive fluorophores, as well as selective inhibitor or uncoupling agents of the electron transport chain (FIG. 23a), revealed that TSPO^{-/-} mitochondria compared to wild-type have:
1) a significantly lower basal mitochondrial oxygen consumption rate (OCR) (FIG. 23b) and
2) reduced ATP production after inhibition of complex V with oligomycin (FIG. 23d);
3) a significantly reduced maximal respiration (reserve capacity) after uncoupling, i.e. reduction of the proton gradient with FCCP (FIG. 23f); and
4) and a significantly increased proton leak (FIG. 23j), as calculated by subtraction of the oligomycin-inhibited OCR (shown in FIG. 23d) from the OCR under combined rotenone and antimycin A inhibition of complexes I and III (FIG. 23h).

The reduced ATP production by TSPO^{-/-} mitochondria was not associated with a compensatory increase in glycolysis as measured by extracellular acidification rates (ECAR) (FIGS. 23c, 23c, 23g and 23i), indicating that the loss in ATP production due to a greater proton leak was not compensated through non-mitochondrial pathways.

### Examples 8 to 13

### Discussion

Orthologues of TSPO are found widely among eubacteria, archaea bacteria and eukaryotes (Gavish, M. et al. Enigma of the peripheral benzodiazepine receptor. Pharmacol Rev 51, 629-650 (1999); Papadopoulos, V. et al. Translocator protein (8 kDa): new nomenclature for the peripheral-type benzodiazepine receptor based on its structure and molecular function. Trends Pharmacol Sci 27, 402-409, doi:S0165-6147(06)00153-2 [pii]10.1016/j.tips.2006.06.005 (2006)). Most members of the TSPO protein family contain a specific binding site for the isoquinoline carboxamide PK11195 which has historically been used to pharmacologically define the TSPO and distinguish it from other benzodiazepine binding receptors, such as the GABA_{A} receptor protein complex (Gavish, M. et al. Enigma of the peripheral benzodiazepine receptor. Pharmacol Rev 51, 629-650 (1999); Le Fur, G. et al. Peripheral benzodiazepine binding sites: effect of PK11195, I-(2-chlorophenyl)-N-methyl-(1-methylpropyl)-3 isoquinolinecarboxamide. II. In vivo studies. Life Sci 32, 1849-1856 (1983)). An evolutionarily younger C-terminal cholesterol recognition amino acid consensus (CRAC) domain is primarily found in the animal phylum. A paralogue of TSPO, TSPO2, exists in birds and mammals as the result of a gene duplication, which has retained the CRAC domain but lost the isoquinoline binding site (Fan, J., Rone, M. B. & Papadopoulos, V. Translocator protein 2 is involved in cholesterol redistribution during crythropoiesis. J Biol Chem 284, 30484-30497, doi: 10.1074/jbc.M 109.029876 (2009)).

In light of the apparent evolutionary pressure to conserve core functions of the gene, and the protein's reported vital role in steroidogenesis, it was surprising to find that a global TSPO knock-out animal according to the present invention not only survived but displayed an overtly “normal” phenotype

Notwithstanding, and while the functional impact on the TSPO knock-out has been shown to be discreet the experimental data outlined in the examples above indicate an important role for TSPO and TSPO-mediated signalling in the cellular and systemic energy household. Specifically, the data presented here shows that prolonged increase in energy intake through high fat diet leads to a significantly reduced (and less than expected) weight gain in the TSPO knock-out animals when compared to wild-type animals thereby providing a role for TSPO and TSPO mediated signalling in the protection against obesity resulting from a high fat diet. Similarly, the data indicates that TSPO plays a role in energy storage which appears to be less efficient in the absence of TSPO. Accordingly, TSPO most likely plays a role in the activation of mitochondrial oxidative pathways and the cellular measurements suggest that the less efficient energy storage observed in TSPO knock-out animals is due to an increased mitochondrial proton leak.

The results presented in accordance with embodiments of the present invention may help to elucidate how TSPO-mediated changes in the cellular and systemic energy household (measured as changes in ATP production) could explain indirect regulatory effects on steroid biogenesis, which is energy-dependent. However, the surprising observations made possible by the generation of the transgenic animal according to the present invention now allows for the re-evaluation and potential validation or, alternatively, dismissal of previously generated data relating to TSPO and its in vivo function and relevance. Notably, this includes studies using compounds with purported selectivity and specificity for TSPO in the methods and uses according to the present invention.

Thus, while there has been described what are believed to be the preferred embodiments of the invention, those skilled in the art will recognize that other and further modifications may be made thereto without departing from the spirit of the invention, and it is intended to claim all such changes and modifications as falling within the scope of the invention.

1: A transgenic, non-human animal comprising genetically engineered cells with at least one copy of a non-functional, endogenous translocator protein (TSPO) gene.

2: The transgenic, non-human animal of claim 1, wherein said cells do not comprise a functional TSPO gene.

3: The transgenic, non-human animal of claim 1, wherein said non-functional, TSPO gene contains at least one mutation selected from the group consisting of a deletion, an insertion, a frame-shift mutation, a re-arrangement and a substitution.

4: The transgenic, non-human animal of claim 1, wherein the said mutation is constitutive.

5. (canceled)

6: The transgenic, non-human animal of claim 4, wherein the said mutation comprises a deletion of all or part of exon 1, 2, 3 and/or 4 within said TSPO gene, or wherein the said mutation comprises a deletion of all or part of exon 2 and/or 3 within said TSPO gene.

7-8. (canceled)

9: The transgenic, non-human animal of claim 1, wherein the animal is a mouse.

10: Progeny of the transgenic, non-human animal of claim 1.

11: The progeny generated from cross-breeding the mouse of claim 9 with a pKZ1 mouse, a Brca2 homozygous mouse, Tg(CAT)(+/+) mouse, an A-T mutated heterozygous/homozygous mouse, a Csbm/m mouse, an insulin-like growth factor 1heterozygous and homozygous mouse, a P53 heterozygous and homozygous mouse, a radiation sensitive and resistant transgenic mouse, a Schizophrenia DISC1 knock-out mouse, a Schizophrenia neuregulin 1 knock-out mouse, a genetic engineering model tumour mouse, a neuroinflammation model mouse, an Alzheimer's Disease model mouse, a Parkinson's disease model mouse or a mouse with targeted deletion of the type 2 deiodinase gene (D2KO) that is insulin resistant and susceptible to diet induced obesity.

12-16. (canceled)

17: A method for using the transgenic, non-human animal of claim 1, or a progeny thereof, comprising use of the animal or progeny:
(a) (i) as a negative control for detecting a TSPO gene product in a biological sample;
(ii) for the diagnosis of a TSPO-related disease or disorder in a subject;
(iii) for identifying a compound for use in the treatment of a TSPO-related disease or disorder in a subject; or
(iv) for screening the binding specificity or selectivity of a candidate compound for use in the treatment of a TSPO-related disease or disorder in a subject;
(b) the method of (a), wherein the TSPO-related disease or disorder is selected from the group consisting of cancer, neuroinflammation, Alzheimer Disease, Parkinson's Disease, Epilepsy, brain injury, Ischemia-reperfusion injury, behaviour or neurological or psychiatric disorders including acute and chronic stress, anxiety disorders, mode disorders, and Schizophrenia, peripheral neuropathy, Multiple Sclerosis, neuropathic pain, obesity, diabetes and cachexia.

18: A cell, tissue or immortalised cell line derived from the transgenic, non-human animal of claim 1.

19-24. (canceled)

25: A method for identifying a compound for use in the treatment of a TSPO-related disease or disorder, the method comprising:
(A)
(a) (i) administering a candidate compound to the transgenic, non-human animal of claim 1 or, (ii) exposing a cell, tissue or immortalised cell line derived from the transgenic, non-human animal, to a candidate compound, and
(b) assessing the effects of the candidate compound on: (i) the phenotype of said cell, tissue or immortalised cell line; or (ii) the expression levels of TSPO-associated gene products, or any TSPO gene products that may be present in the non-human animal, cell, tissue or immortalised cell line,
(B) the method of (A), wherein the TSPO-related disease or disorder is selected from the group consisting of cancer, neuroinflammation, Alzheimer Disease, Parkinson's Disease, Epilepsy, brain injury, Ischemia-reperfusion injury, behaviour or neurological or psychiatric disorders including acute and chronic stress, anxiety disorders, mode disorders, and Schizophrenia, peripheral neuropathy, Multiple Sclerosis, neuropathic pain, obesity, diabetes and cachexia.

26: (canceled)

27: A method for screening the binding specificity or selectivity of a candidate compound for use in the treatment of a TSPO-related disease or disorder in a subject, the method comprising:
(a) (i) administering a candidate compound to a transgenic, non-human animal of claim 1 and to a wild-type non-human animal of the same species, and
(ii) comparing the binding specificity or selectivity of the candidate compound to TSPO-associated gene products, or any TSPO gene products that may be present in the wild-type cell, wild-type tissue or wild-type immortalised cell line, with the binding specificity or selectivity of the candidate compound to TSPO-associated gene products, or any TSPO gene products that may be present in the transgenic, non-human animal,
(b) the method of (a), wherein the TSPO-related disease or disorder is selected from the group consisting of cancer, neuroinflammation, Alzheimer Disease, Parkinson's Disease, Epilepsy, brain injury, Ischemia-reperfusion injury, behaviour or neurological or psychiatric disorders including acute and chronic stress, anxiety disorders, mode disorders, and Schizophrenia, peripheral neuropathy, Multiple Sclerosis, neuropathic pain, obesity, diabetes and cachexia.

28-32. (canceled)

33: A method of: (a) inhibiting TSPO function; (b) altering the systemic and/or cellular energy household; (c) altering a mitochondrial oxidative pathway (d) regulating mitochondrial ATP production; (e) regulating TSPO-mediated signalling; (f) regulating TSPO-mediated energy storage; or (g) investigating inflammatory responses associated with neuronal injury, comprising the administration of a compound of general formula (I) 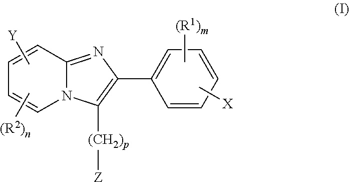
to inhibit TSPO function, wherein
X is absent, iodine or an isotope thereof;
Y is selected from F, Cl, Br, I, OH, SH, NH₂, CN and COOH;
Z is selected from N(R³)C(O)R⁴ and C(O)NR³R⁴;
R¹ and R² are independently selected from (C₁-C₆)alkyl, (C₁-C₆)alkoxy, (C₂-C₆)alkenyl, (C₂-C₆)alkynyl, (C₃-C₆)cycloalkyl, (C₆-C₁₂)aryl, (C₆-C₁₂)aryloxy, (C₆-C₁₂)aryl(C₁-C₆)alkyl, heteroaryl, heteroaryl(C₁-C₆)alkyl, heterocyclic, (C₂-C₆)alkanoyl and (C₂-C₇)acyl, each of which may be unsubstituted or substituted with from 1 to 3 substituents selected from the group consisting of halogen or an isotope thereof, OH, (C₁-C₄)alkoxy, SH, NH₂, (C₁-C₄)alkylamino, di((C₁-C₄)alkyl)amino, carboxy, (C₁-C₄)alkoxycarbonyl, (C₂-C₄)alkanoyl, oxo, amido, CN, CNS, SCN, CNO, OCN and NHOH;
R³ and R⁴ are each independently hydrogen or a group selected from (C₁-C₄)alkyl, (C₂-C₄)alkenyl, (C₂-C₄)alkynyl, (C₃-C₆)cycloalkyl, (C₆-C₁₂)aryl, (C₆-C₁₂)aryl(C₁-C₄)alkyl, heteroaryl, heteroaryl(C₁-C₄)alkyl, heterocyclic, (C₁-C₄)alkoxycarbonyl and (C₂-C₅)acyl, each of which may be unsubstituted or substituted with from 1 to 3 substituents selected from the group consisting of halogen, OH, (C₁-C₄)alkoxy, SH, NH₂, (C₁-C₄)alkylamino, di((C₁-C₄)alkyl)amino, carboxy, (C₁-C₄)alkoxycarbonyl, (C₁-C₄)alkanoyl, oxo. amido, CN, CNS, SCN, CNO, OCN and NHOH,
or R³ and R⁴ together are (C₂-C₇)alkylidene which may be optionally substituted with from 1 to 3 substituents selected from the group consisting of halogen, OH, (C₁-C₄)alkoxy, SH, NH₂, (C₁-C₄)alkylamino, di((C₁-C₄)alkyl)amino, carboxy, (C₁-C₄)alkoxycarbonyl, (C₁-C₄)alkanoyl, oxo, amido, CN, CNS, SCN, CNO, OCN and NHOH;
m and n are independently 0, 1 or 2; and
p is 1;
wherein at each occurrence
(i) alkenyl has the meaning of ethylenically mono- or di-unsaturated alkyl or ethylenically mono- or di-unsaturated cycloalkyl;
(ii) cycloalkyl has the meaning of cyclic alkyl, or alkyl substituted cyclic alkyl;
(iii) heteroaryl has the meaning of single, polynuclear, conjugated and fused residues of aromatic heterocyclic ring systems.

34-39. (canceled)

40: The method of claim 33, wherein said administration provides protection against obesity and/or high fat diet-induced weight gain.

41-48. (canceled)

49: The method of claim 40, wherein the compound comprises [¹²⁵I]CLINDE or [¹⁸F]PBR111.

50: (canceled)

51: A method for using a cell, tissue or immortalised cell line of claim 18, comprising use of the cell, tissue or immortalised cell line:
(a) as a negative control for detecting a TSPO gene product in a biological sample;
(b) for the diagnosis of a TSPO-related disease or disorder in a subject;
(c) for identifying a compound for use in the treatment of a TSPO-related disease or disorder in a subject; or
(d) for screening the binding specificity or selectivity of a candidate compound for use in the treatment of a TSPO-related disease or disorder in a subject.

52: The method of claim 51, wherein the TSPO-related disease or disorder is selected from the group consisting of cancer, neuroinflammation, Alzheimer Disease, Parkinson's Disease, Epilepsy, brain injury, Ischemia-reperfusion injury, behaviour or neurological or psychiatric disorders including acute and chronic stress, anxiety disorders, mode disorders, and Schizophrenia, peripheral neuropathy, Multiple Sclerosis, neuropathic pain, obesity, diabetes and cachexia.
